SEQUENCE LISTING (52 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, Artificial Sequence
  source: /note=“Description of Artificial Sequence Synthetic polypeptide”
DIQVTQSPSSLSASVGDRVTITCITSTDIDDDMNWYQQKPGKVPKLLISGGNTLRPGVPSRFSGSGSGTDFTLTISSLQPEDVATYYCLQSDSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 223 aa, Artificial Sequence
  source: /note=“Description of Artificial Sequence Synthetic polypeptide”
EVQLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

(19) United States
(12) Patent Application Publication
(10) Pub. No.: US 2023/0172930 A1
(43) Pub. Date: Jun. 8, 2023

(54) USE OF COMPLEMENT FACTOR D INHIBITORS ALONE OR IN COMBINATION WITH ANTI-C5 ANTIBODIES FOR TREATMENT OF PAROXYSMAL NOCTURNAL HEMOGLOBINURIA

(71) Applicant: Alexion Pharmaceuticals, Inc., Boston, MA (US)

(72) Inventors: Mingjun Huang, New Haven, CT (US); James Hui, Apex, NC (US); Steven Podos, New Haven, CT (US); Dharaben Patel, Orange, CT (US); Michael Geffner, Morristown, NJ (US)

(73) Assignee: Alexion Pharmaceuticals, Inc., Boston, MA (US)

(21) Appl. No.: 17/924,518

(22) PCT Filed: May 11, 2021
(86) PCT No.: PCT/US2021/031832

§ 371 Date: Nov. 10, 2022

Related U.S. Application Data
(60) Provisional application No. 63/023,415, filed on May 12, 2020.
(60) Provisional application No. 63/044,431, filed on Jun. 26, 2020.

(51) Int. Cl. A61K 31/506; C07K 16/18; A61P 7/00

(52) U.S. Cl. CPC A61K 31/506; C07K 16/18; A61P 7/00

(57) ABSTRACT

Provided are methods for treating paroxysmal nocturnal hemoglobinuria in a subject who previously exhibited an inadequate response to an anti-C5 antibody therapy, by administering to the subject a therapeutically effective amount of an inhibitor of the alternative pathway of complement (e.g., one which inhibits a target upstream to complement 5 (C5), such as Factor D or complement 3 (C3)). Also provided herein are methods for treating PNH in a human subject, comprising administering to the subject a complement factor D inhibitor alone or in combination with an anti-C5 antibody, or antigen binding fragment thereof. In some embodiments, the patient previously exhibited an inadequate response to an anti-C5 antibody therapy.

### CROSS REFERENCE TO RELATED APPLICATIONS

This application claims priority to, and the benefit of, U.S. Provisional Application No. 63/023,415 (filed on May 12, 2020) and U.S. Provisional Application No. 63/044,431 (filed on Jun. 26, 2020), the entire contents which are incorporated herein by reference.

### SEQUENCE LISTING

The instant application contains a Sequence Listing which has been submitted electronically in ASCII format and is hereby incorporated by reference in its entirety. Said ASCII copy, created on May 11, 2021, is named 0618_WO_SL.txt and is 62,966 bytes in size.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

While a properly functioning complement system provides a robust defense against infecting microbes, inappropriate regulation or activation of the complement pathways has been implicated in the pathogenesis of a variety of disorders, including paroxysmal nocturnal hemoglobinuria (PNH). PNH is a condition in which uncontrolled complement activity leads to systemic complications, principally through intravascular hemolysis and platelet activation (see Socié G, et al., French Society of Haematology. Lancet. 1996; 348(9027):573-577 and Brodsky, R., Blood. 2014; 124(18):2804-2811). Persistent intravascular hemolysis may be triggered by various stressors, such as infection or physical exertion, and this leads to smooth muscle contraction (free hemoglobin), chronic anemia, and an increased risk of severe thromboembolism. Thromboembolism is the most common cause of mortality in patients with PNH, and pulmonary hypertension and end-organ damage of vital organs, such as the liver, kidneys, brain, and intestines, are sequelae of such events (Hillmen, P., et al, Am. J. Hematol. 2010; 85(8):553-559). Due to these adverse pathologic processes, patients with PNH have a decreased quality of life (QoL), which may include debilitating fatigue, chronic pain, poor physical function, shortness of breath, abdominal pain, erectile dysfunction, a need for anticoagulation, blood transfusions and in some instances, need for dialysis (Weitz, I C., et al., Thromb Res. 2012; 130(3):361-368).

Patients with PNH are at risk of substantial morbidity and mortality. Accordingly, it is an object of the present disclosure to provide improved methods for treating patients with PNH.

### SUMMARY

The instant disclosure relates, in part, to the discovery that PNH patients who respond inadequately or fail to respond to anti-C5 antibody therapy benefit from treatment with an alternate inhibitor of complement, such as, Factor D (FD) inhibitor or C3 inhibitor. Specifically, transfusion dependent PNH patients on eculizumab receiving an oral FD inhibitor (danicopan) in addition to their usual regimen of eculizumab exhibited improved clinical outcomes, as evidenced by increase in haemoglobin (Hgb) levels, improved Functional Assessment of Chronic Illness Therapy (FACIT)—Fatigue score, reduction in transfusion needs, and improvement in other PNH parameters. The data show that blocking FD with FD inhibitors, such as danicopan, provides additional benefit in PNH patients who are on mainstay therapy with C5 inhibitors such as, e.g., anti-C5 antibody therapy with eculizumab (SOLIRIS®). This added benefit is likely due to the prevention of C3-mediated extracellular hemolysis (EVH), in addition to control of intravascular hemolysis (IVH).

Provided herein are methods for treating paroxysmal nocturnal hemoglobinuria (PNH) in a subject who previously exhibited an inadequate response to C5 inhibitors, e.g., an anti-C5 antibody therapy, by administering to the subject a therapeutically effective amount of an inhibitor of an alternate component of the alternative pathway (AP). In some embodiments, the inhibitor of the alternate component of the AP is one which inhibits a target upstream to complement 5 (C5), such as Factor D or complement 3 (C3). In some embodiments, the PNH subject has extravascular hemolysis (EVH).

In some embodiments, the treatment results in a reduction in one or more of the following in the subject: (a) persistent extravascular hemolysis (EVH); (b) anemia; and/or (c) transfusion dependence. In some embodiments, the treatment results in an improvement in FACIT Fatigue Scale Score. In some embodiments, the control of MAC-mediated intravascular hemolysis in the inadequately responding PNH subject is maintained or improved following treatment.

In some embodiments, the inadequate response an anti-C5 antibody therapy is related to a pharmacokinetic (PK) aspect, for example, (a) ineffective inhibition of C5 cleavage in the subject; (b) low dose and/or low subject plasma levels of the anti-C5 antibody; (c) enhanced clearance of the anti-C5 antibody in the subject; and/or (d) anti-C5 antibody intolerance in the subject resulting in lowered anti-C5 antibody dosing, preferably wherein anti-C5 antibody intolerance comprises fatigue and post-infusion pain. In some embodiments, the inadequate response an anti-C5 antibody therapy is related to a pharmacodynamic (PD) aspect, for example, (a) CR1 polymorphism; (b) extra-vascular hemolysis (EVH), e.g., via opsonization of blood cells surviving intra-vascular hemolysis (IVH); and/or (c) impaired effect of anti-C5 antibody activity by C3 fragments. In some embodiments, the inadequate response an anti-C5 antibody therapy is related to one or more PK and PD aspects.

Also provided herein are methods for treating PNH in a human patient, comprising administering to the patient a complement factor D (CFD) inhibitor alone or in combination with an anti-C5 antibody, or antigen binding fragment thereof. In some embodiments, the CFD inhibitor and/or anti-C5 antibody, or antigen binding fragment thereof, are administered (or are for administration) according to a particular clinical dosage regimen (e.g., at a particular dose amount and according to a specific dosing schedule). In some embodiments, the PNH subject has extravascular hemolysis (EVH). In some embodiments, the disclosure relates to method of treating clinically-evident extravascular hemolysis (EVH) in a patient (e.g., human patient) suffering from paroxysmal nocturnal hemoglobinuria (PNH). Particularly, embodiments of the disclosure relate to treating EVH in a PNH patient who has previously been treated with a C5 inhibitor, such as anti-C5 antibody (e.g., therapy with eculizumab or ravulizumab), comprising administering to the patient a therapeutically effective amount of a modulator (e.g., inhibitor) of an alternate component of the alternative pathway (AP) of complement. In some embodiments, the modulator of the alternate component of the AP of complement comprises inhibitor of a target upstream to complement 5 (C5), such as inhibitor of Factor D (FD) or complement 3 (C3); especially, inhibitor of Factor D.

In some embodiments of the therapeutic methods of the foregoing or following, the disclosure relates to method(s) for treating EVH in a PNH patient who has previously been treated with an C5 inhibitor, e.g., anti-C5 antibody therapy, comprising administering to the subject a therapeutically effective amount of a Factor D inhibitor, e.g., danicopan. In some embodiments, the therapeutically effective amount of danicopan is dosed at 600 mg per day.

In some embodiments of the therapeutic methods of the foregoing or following, the clinically evident EVH comprises (a) anemia (e.g., Hgb≤9.5 g/dL) with absolute reticulocyte count ≥120×10⁹/L; and/or (b) at least 1 packed RBC or whole blood transfusion within 6 months prior to the therapy with the inhibitor of the alternate component of the AP of complement, e.g., prior to therapy with a FD inhibitor such as danicopan.

In some embodiments of the therapeutic methods of the foregoing or following, the administration of the alternative component of the AP of complement, e.g., Factor D inhibitor such as danicopan, results in transfusion avoidance (TA) in the PNH patient with clinically-evident EVH.

In some embodiments of the therapeutic methods of the foregoing or following, the administration of alternate component of the AP complement pathway, e.g., Factor D inhibitor such as danicopan, results in the PNH patients with clinically-evident EVH becoming free of pRBC transfusion requirement, e.g., a requirement that the PNH patient undergo pRBC transfusion when the patient has a (1) hemoglobin value of less than 6 g/dL regardless of presence of clinical signs or symptoms of PNH; or (2) hemoglobin value of less than 9 g/dL with PNH signs or symptoms of sufficient severity to warrant a transfusion.

In some embodiments of the therapeutic methods of the foregoing or following, the PNH patient with clinically-evident EVH is treated with an anti-C5 antibody together with the therapeutically effective amount of the inhibitor of an alternate component of the AP complement, e.g., eculizumab or ravulizumab (as anti-C5 antibody) together with an FD inhibitor such as danicopan. In some embodiments, the patient is treated with FD inhibitor alone, e.g., with danicopan alone.

In some embodiments of the therapeutic methods of the foregoing or following, the PNH patient with clinically-evident EVH is treated with an anti-C5 antibody, e.g., eculizumab (SOLIRIS®) or ravulizumab (ULTOMIRIS®), per standard dosage and/or dosing schedule for the anti-C5 antibody in PNH therapy, prior to treatment with the inhibitor of the alternate component of AP of complement, e.g., treatment with Factor D inhibitor such as danicopan, and subsequently thereafter treated with the same anti-C5 antibody.

In some embodiments, the disclosure relates to use of an effective amount of a modulator (e.g., inhibitor) of an alternate component of the alternative pathway (AP) of complement for treating clinically-evident EVH in a patient, e.g., human patient, suffering from PNH. Particularly, embodiments of the disclosure relate to use of an effective amount of an inhibitor of a target upstream to C5 (such as inhibitor of FD or C3; especially, inhibitor of FD), in treating EVH in a PNH patient who has previously been treated with a C5 inhibitor such as anti-C5 antibody (e.g., therapy with eculizumab or ravulizumab). In particular embodiments, the disclosure relates to use of an effective amount of danicopan, e.g., an oral dose of 600 mg daily, in treating a human PNH patient with clinically-evident EVH, which patient has been previously treated with eculizumab or ravulizumab.

In one embodiment, a method for treating PNH in a subject is provided, the method comprising: administering to the subject a therapeutically effective amount of a complement factor D (CFD) inhibitor in combination with a therapeutically effective amount of an anti-C5 antibody, or antigen binding fragment thereof, wherein the subject exhibits one or more of the following clinical improvements 12 and/or 24 weeks post-treatment with the CFD inhibitor:

(a) hemoglobin increase of 2.0 g/dL or greater compared to the subject's baseline hemoglobin level;

(b) transfusion independence; and/or

(c) FACIT Fatigue Scale Score increase of 10 points or greater (e.g., 10, 11, 12) compared to the subject's baseline FACIT Fatigue Scale Score.

In another embodiment, a method for treating PNH in a subject who previously exhibited an inadequate response to an anti-C5 antibody therapy is provided, the method comprising:

administering to the subject a therapeutically effective amount of a complement factor D (CFD) inhibitor,

wherein the inadequate response by the subject was transfusion dependence and/or anemia; and

wherein the subject exhibits one or more of the following clinical improvements 12 and/or 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater (e.g., 10, 11, 12) compared to the subject's baseline FACIT Fatigue Scale Score.

In another embodiment, a method for treating PNH in a subject who previously exhibited an inadequate response to an anti-C5 antibody therapy is provided, the method comprising:

administering to the subject a therapeutically effective amount of a complement factor D (CFD) inhibitor in combination with a therapeutically effective amount of an anti-C5 antibody, or antigen binding fragment thereof,

wherein the inadequate response by the subject was transfusion dependence and/or anemia; and

wherein the subject exhibits one or more of the following clinical improvements 12 and/or 24 weeks post-treatment with the CFD inhibitor:

(a) hemoglobin increase of 2.0 g/dL or greater compared to the subject's baseline hemoglobin level;

(b) transfusion independence; and/or

(c) FACIT Fatigue Scale Score increase of 10 points or greater (e.g., 10, 11, 12) compared to the subject's baseline FACIT Fatigue Scale Score.

In some embodiments, the methods further comprise determining the subject's hemoglobin level, transfusion status, and/or FACIT Fatigue Scale Score at baseline and 12 and/or 24 weeks post-treatment, wherein (a) a hemoglobin increase of 2.0 g/dL or greater compared to the subject's baseline hemoglobin level; (b) transfusion independence; and/or (c) a FACIT Fatigue Scale Score increase of 10 points or greater (e.g., 10, 11, 12) compared to the subject's baseline FACIT Fatigue Scale Score is indicative of treatment.

In some embodiments, the methods involve treating a subject having PNH who previously exhibited an inadequate response to an anti-C5 antibody therapy (e.g., SOLIRIS®, ULTOMIRIS®, 7086 antibody, 8110 antibody, 305LO5 antibody, SKY59 antibody, or REGN3918 antibody). In some embodiments, the subject having PNH previously exhibited an inadequate response to SOLIRIS®. In some embodiments, the subject having PNH previously exhibited an inadequate response to SOLIRIS® at an approved dose or higher for ≥24 weeks (e.g., 24, 25, 26, 27, 28, 29, 30, 31, 32, 33, 34, 35, 36, 37, 38, 39, 40 or more weeks) without change in regimen ≤8 weeks. In some embodiments, the subject having PNH previously exhibited an inadequate response to ULTOMIRIS®.

In some embodiments, the inadequate response by the subject was transfusion dependence (e.g., ≥1 red blood cell (RBC) transfusion ≤12 weeks prior to screening). In some embodiments, the inadequate response by the subject was anemia (e.g., hemoglobin <10 g/dl). In some embodiments, the inadequate response by the subject was transfusion dependence and anemia.

In some embodiments, the subject exhibits one or more clinical improvements after being treated according to the methods described herein. For example, in one embodiment, the subject exhibits a hemoglobin increase of 2.0 g/dL or greater after treatment compared to the subject's baseline hemoglobin level. In other embodiments, the subject exhibits a hemoglobin increase of 2.0 g/dL or greater after 12, 13, 14, 15, 16, 17, 18, 19, 20, 21, 22, 23, 24, 25, 26, 27, 28, 29, or 30 weeks of treatment compared to the subject's baseline hemoglobin level. In other embodiments, the subject exhibits a hemoglobin increase of 2.0 g/dL or greater after 24 weeks of treatment compared to the subject's baseline hemoglobin level. In other embodiments, the subject exhibits transfusion independence after treatment. In other embodiments, the subject exhibits transfusion independence after 12, 13, 14, 15, 16, 17, 18, 19, 20, 21, 22, 23, 24, 25, 26, 27, 28, 29, or 30 weeks of treatment. In other embodiments, the subject exhibits transfusion independence after 24 weeks of treatment. In other embodiments, the subject exhibits transfusion avoidance after treatment. In other embodiments, the subject exhibits transfusion avoidance after 12, 13, 14, 15, 16, 17, 18, 19, 20, 21, 22, 23, 24, 25, 26, 27, 28, 29, or 30 weeks of treatment. In other embodiments, the subject exhibits transfusion avoidance after 12 weeks of treatment. In other embodiments, the subject exhibits a FACIT Fatigue Scale Score increase of 10 points or greater (e.g., 10, 11, 12) after treatment compared to the subject's baseline FACIT Fatigue Scale Score. In other embodiments, the subject exhibits a FACIT Fatigue Scale Score increase of 11 points or greater after treatment compared to the subject's baseline FACIT Fatigue Scale Score. In other embodiments, the subject exhibits a FACIT Fatigue Scale Score increase of 10 points or greater after 12, 13, 14, 15, 16, 17, 18, 19, 20, 21, 22, 23, 24, 25, 26, 27, 28, 29, or 30 weeks of treatment. In other embodiments, the subject exhibits a FACIT Fatigue Scale Score increase of 11 points or greater after 12, 13, 14, 15, 16, 17, 18, 19, 20, 21, 22, 23, 24, 25, 26, 27, 28, 29, or 30 weeks of treatment. In other embodiments, the subject exhibits a FACIT Fatigue Scale Score increase of 10 points or greater after 12 weeks of treatment. In other embodiments, the subject exhibits a FACIT Fatigue Scale Score increase of 11 points or greater after 12 weeks of treatment. In other embodiments, the subject exhibits a FACIT Fatigue Scale Score increase of 10 points or greater after 24 weeks of treatment. In other embodiments, the subject exhibits a FACIT Fatigue Scale Score increase of 11 points or greater after 24 weeks of treatment.

In other embodiments, the subject exhibits a hemoglobin increase compared to the subject's baseline hemoglobin level and transfusion independence or transfusion avoidance after 12 weeks of treatment. In other embodiments, the subject exhibits a hemoglobin increase of 2.0 g/dL compared to the subject's baseline hemoglobin level and transfusion independence, after treatment (e.g., after 12, 13, 14, 15, 16, 17, 18, 19, 20, 21, 22, 23, 24, 25, 26, 27, 28, 29, or 30 weeks of treatment). In other embodiments, the subject exhibits a hemoglobin increase of 2.0 g/dL compared to the subject's baseline hemoglobin level and transfusion independence or transfusion avoidance, after 12 weeks treatment. In other embodiments, the subject exhibits a hemoglobin increase of 2.0 g/dL compared to the subject's baseline hemoglobin level and transfusion independence or transfusion avoidance, after 24 weeks treatment.

In other embodiments, the subject exhibits a hemoglobin increase of compared to the subject's baseline hemoglobin level and a FACIT Fatigue Scale Score increase of 10 points or greater, after 12 weeks of treatment. In other embodiments, the subject exhibits a hemoglobin increase of 2.0 g/dL compared to the subject's baseline hemoglobin level and a FACIT Fatigue Scale Score increase of 10 points or greater, after treatment (e.g., after 12, 13, 14, 15, 16, 17, 18, 19, 20, 21, 22, 23, 24, 25, 26, 27, 28, 29, or 30 weeks of treatment). In other embodiments, the subject exhibits a hemoglobin increase of 2.0 g/dL compared to the subject's baseline hemoglobin level and a FACIT Fatigue Scale Score increase of 11 points or greater, after treatment (e.g., after 12, 13, 14, 15, 16, 17, 18, 19, 20, 21, 22, 23, 24, 25, 26, 27, 28, 29, or 30 weeks of treatment). In other embodiments, the subject exhibits a hemoglobin increase of 2.0 g/dL compared to the subject's baseline hemoglobin level and a FACIT Fatigue Scale Score increase of 10 points or greater, after 12 weeks treatment. In other embodiments, the subject exhibits a hemoglobin increase of 2.0 g/dL compared to the subject's baseline hemoglobin level and a FACIT Fatigue Scale Score increase of 10 points or greater, after 24 weeks treatment. In other embodiments, the subject exhibits a hemoglobin increase of 2.0 g/dL compared to the subject's baseline hemoglobin level and a FACIT Fatigue Scale Score increase of 11 points or greater, after 24 weeks treatment.

In other embodiments, the subject exhibits transfusion independence or transfusion avoidance and a FACIT Fatigue Scale Score increase of 10 points or greater, after 12 weeks of treatment. In other embodiments, the subject exhibits transfusion independence or transfusion avoidance and a FACIT Fatigue Scale Score increase of 10 points or greater, after treatment (e.g., after 12, 13, 14, 15, 16, 17, 18, 19, 20, 21, 22, 23, 24, 25, 26, 27, 28, 29, or 30 weeks of treatment). In other embodiments, the subject exhibits transfusion independence and a FACIT Fatigue Scale Score increase of 11 points or greater, after treatment (e.g., after 12, 13, 14, 15, 16, 17, 18, 19, 20, 21, 22, 23, 24, 25, 26, 27, 28, 29, or 30 weeks of treatment). In other embodiments, the subject exhibits transfusion independence and a FACIT Fatigue Scale Score increase of 10 points or greater, after 24 weeks treatment. In other embodiments, the subject exhibits transfusion independence and a FACIT Fatigue Scale Score increase of 11 points or greater, after 24 weeks treatment

In other embodiments, the subject exhibits a hemoglobin increase of 2.0 g/dL compared to the subject's baseline hemoglobin level, transfusion independence or transfusion avoidance, and a FACIT Fatigue Scale Score increase of 10 points or greater, after treatment (e.g., after 12, 13, 14, 15, 16, 17, 18, 19, 20, 21, 22, 23, 24, 25, 26, 27, 28, 29, or 30 weeks of treatment). In other embodiments, the subject exhibits a hemoglobin increase of 2.0 g/dL compared to the subject's baseline hemoglobin level, transfusion independence or transfusion avoidance, and a FACIT Fatigue Scale Score increase of 11 points or greater, after treatment (e.g., after 12, 13, 14, 15, 16, 17, 18, 19, 20, 21, 22, 23, 24, 25, 26, 27, 28, 29, or 30 weeks of treatment). In other embodiments, the subject exhibits a hemoglobin increase of 2.0 g/dL compared to the subject's baseline hemoglobin level, transfusion independence or transfusion independence, and a FACIT Fatigue Scale Score increase of 10 points or greater, after 24 weeks treatment. In other embodiments, the subject exhibits a hemoglobin increase of 2.0 g/dL compared to the subject's baseline hemoglobin level, transfusion independence or transfusion avoidance, and a FACIT Fatigue Scale Score increase of 11 points or greater, after 24 weeks treatment.

In some embodiments, the method further comprises determining the subject's hemoglobin level, transfusion status, and/or FACIT Fatigue Scale Score at baseline and 12 and/or 24 weeks post-treatment, wherein (a) a hemoglobin increase of 2.0 g/dL or greater compared to the subject's baseline hemoglobin level; (b) transfusion independence; and/or (c) a FACIT Fatigue Scale Score increase of 10 points or greater compared to the subject's baseline FACIT Fatigue Scale Score, is indicative of treatment.

Any suitable CFD inhibitor can be used in the methods described herein. In some embodiments, the CFD inhibitor is a small molecule inhibitor, a nucleotide, a peptide, a protein, a peptide mimetic, an aptamer, or any other molecule that binds to Factor D. In other embodiments, the CFD inhibitor is a nucleotide selected from the group consisting of a DNA, an RNA, an shRNA, an miRNA, an siRNA, and an antisense DNA. In other embodiments, the CFD inhibitor is an antibody, or antigen-binding fragment thereof, that binds to Factor D. In other embodiments, the CFD inhibitor comprises:

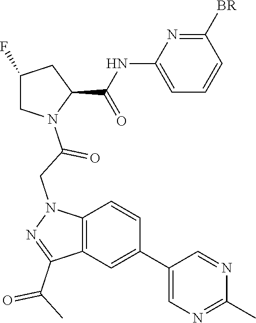

or a pharmaceutically acceptable salt thereof.

An exemplary CFD inhibitor is danicopan.

Other exemplary CFD inhibitors include Compounds 1-7 as set forth in Maibaum, J., et al. (Nature Chemical Biology, volume 12, pages 1105-1110 (2016)). Accordingly, in one embodiment, the CFD inhibitor comprises:

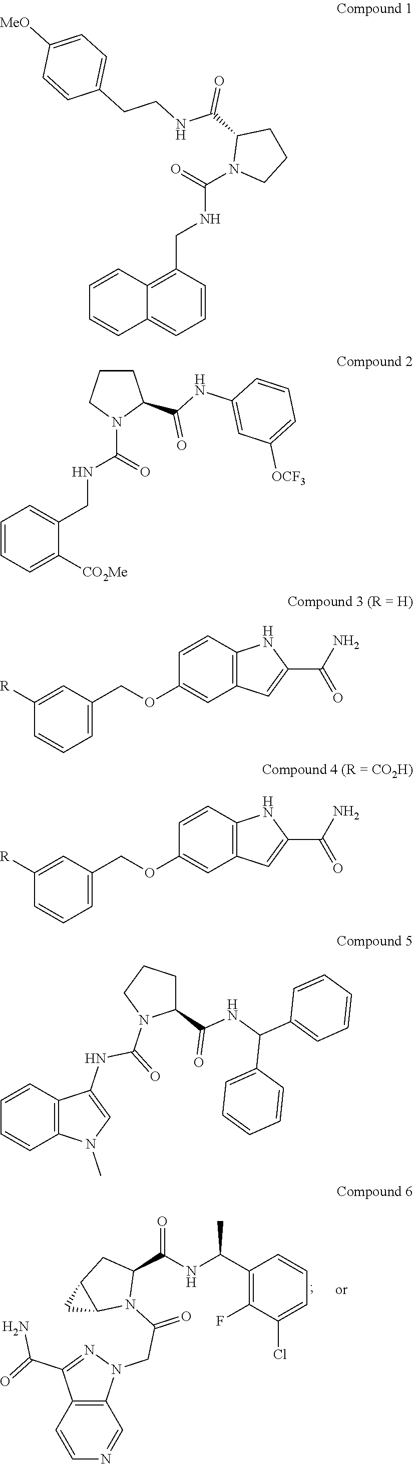

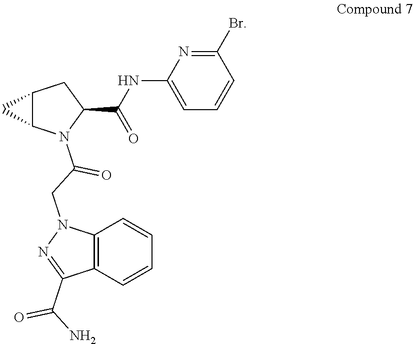 [SMILES: NC(=O)c1nn(CC(=O)N2[C@@H]3C[C@@H]3C[C@H]2C(=O)Nc2cccc(Br)n2)c2ccccc12]

Another exemplary CFD inhibitor is lampalizumab (also referred to as “FCFD4 14S” and “aFD”), described in WO2015168468 and U.S. Pat. No. 10,407,510. Additional exemplary CFD inhibitors include the anti-factor D antibodies described in US20190359699, including mAb 11-8A1, mAb 1F10-5, and variants thereof, the teachings and particular CFD inhibitors disclosed therein, which are all expressly incorporated herein by reference.

Further exemplary CFD inhibitors include the fused bicyclic ring compounds described in U.S. Pat. No. 6,653,340 (including the CFD inhibitor BCX1470 and the compounds disclosed in Examples 1-20), as well as the particular CFD inhibitors described in US 20080269318, including BCX-1470, WO2012/093101 (see, e.g., U.S. Pat. No. 9,085,555), WO2014/002057, WO2014/009833 (see, e.g., U.S. Pat. No. 9,550,755), WO2014/002051 (see, e.g., U.S. Pat. No. 9,815,819), WO2014/002052, WO2014/002053, WO2014/002054, WO2014/002058 (see, e.g., U.S. Pat. No. 9,487,483), WO2014/002059, and WO2014/005150, the teachings and CFD inhibitors disclosed therein, which are all expressly incorporated herein by reference.

Any suitable anti-C5 antibody, or antigen binding fragment thereof, can be used in the methods described herein. In some embodiments, the anti-C5 antibody, or antigen binding fragment thereof, is a human antibody, a humanized antibody, a bispecific antibody, a chimeric antibody, a Fab, a Fab′2, a ScFv, a SMIP, an Affibody®, a nanobody, or a domain antibody.

An exemplary anti-C5 antibody is SOLIRIS® (also known as eculizumab). SOLIRIS® is an anti-C5 antibody comprising heavy and light chains having sequences shown in SEQ ID NO: 10 and 11, respectively, or antigen binding fragments and variants thereof. In some embodiments, the anti-C5 antibody, comprises the CDR1, CDR2, and CDR3 domains of the VH region of SOLIRIS® having the sequence set forth in SEQ ID NO: 7, and the CDR1, CDR2 and CDR3 domains of the VL region of SOLIRIS® having the sequence set forth in SEQ ID NO: 8. In other embodiments, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In other embodiments, the antibody comprises VH and VL regions having the amino acid sequences set forth in SEQ ID NO: 7 and SEQ ID NO: 8, respectively.

Another exemplary anti-C5 antibody is ULTOMIRIS® (ravulizumab) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ULTOMIRIS®. Accordingly, in one embodiment, the antibody comprises the CDR1, CDR2 and CDR3 domains of the heavy chain variable (VH) region of ULTOMIRIS® having the sequence shown in SEQ ID NO:12, and the CDR1, CDR2 and CDR3 domains of the light chain variable (VL) region of ULTOMIRIS® having the sequence shown in SEQ ID NO:8. In other embodiments, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively. In other embodiments, the antibody comprises VH and VL regions having the amino acid sequences set forth in SEQ ID NO:12 and SEQ ID NO:8, respectively. In other embodiments, the antibody comprises a heavy chain constant region as set forth in SEQ ID NO:13.

In other embodiments, the antibody comprises a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In other embodiments, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In other embodiments, the anti-C5 antibody comprises the heavy and light chain CDRs or variable regions of the BNJ421 antibody (described in PCT/US2015/019225 and U.S. Pat. No. 9,079,949). In other embodiments, the anti-C5 antibody comprises the heavy and light chain CDRs or variable regions of the 7086 antibody (see U.S. Pat. Nos. 8,241,628 and 8,883,158). In some embodiments, the anti-C5 antibody, or antigen-binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In some embodiments, the anti-C5 antibody, or antigen-binding fragment thereof, comprises a heavy chain variable region comprising the sequence set forth in SEQ ID NO:27 and a light chain variable region having the sequence set forth in SEQ ID NO:28.

In some embodiments, the anti-C5 antibody comprises the heavy and light chain CDRs or variable regions of the 8110 antibody (see U.S. Pat. Nos. 8,241,628 and 8,883,158). In some embodiments, the anti-C5 antibody, or antigen-binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In some embodiments, the anti-C5 antibody, or antigen-binding fragment thereof, comprises a heavy chain variable region comprising the sequence set forth in SEQ ID NO:35 and a light chain variable region having the sequence set forth in SEQ ID NO:36.

In some embodiments, the anti-C5 antibody comprises the heavy and light chain CDRs or variable regions of the 305LO5 antibody (see US2016/0176954A1). In some embodiments, the anti-C5 antibody, or antigen-binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In some embodiments, the anti-C5 antibody, or antigen-binding fragment thereof, comprises a heavy chain variable region comprising the sequence set forth in SEQ ID NO:43 and a light chain variable region having the sequence set forth in SEQ ID NO:44.

In some embodiments, the anti-C5 antibody comprises the heavy and light chain CDRs or variable regions of the SKY59 antibody. In some embodiments, the anti-C5 antibody, or antigen-binding fragment thereof, comprises a heavy chain comprising the sequence set forth in SEQ ID NO: 45 and a light chain comprising the sequence set forth in SEQ ID NO: 46.

In some embodiments, the anti-C5 antibody comprises the heavy and light chain variable regions or heavy and light chains of the REGN3918 antibody (see US20170355757). In some embodiments, the anti-C5 antibody, or antigen-binding fragment thereof, comprises a heavy chain variable region sequence set forth in SEQ ID NO: 47 and a light chain variable region comprising the sequence set forth in SEQ ID NO: 48. In some embodiments, the anti-C5 antibody, or antigen-binding fragment thereof, comprises a heavy chain sequence set forth in SEQ ID NO: 49 and a light chain sequence set forth in SEQ ID NO: 50.

In other embodiments, the antibody competes for binding with, and/or binds to the same epitope on C5 as any of the above-mentioned antibodies. In other embodiments, the antibody has at least about 90% variable region amino acid sequence identity to any of the above-mentioned antibodies (e.g., at least about 90%, 95% or 99% variable region identity with SEQ ID NO:12 or SEQ ID NO:8).

In other embodiments, the antibody binds to human C5 at pH 7.4 and 25° C. with an affinity dissociation constant (K_{D}) that is in the range 0.1 nM≤K_{D}≤1 nM. In other embodiments, the antibody binds to human C5 at pH 6.0 and 25° C. with a K_{D}≥10 nM. In yet another embodiment, the [(K_{D} of the antibody or antigen-binding fragment thereof for human C5 at pH 6.0 and at 25 C)/(K_{D} of the antibody or antigen-binding fragment thereof for human C5 at pH 7.4 and at 25 C)] of the antibody is greater than 25.

In some embodiments, the CFD inhibitor (e.g., danicopan) is administered (or is for administration) according to a particular clinical dosage regimen (e.g., at a particular dose amount and according to a specific dosing schedule). In some embodiments, the CFD inhibitor is administered orally to the subject. In some embodiments, the CFD inhibitor is administered orally three times daily (TID) to the subject. In some embodiments, the CFD inhibitor is administered orally at a dose of between about 50 mg to 300 mg to the subject. In some embodiments, the CFD inhibitor is administered orally at a dose of about 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the CFD inhibitor is administered orally at a dose of about 100 mg. In some embodiments, the CFD inhibitor is administered orally at a dose of about 100 mg TID. In some embodiments, the CFD inhibitor is administered orally at a dose of about 150 mg. In some embodiments, the CFD inhibitor is administered orally at a dose of about 150 mg TID. In some embodiments, the CFD inhibitor is administered orally at a dose of about 200 mg. In some embodiments, the CFD inhibitor is administered orally at a dose of about 200 mg TID.

In some embodiments, the CFD inhibitor is administered for 4 weeks or more (e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks or more). In some embodiments, the CFD inhibitor is administered for 24 weeks. In some embodiments, the CFD inhibitor is administered for 9 months, 12 months, 15 months, 20 months, 24 months or longer. In some embodiments, the CFD inhibitor is administered for 1, 2, 3, 4, 5, 6 or more years.

In some embodiments, the anti-C5 antibody, or antigen binding fragment thereof (e.g., SOLIRIS® or ULTOMIRIS®), is administered (or is for administration) according to a particular clinical dosage regimen (e.g., at a particular dose amount and according to a specific dosing schedule). The anti-C5 antibodies, or antigen binding fragments thereof, can be administered to a patient by any suitable means. In some embodiments, the anti-C5 antibody, or antigen binding fragment thereof, is administered intravenously to the subject.

In some embodiments, the dose of the anti-C5 antibody, or antigen binding fragment thereof, is a fixed dose. For example, in some embodiments, the anti-C5 antibody, or antigen binding fragment thereof, is administered to the subject at a dose of 600 mg weekly. In some embodiments, the anti-C5 antibody, or antigen binding fragment thereof, is administered to the subject at a dose of 900 mg every two weeks.

In some embodiments, the anti-C5 antibody, or antigen binding fragment thereof, is administered to the subject at a dose of 600 mg weekly for four doses, followed by a dose of 900 mg at Week 5 and then at a dose of 900 mg every 2 weeks thereafter. In some embodiments, SOLIRIS® is administered to the subject (e.g., an adult subject) at a dose of 600 mg weekly for four doses, followed by a dose of 900 mg at Week 5 and then at a dose of 900 mg every 2 weeks thereafter.

In some embodiments, the anti-C5 antibody, or antigen binding fragment thereof, is administered to a subject less than 18 years of age at a dose of 900 mg weekly for four doses to a subject weighing 40 kg and over, followed by a dose of 1200 mg at Week 5 and then at a dose of 1200 mg every two weeks thereafter. In some embodiments, SOLIRIS® is administered to a subject less than 18 years of age at a dose of 900 mg weekly for four doses to a subject weighing 40 kg and over, followed by a dose of 1200 mg at Week 5 and then at a dose of 1200 mg every two weeks thereafter.

In some embodiments, the anti-C5 antibody, or antigen binding fragment thereof, is administered to a subject less than 18 years of age at a dose of 600 mg weekly for two doses to a subject weighing 30 kg to less than 40 kg, followed by a dose of 900 mg at Week 3 and then at a dose of 900 mg every two weeks thereafter. In some embodiments, SOLIRIS® is administered to a subject less than 18 years of age at a dose of 600 mg weekly for two doses to a subject weighing 30 kg to less than 40 kg, followed by a dose of 900 mg at Week 3 and then at a dose of 900 mg every two weeks thereafter.

In some embodiments, the anti-C5 antibody, or antigen binding fragment thereof, is administered to a subject less than 18 years of age at a dose of 600 mg weekly for two doses to a subject weighing 20 kg to less than 30 kg, followed by a dose of 600 mg at Week 3 and then at a dose of 600 mg every two weeks thereafter. In some embodiments, SOLIRIS® is administered to a subject less than 18 years of age at a dose of 600 mg weekly for two doses to a subject weighing 20 kg to less than 30 kg, followed by a dose of 600 mg at Week 3 and then at a dose of 600 mg every two weeks thereafter.

In some embodiments, the anti-C5 antibody, or antigen binding fragment thereof, is administered to a subject less than 18 years of age at a dose of 600 mg weekly for one dose to a subject weighing 10 kg to less than 20 kg, followed by a dose of 300 mg at Week 3 and then at a dose of 300 mg every two weeks thereafter. In some embodiments, SOLIRIS® is administered to a subject less than 18 years of age at a dose of 600 mg weekly for one dose to a subject weighing 10 kg to less than 20 kg, followed by a dose of 300 mg at Week 3 and then at a dose of 300 mg every two weeks thereafter.

In some embodiments, the anti-C5 antibody, or antigen binding fragment thereof, is administered to a subject less than 18 years of age at a dose of 300 mg weekly for one dose to a subject weighing 5 kg to less than 10 kg, followed by a dose of 300 mg at Week 2 and then at a dose of 300 mg every three weeks thereafter. In some embodiments, SOLIRIS® is administered to a subject less than 18 years of age at a dose of 300 mg weekly for one dose to a subject weighing 5 kg to less than 10 kg, followed by a dose of 300 mg at Week 2 and then at a dose of 300 mg every three weeks thereafter.

In some embodiments, the dose of the anti-C5 antibody, or antigen binding fragment thereof, is based on the weight of the patient. In some embodiments, for example, 300 mg of the anti-C5 antibody, or antigen binding fragment thereof, is administered to a patient weighing ≥5 to <10 kg. In some embodiments, 600 mg of the anti-C5 antibody, or antigen binding fragment thereof, is administered to a patient weighing ≥10 to <20 kg. In some embodiments, 900 mg or 2100 mg of the anti-C5 antibody, or antigen binding fragment thereof, is administered to a patient weighing ≥20 to <30 kg. In some embodiments, 1200 mg or 2700 mg of the anti-C5 antibody, or antigen binding fragment thereof, is administered to a patient weighing ≥30 to <40 kg. In some embodiments, 2400 mg or 3000 mg of the anti-C5 antibody, or antigen binding fragment thereof, is administered to a patient weighing ≥40 to <60 kg. In some embodiments, 2700 mg or 3300 mg of the anti-C5 antibody, or antigen binding fragment thereof, is administered to a patient weighing ≥60 to <100 kg. In some embodiments, 3000 mg or 3600 mg of the anti-C5 antibody, or antigen binding fragment thereof, is administered to a patient weighing ≥100 kg. In certain embodiments, dosage regimens are adjusted to provide the optimum desired response (e.g., an effective response).

In some embodiments, the anti-C5 antibody, or antigen binding fragment thereof, is administered:
(a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and
(b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In some embodiments, ULTOMIRIS®, is administered:
(a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and
(b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In some embodiments, the anti-C5 antibody, or antigen binding fragment thereof, is administered to a patient weighing ≥40 to <60 kg:
(a) once on Day 1 of the administration cycle at a dose of 2400 mg; and
(b) on Day 15 of the administration cycle and every eight weeks thereafter at a dose of 3000 mg.

In some embodiments, ULTOMIRIS® is administered to a patient weighing ≥40 to <60 kg:
(a) once on Day 1 of the administration cycle at a dose of 2400 mg; and
(b) on Day 15 of the administration cycle and every eight weeks thereafter at a dose of 3000 mg.

In some embodiments, the anti-C5 antibody, or antigen binding fragment thereof, is administered to a patient weighing ≥60 to <100 kg:
(a) once on Day 1 of the administration cycle at a dose of 2700 mg; and
(b) on Day 15 of the administration cycle and every eight weeks thereafter at a dose of 3300 mg.

In some embodiments, ULTOMIRIS® is administered to a patient weighing ≥60 to <100 kg:
(a) once on Day 1 of the administration cycle at a dose of 2700 mg; and
(b) on Day 15 of the administration cycle and every eight weeks thereafter at a dose of 3300 mg.

In some embodiments, the anti-C5 antibody, or antigen binding fragment thereof, is administered to a patient weighing ≥100 kg:
(a) once on Day 1 of the administration cycle at a dose of 3000 mg; and
(b) on Day 15 of the administration cycle and every eight weeks thereafter at a dose of 3600 mg.

In some embodiments, ULTOMIRIS® is administered to a patient weighing ≥100 kg:
(a) once on Day 1 of the administration cycle at a dose of 3000 mg; and
(b) on Day 15 of the administration cycle and every eight weeks thereafter at a dose of 3600 mg.

In some embodiments, the anti-C5 antibody, or antigen binding fragment thereof, is administered to a subject less than 18 years of age:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and
(b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In some embodiments, the anti-C5 antibody, or antigen binding fragment thereof, is administered to a patient weighing ≥5 to <10 kg: (a) once on Day 1 at a dose of 600 mg; and (b) on Day 15 and every four weeks thereafter at a dose of 300 mg. In some embodiments, ULTOMIRIS® is administered to a patient weighing ≥5 to <10 kg: (a) once on Day 1 at a dose of 600 mg; and (b) on Day 15 and every four weeks thereafter at a dose of 300 mg.

In some embodiments, the anti-C5 antibody, or antigen binding fragment thereof, is administered to a patient weighing ≥10 to <20 kg: (a) once on Day 1 at a dose of 600 mg; and (b) on Day 15 and every four weeks thereafter at a dose of 600 mg. In some embodiments, the anti-C5 antibody is administered to a patient weighing ≥10 to <20 kg: (a) once on Day 1 at a dose of 600 mg; and (b) on Day 15 and every four weeks thereafter at a dose of 600 mg.

In some embodiments, the anti-C5 antibody, or antigen binding fragment thereof, is administered to a patient weighing ≥20 to <30 kg: (a) once on Day 1 at a dose of 900 mg; and (b) on Day 15 and every eight weeks thereafter at a dose of 2100 mg. In some embodiments, ULTOMIRIS® is administered to a patient weighing ≥20 to <30 kg: (a) once on Day 1 at a dose of 900 mg; and (b) on Day 15 and every eight weeks thereafter at a dose of 2100 mg.

In some embodiments, the anti-C5 antibody, or antigen binding fragment thereof, is administered to a patient weighing ≥30 to <40 kg: (a) once on Day 1 at a dose of 1200 mg; and (b) on Day 15 and every eight weeks thereafter at a dose of 2700 mg. In some embodiments, ULTOMIRIS® is administered to a patient weighing ≥30 to <40 kg: (a) once on Day 1 at a dose of 1200 mg; and (b) on Day 15 and every eight weeks thereafter at a dose of 2700 mg.

In some embodiments, the anti-C5 antibody, or antigen binding fragment thereof, is administered to a patient weighing ≥40 to <60 kg: (a) once on Day 1 at a dose of 2400 mg; and (b) on Day 15 and every eight weeks thereafter at a dose of 3000 mg. In some embodiments, ULTOMIRIS® is administered to a patient weighing ≥40 to <60 kg: (a) once on Day 1 at a dose of 2400 mg; and (b) on Day 15 and every eight weeks thereafter at a dose of 3000 mg.

In some embodiments, the anti-C5 antibody, or antigen binding fragment thereof, is administered to a patient weighing ≥60 to <100 kg: (a) once on Day 1 at a dose of 2700 mg; and (b) on Day 15 and every eight weeks thereafter at a dose of 3300 mg. In some embodiments, ULTOMIRIS® is administered to a patient weighing ≥60 to <100 kg: (a) once on Day 1 at a dose of 2700 mg; and (b) on Day 15 and every eight weeks thereafter at a dose of 3300 mg.

In some embodiments, the anti-C5 antibody, or antigen binding fragment thereof, is administered to a patient weighing ≥100 kg: (a) once on Day 1 at a dose of 3000 mg; and (b) on Day 15 and every eight weeks thereafter at a dose of 3600 mg. In some embodiments, ULTOMIRIS® is administered to a patient weighing ≥100 kg: (a) once on Day 1 at a dose of 3000 mg; and (b) on Day 15 and every eight weeks thereafter at a dose of 3600 mg.

In another aspect, the treatment regimens described are sufficient to maintain particular serum trough concentrations of the anti-C5 antibody, or antigen binding fragment thereof. In one embodiment, for example, the treatment regimen maintains a serum trough concentration of the anti-C5 antibody, or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In some embodiments, the treatment regimen maintains a serum trough concentration of the anti-C5 antibody, or antigen binding fragment thereof of 100 μg/mL or greater, 150 μg/mL or greater, 200 μg/mL or greater, 250 μg/mL or greater, or 300 μg/mL or greater. In some embodiments, the treatment maintains a serum trough concentration of the anti-C5 antibody, or antigen binding fragment thereof of between 100 μg/mL and 200 μg/mL. In some embodiments, the treatment maintains a serum trough concentration of the anti-C5 antibody, or antigen binding fragment thereof of about 175 μg/mL.

In some embodiments, to obtain an effective response, the anti-C5 antibody, or antigen binding fragment thereof, is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg or 260 μg of antibody per milliliter of the patient's blood. In some embodiments, the anti-C5 antibody is administered to the patient in an amount and with a frequency to maintain between 50 μg and 250 μg of antibody per milliliter of the patient's blood. In some embodiments, the anti-C5 antibody is administered to the patient in an amount and with a frequency to maintain between 100 μg and 200 μg of antibody per milliliter of the patient's blood. In some embodiments, the anti-C5 antibody is administered to the patient in an amount and with a frequency to maintain about 175 μg of antibody per milliliter of the patient's blood.

The efficacy of the treatment methods provided herein can be assessed using any suitable means. In some embodiments, the treatment results in a shift toward normal levels of bilirubin (e.g., from about 0.2-1.2 mg/dL). In some embodiments, the treatment results in a reduction in reticulocytes compared to baseline (e.g., a 2, 3, 4 or 5-fold reduction). In some embodiment, the treatment results in an increase in PNH specific red blood cell clone size compared to baseline (e.g., a 2, 3, 4, or 5-fold increase). In some embodiments, the treatment results in a decrease in PNH erythrocytes opsonized with C3 fragment compared to baseline (e.g., a 2, 3, 4, or 5-fold reduction). In some embodiments, the treatment produces a reduction in the need for blood transfusions compared to baseline. In some embodiments, the treatment results in terminal complement inhibition. In some embodiments, the treatment produces at least one therapeutic effect selected from the group consisting of: a reduction or cessation in abdominal pain, dyspnea, dysphagia, chest pain and erectile dysfunction compared to baseline. In some embodiments, the treatment produces a shift toward normal levels of at least one or more hemolysis-related hematologic biomarkers selected from the group consisting of: free hemoglobin, haptoglobin, reticulocyte count, PNH red blood cell (RBC) clone and/or D-dimer. In some embodiments, the treatment produces a reduction in major adverse vascular events (MAVEs). In some embodiments, the treatment produces a shift toward normal levels of estimated glomerular filtration rate (eGFR) or spot urine:albumin:creatinine and plasma brain natriuretic peptide (BNP). In some embodiments, the treatment produces a change from baseline in quality of life, assessed via version 4 and the European Organisation for Research and Treatment of Cancer, Quality of Life Questionnaire-Core 30 Scale compared to baseline.

In some embodiments, LDH levels are used to evaluate responsiveness to a therapy (e.g., a reduction of hemolysis as assessed by LDH levels is indicative of an improvement in at least one sign of PNH). In some embodiments, patients treated according to the disclosed methods experience reductions in LDH levels to near normal levels or to within 10%, 20%, 30%, 40% or within 50% below what is considered the normal level (e.g., within 105-333 IU/L (international units per liter). In some embodiments, the patient's LDH levels are normalized throughout maintenance period of treatment. In some embodiments, the treated patient's LDH levels are normalized at least at least 95% of the time while on the maintenance period of treatment. In some embodiments, the treated patient's LDH levels are normalized at least at least 90%, 85% or 80% of the time while on the maintenance period of treatment. In some embodiments, the patient's LDH levels are ≥1.5 fold above the upper limit of normal (LDH≥1.5×ULN) prior to initiating treatment.

In one embodiment, a method for PNH in a subject who had an inadequate response to prior treatment with SOLIRIS® (eculizumab) is provided, the method comprising:

administering to the subject a therapeutically effective amount of danicopan in combination with a therapeutically effective amount of SOLIRIS® (eculizumab),

wherein the inadequate response by the subject was transfusion dependence and/or anemia; and

wherein danicopan is administered to the subject orally at a dose of 100 mg, 150 mg, or 200 mg TID to the subject;

wherein SOLIRIS® (eculizumab) is administered intravenously to the subject at a dose of 600 mg weekly for four doses, followed by a dose of 900 mg at Week 5 and then at a dose of 900 mg every 2 weeks thereafter; and

wherein the subject exhibits one or more of the following clinical improvements 12 and/or 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater compared to the subject's baseline FACIT Fatigue Scale Score.

In another embodiment, a method for treating PNH in a subject who had an inadequate response to prior treatment with SOLIRIS® (eculizumab) is provided, the method comprising: administering to the subject a therapeutically effective amount of danicopan in combination with a therapeutically effective amount of SOLIRIS® (eculizumab),

wherein the inadequate response by the subject was transfusion dependence and/or anemia; and

wherein danicopan is administered to the subject orally at a dose of 100 mg, 150 mg, or 200 mg TID to the subject;

wherein SOLIRIS® (eculizumab) is administered intravenously to a subject less than 18 years of age:
(a) at a dose of 900 mg weekly for four doses to a subject weighing 40 kg and over, followed by a dose of 1200 mg at Week 5 and then at a dose of 1200 mg every two weeks thereafter;
(b) at a dose of 600 mg weekly for two doses to a subject weighing 30 kg to less than 40 kg, followed by a dose of 900 mg at Week 3 and then at a dose of 900 mg every two weeks thereafter;
(c) at a dose of 600 mg weekly for two doses to a subject weighing 20 kg to less than 30 kg, followed by a dose of 600 mg at Week 3 and then at a dose of 600 mg every two weeks thereafter;
(d) at a dose of 600 mg weekly for one dose to a subject weighing 10 kg to less than 20 kg, followed by a dose of 300 mg at Week 3 and then at a dose of 300 mg every two weeks thereafter; or
(e) at a dose of 300 mg weekly for one dose to a subject weighing 5 kg to less than 10 kg, followed by a dose of 300 mg at Week 2 and then at a dose of 300 mg every three weeks thereafter; and

wherein the subject exhibits one or more of the following clinical improvements 12 and/or 24 weeks post-treatment with the CFD inhibitor:
i. hemoglobin increase of 2.0 g/dL or greater compared to the subject's baseline hemoglobin level;
ii. transfusion independence; and/or
iii. FACIT Fatigue Scale Score increase of 10 points or greater compared to the subject's baseline FACIT Fatigue Scale Score.

In another embodiment, a method for treating PNH in a subject is provided, the method comprising:

administering to the subject a therapeutically effective amount of danicopan in combination with a therapeutically effective amount of SOLIRIS® (eculizumab),

wherein danicopan is administered to the subject orally at a dose of 100 mg, 150 mg, or 200 mg TID to the subject;
wherein SOLIRIS® (eculizumab) is administered intravenously to the subject at a dose of 600 mg weekly for four doses, followed by a dose of 900 mg at Week 5 and then at a dose of 900 mg every 2 weeks thereafter; and
wherein the subject exhibits one or more of the following clinical improvements 12 or 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater compared to the subject's baseline FACIT Fatigue Scale Score.

In another embodiment, a method for treating PNH in a subject less than 18 years of age is provided, the method comprising administering to the subject a therapeutically effective amount of danicopan in combination with a therapeutically effective amount of SOLIRIS® (eculizumab),

wherein danicopan is administered to the subject orally at a dose of 100 mg, 150 mg, or 200 mg TID to the subject;

wherein SOLIRIS® (eculizumab) is administered intravenously:
(a) at a dose of 900 mg weekly for four doses to a subject weighing 40 kg and over, followed by a dose of 1200 mg at Week 5 and then at a dose of 1200 mg every two weeks thereafter;
(b) at a dose of 600 mg weekly for two doses to a subject weighing 30 kg to less than 40 kg, followed by a dose of 900 mg at Week 3 and then at a dose of 900 mg every two weeks thereafter;
(c) at a dose of 600 mg weekly for two doses to a subject weighing 20 kg to less than 30 kg, followed by a dose of 600 mg at Week 3 and then at a dose of 600 mg every two weeks thereafter;
(d) at a dose of 600 mg weekly for one dose to a subject weighing 10 kg to less than 20 kg, followed by a dose of 300 mg at Week 3 and then at a dose of 300 mg every two weeks thereafter; or
(e) at a dose of 300 mg weekly for one dose to a subject weighing 5 kg to less than 10 kg, followed by a dose of 300 mg at Week 2 and then at a dose of 300 mg every three weeks thereafter; and

wherein the subject exhibits one or more of the following clinical improvements 24 weeks post-treatment with the CFD inhibitors:
i. hemoglobin increase of 2.0 g/dL or greater compared to the subject's baseline hemoglobin level;
ii. transfusion independence; and/or
iii. FACIT Fatigue Scale Score increase of 10 points or greater compared to the subject's baseline FACIT Fatigue Scale Score.

In some embodiments, the methods described herein further comprise determining the subject's hemoglobin level, transfusion status, and/or FACIT Fatigue Scale Score at baseline and 12 and/or 24 weeks post-treatment, wherein
(a) a hemoglobin increase of 2.0 g/dL or greater compared to the subject's baseline hemoglobin level;
(b) transfusion independence; and/or
(c) a FACIT Fatigue Scale Score increase of 10 points or greater compared to the subject's baseline FACIT Fatigue Scale Score, is indicative of treatment.

In some embodiments, the disclosure relates to a method for treating PNH in a subject who previously exhibited an inadequate response to a C5 inhibitor, e.g., an anti-C5 antibody therapy, comprising administering to the subject a therapeutically effective amount of an inhibitor of the alternative pathway (AP) of complement selected from the group consisting of:
a) MASP-3 inhibitor (e.g., α-MASP-3 monoclonal antibody (Mab), such as OMS906);
b) Factor D (FD) inhibitor (e.g., anti-FD Mab, such as lampalizumab or a small molecule FD inhibitor, such as danicopan (ACH-4471), or BCX9930);
c) Factor B inhibitor (e.g., LNP023);
d) a compstatin molecule or a derivative thereof (e.g., APL2, APL9, AMY-101);
e) a mini Factor H (e.g., mini FH AMY-201); and
f) a factor H fusion protein (e.g., TT30).

In some embodiments, the disclosure relates to a method for treating PNH in a subject who previously exhibited an inadequate response to a C5 inhibitor, e.g., an anti-C5 antibody therapy, comprising administering to the subject a therapeutically effective amount of danicopan; particularly wherein a pharmaceutical composition comprising about 100 to about 200 mg danicopan is administered to a human subject every 8 hours.

In some embodiments, the disclosure relates to a method for treating PNH in a subject who previously exhibited an inadequate response to a C5 inhibitor selected from the group consisting of:
a) an eculizumab biosimilar (e.g., ABP 959; Elizaria; or SB12);
b) Nomacopan (Coversin; rVA576);
c) ULTOMIRIS® (ravulizumab);
d) Tesidolumab (LFG316);
e) Pozelimab; and
f) Crovalimab (SKY059).

Further provided herein are kits for treating PNH. In some embodiments, the kit comprises: (a) a dose of a complement factor D (CFD) inhibitor and (b) instructions for using the CFD, in any of the methods described herein. In some embodiments, the kit comprises: (a) a dose of a complement factor D (CFD) inhibitor, (b) a dose of an anti-C5 antibody; and (c) instructions for using the CFD and anti-C5 antibody, in any of the methods described herein. In some embodiments, the CFD is danicopan. In some embodiments, the anti-C5 antibody is SOLIRIS® or ULTOMIRIS®.

### BRIEF DESCRIPTION OF THE DRAWINGS

FIG. 1 is a schematic depicting the dosing regimen for the clinical trial.

FIG. 2 depicts historical and “on treatment” transfusion data for individual patients in the clinical trial. Specifically, FIG. 2 shows per patient transfusion occurrences and units 52 weeks prior to the start of danicopan and during treatment with danicopan.

FIG. 3 is a graph depicting transfusion frequency and unit amount via annualized rates and units.

FIGS. 4A-4D are graphs depicting the effect on complement biomarkers and PNH clone size. Specifically, serum, plasma and whole blood samples were collected at Day 1 prior to dosing danicopan (baseline) and at selected time points during the study course as indicated and were subjected to measurement of CP activity (FIG. 4A), AP activity with AP hemolysis assay (FIG. 4.B), plasma Bb concentration (FIG. 4C) and the clone size of PNH granulocytes, PNH erythrocytes and C3d⁺ PNH erythrocytes (FIG. 4D). Arithmetic mean and standard derivation (SD) were shown for all but the clone size of C3d⁺ PNH erythrocytes where geometric mean was used, and the range was shown at each time point. NHS, normal human serum; BL, baseline; LLN, lower limit normal; ULN, upper limit normal.

### DETAILED DESCRIPTION

### I. Definitions

As used herein, the term “subject” or “patient” is a human patient (e.g., a patient having Paroxysmal Nocturnal Hemoglobinuria (PNH)).

As used herein, the term “pediatric” patient is a human patient under 18 years of age (<18 years of age).

PNH is an acquired hemolytic disorder that occurs most frequently in adults (Brodsky, R., Blood, 126:2459-65, 2015). The disease begins with the clonal expansion of a hematopoietic stem cell that has acquired a somatic mutation in the PIGA gene (Brodsky, R., Blood, 124:2804-11, 2014). Consequently, PNH blood cells lack the glycophosphatidylinositol (GPI) anchor protein and are deficient in the membrane-bound complement inhibitory proteins CD55 and CD59. In the absence of CD55, there is increased deposition of complement protein C3 cleavage products on blood cell membrane surfaces, in turn leading to cleavage of C5 into C5a and C5b. The pathology and clinical presentations in patients with PNH are driven by uncontrolled terminal complement activation.

C5a is a potent anaphylatoxin, chemotactic factor, and cell-activating molecule that mediates multiple pro-inflammatory and pro-thrombotic activities (Matis, L & Rollins, S., Nat. Med., 1:839-42, 1995; Prodinger et al., Complement. In: Paul W E, editor. Fundamental immunology (4th ed). Philadelphia: Lippincott-Raven Publishers; 1999. p. 967-95). C5b recruits the terminal complement components C6, C7, C8 and C9 to form the pro-inflammatory, pro-thrombotic cytolytic pore molecule C5b-9, a process that under normal circumstances would be blocked on the red blood cell (RBC) membrane by CD59. In patients with PNH, however, these final steps proceed unchecked, culminating in hemolysis and the release of free hemoglobin, as well as platelet activation (Hill, A. et al., Blood, 121:4985-96, 2013). The signs and symptoms of PNH can be attributed to chronic, uncontrolled complement C5 cleavage, and release of C5a and C5b-9 leading to RBC hemolysis, which together result in:
release of intracellular free hemoglobin and lactate dehydrogenase (LDH) into circulation as a direct consequence of hemolysis;
irreversible binding to and inactivation of nitric oxide (NO) by hemoglobin, and inhibition of NO synthesis;
vasoconstriction and tissue-bed ischemia due to absence of vasodilatory NO, as well as possible microthrombi manifesting as abdominal pain, dysphagia and erectile dysfunction;
platelet activation; and
a pro-inflammatory and prothrombotic state.

A substantial proportion of patients with PNH experience renal dysfunction and pulmonary hypertension (Hillmen, P. et al., Am. J. Hematol., 85:553-9, 2010 [erratum in Am. J. Hematol., 85:911, 2010]; Hill, A. et al., Br. J. Haematol., 158:409-14, 2012). Patients also experience venous or arterial thrombosis in diverse sites, including the abdomen or central nervous system.

In contrast, children with PNH usually present with nonspecific symptoms related to the underlying bone marrow disorder, such as pallor, fatigue or jaundice, with hemoglobinuria appearing less commonly (Ware, R. et al., N. Engl. J. Med., 325:991-6, 1991). Clinical evaluation in pediatric patients also reveals bone marrow failure syndromes, such as, for example, aplastic anemia and refractory cytopenia (van den Heuvel-Eibrink, M., Paediatr. Drugs, 9:11-6, 2007). Once the bone marrow disorder is resolved in the child or the PNH clone expands (the cause of which is still unknown), the disease eventually evolves into one more typically seen in adults at presentation.

As used herein “anemia” or “anemic” refers to a low number of red blood cells, i.e., hemoglobin <10 g/dl.

As used herein, “hemolysis” refers to the rupture or destruction of red blood cells (RBCs). “Intravascular hemolysis” refers to the lysis of RBCs in the circulation, thereby releasing hemoglobin into the plasma. The resulting fragmented RBCs are called “schistocytes”. “Extravascular hemolysis” refers to the lysis and phagocytosis of RBCs by macrophages in the spleen and liver. Extravascular hemolysis is characterized by spherocytes.

As used herein, “transfusion” refers to an act of transferring blood, blood products, or other fluid into the circulatory system of a subject. A subject that is “transfusion dependent” is a subject who has had ≥1 transfusion (e.g., a red blood cell transfusion)≤12 weeks prior to screening and/or treatment. A subject that is “transfusion independent” is a subject who has gone >12 weeks without a transfusion (e.g., a red blood cell transfusion).

As used herein, “transfusion avoidance” refers to a subject treated according to the methods described herein remaining transfusion-free and not requiring a transfusion through week 12 of treatment. A transfusion (e.g., of packed red blood cells (pRBCs) is required when a subject has a (1) hemoglobin value of less than 6 g/dL regardless of presence of clinical signs or symptoms, or (2) a hemoglobin value of less than 9 g/dL with signs or symptoms of sufficient severity to warrant a transfusion. As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method. Effective treatment may refer to alleviation of at least one symptom of PNH (e.g., pallor, fatigue, jaundice, anemia, cytopenia, abdominal pain, dyspnea, dysphagia, chest pain or erectile dysfunction).

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount of anti-C5 antibody, or antigen binding fragment thereof, clinically proven to alleviate at least one symptom of PNH (e.g., pallor, fatigue, jaundice, anemia, cytopenia, abdominal pain, dyspnea, dysphagia, or chest pain). An effective amount can be administered in one or more administrations.

As used herein, the terms “maintenance” and “maintenance phase” are used interchangeably and refer to the second phase of treatment. In certain embodiments, treatment is continued as long as clinical benefit is observed or until unmanageable toxicity or disease progression occurs.

As used herein, the term “alternate component of complement” refers to a component other than a listed component, e.g., a component other than C5, such as, for example, MASP3, Factor D, Factor B, C3/C5 convertase, and the like.

As used herein, the term “C5 inhibitor” refers, in the broadest sense, to any molecule which inhibits or antagonizes C5, e.g., an antibody selected from (a) eculizumab or a biosimilar thereof, e.g., ABP 959; Elizaria; or SB12; (b) Ravulizumab; (c) Tesidolumab (LFG316); (d) Pozelimab; and (e) Crovalimab (SKY059); or a protein/peptide inhibitor of C5 such as Nomacopan (Coversin; rVA576).

As used herein, the term “serum trough level” refers to the lowest level that the agent (e.g., the anti-C5 antibody, or antigen binding fragment thereof) or medicine is present in the serum. In contrast, a “peak serum level,” refers to the highest level of the agent in the serum. The “average serum level,” refers to the mean level of the agent in the serum over time.

### II. Alternative Pathway Inhibitors

The complement system is activated via three pathways (i.e., the classical pathway (CP), the lectin pathway (LP), and the alternative pathway (AP)) that converge to a common point, the activation of the C3 component (see, e.g., Ricklin D., et al., 2010., Nat. Immunol. 11: 785-797). The AP of complement activation is in a constant state of low-level activation (often referred to as “tickover”). C3 is hydrolyzed in the plasma to C3i, which has many of the properties of C3b. C3i then binds the plasma protein, Factor B. Bound Factor B is cleaved by Factor D to produce Ba and Bb. Ba is released and the remaining complex comprised of C3iBb forms the alternative pathway C3 convertase. Most of the C3b generated by the convertase is hydrolyzed. However, if C3b comes into contact with an invading micro-organism it binds and amplification of the alternative pathway is promoted by the binding of C3b to Factor B. The plasma protein, properdin, stabilizes the C3 convertase to prolong activity. C3b produced in this pathway also yields the C5 convertase, C3bBb3b, which leads to the production of C5a and C5b. Of note, C3b generated in the CP feeds into the AP to amplify the activation of complement.

Any suitable inhibitor of the AP of complement can be used in the methods described herein. In some embodiments, the inhibitor is one which inhibits a target upstream to complement 5 (C5). In some embodiments, the inhibitor is a C3 inhibitor. An exemplary C3 inhibitor is APL-2 (pegcetacoplan), a synthetic cyclic peptide conjugated to a polyethylene glycol (PEG) polymer that binds specifically to C3 and C3b. Representative inhibitors of complement pathways are provided in Table 1.

TABLE 1
Molecules and Targets Currently Being Evaluated for PNH Therapy

| Target | Therapeutic |
|---|---|
| MASP-3 | OMS906 (α-MASP-3 mAb) (Omeros) |
| Factor D | ACH-4471 (Achillion) |
| | Anti-FD mAb (Lampalizumab) (Genentech) |
| | BCX9930 (Biocryst) |
| | Various (see, Maibaum et al. (Nat Chem Biol. |
| | 2016 12(12): 1105-10)) (Novartis) |
| Factor B | LNPO23 (Novartis) |
| C3/C5 | Compstatin/Derivative - APL2, APL9 (Apellis) |
| Convertases | Compstatin/Derivative - AMY-101 (Amyndas) |
| | Mini-FH (Amyndas) |
| | CR2-Factor H/TT30 (Alexion) |
| C5 | Anti-C5 mAb eculizumab SOLIRIS ® (Alexion) |
| C5 - Follow | Eculizumab biosimilars, e.g., ABP 959 (Amgen); |
| up | Elizaria (Generium); SB 12 |
| | (Samsung)) |
| | Nomacopan (Coversin; rVA576) (Akari) |
| | Ravulizumab (ULTOMIRIS®) (Alexion) |
| | Tesidolumab (LFG3 16)(Novartis) |
| | Pozelimab (Regeneron) |
| | Crovalimab (SKY059)(Roche/Chugai) |

(Developer/Distributor names provided in parenthesis)

In some embodiments, the inhibitor is a complement factor D (CFD) inhibitor. Complement factor D is a serine proteinase with only a single known natural substrate: factor B bound to C3b (see, Volanakis, J. E., et al, 1993, Methods in Enzymol., 223:82-97). The serum concentration of factor D, 2 μg/ml, is the lowest of any complement protein (see, e.g., Liszewski, M. K. and J. P. Atkinson, 1993, In Fundamental Immunology, Third Edition. Edited by W. E. Paul. Raven Press, Ltd. New York). Factor D participates in C3 convertase generation by cleavage of factor B (FB) at two steps in the AP cascade: generation of the initial C3 convertase (C3(H2O)Bb) following spontaneous AP activation (tickover) in the fluid phase; and, the production of surface-bound C3 convertase (C3bBb) which mediates dramatic amplification of the initial activation (amplification loop) and activation of the terminal pathway, leading to opsonization of target surfaces by C3b, release of the anaphylatoxins C3a and C5a and formation of membrane attack complex (MAC) (see, e.g., Yuan et al., Haematologica. 2017 March; 102(3):466-475). Additional regulatory proteins can promote (properdin) or attenuate (factor H, factor I, multiple membrane-bound proteins including CD55 and CD59) AP activity.

As used herein, a “factor D inhibitor” or “CFD inhibitor” is a molecule or substance that prevents, reduces, or blocks the activity of Factor D. In some embodiments, the CFD inhibitor is an antibody, or an antigen-binding fragment thereof, e.g., an antibody, or an antigen-binding fragment thereof, that binds to Factor D. In some embodiments, the CFD inhibitor is a small molecule inhibitor. In some embodiments, the CFD inhibitor is a nucleotide (e.g., a DNA, an RNA, an shRNA, an miRNA, an siRNA, or an antisense DNA). In some embodiments, the CFD inhibitor is a peptide, a protein, a peptide mimetic, an aptamer, or any other molecule that binds to Factor D.

An exemplary CFD inhibitor is danicopan (also referred to as “ALXN2040”, “ACH-4471” and “ACH-0144471”). Danicopan is a selective and orally active small-molecule factor D inhibitor, which shows high binding affinity to human Factor D with a Kd value of 0.54 nM. Danicopan inhibits the AP of complement (APC) activity. In one embodiment, the CFD inhibitor comprises:

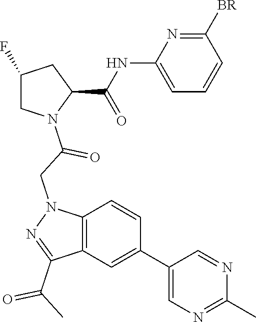

or a pharmaceutically acceptable salt thereof.

Additional exemplary CFD inhibitors include the small molecule CFD inhibitors taught by Maibaum, J., et al. (Nature Chemical Biology, volume 12, pages 1105-1110 (2016)), i.e., Compounds 1-7 or their pharmaceutically acceptable salts. Accordingly, in one embodiment, the CFD inhibitor comprises:

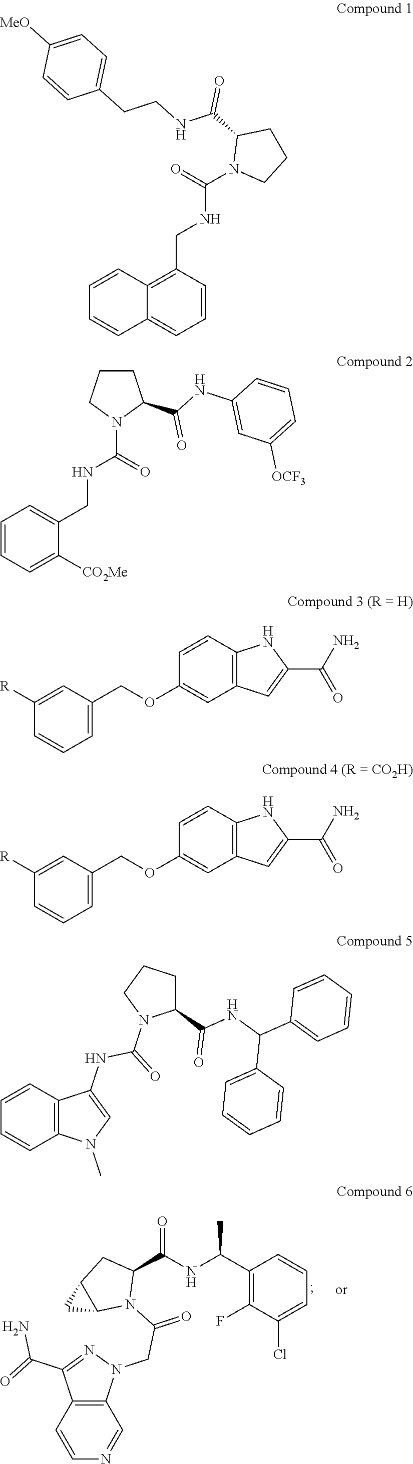

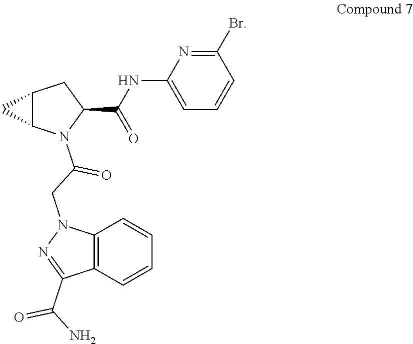 [SMILES: NC(=O)c1nn(CC(=O)N2[C@@H]3C[C@@H]3C[C@H]2C(=O)Nc2cccc(Br)n2)c2ccccc12]

Another exemplary CFD inhibitor is lampalizumab (also referred to as “FCFD4 14S” and “aFD”), an antigen-binding fragment of a humanized monoclonal antibody that binds to complement factor D. Specifically, lampalizumab is an antibody Fab fragment comprised of a 214-residue light chain (SEQ ID NO:51) and a 223 residue heavy chain (SEQ ID NO:52). Lampalizumab is described in WO2015168468 and U.S. Pat. No. 10,407,510, the teachings of which are expressly incorporated herein by reference. Additional exemplary CFD inhibitors include the anti-factor D antibodies described in US20190359699, including mAb 11-8A1, mAb 1F10-5, and variants thereof (see, e.g., paragraphs [007]-[0021]), the teachings of which are expressly incorporated herein by reference.

Other exemplary CFD inhibitors include the fused bicyclic ring compounds described in U.S. Pat. No. 6,653,340 (see, e.g., column 6 (line 15) through column 56 (line 48)), (including the CFD inhibitor BCX1470) and the compounds disclosed in Examples 1-20), as well as the particular CFD inhibitors described in US 20080269318, including BCX-1470 (see, e.g., paragraphs [0023] and [0032]), WO2012/093101 (see, e.g., pages 5-67), WO2014/002057 (see, e.g., pages 3-13), WO2014/009833 (see, e.g., pages 4-11), WO2014/002051 (see, e.g., pages 5-16), WO2014/002052 (see, e.g., pages 4-11), WO2014/002053 (see, e.g., pages 4-11), WO2014/002054 (see, e.g., pages 5-19), WO2014/002058 (see, e.g., pages 5-20), WO2014/002059 (see, e.g., pages 6-8 and pages 13-20), and WO2014/005150 (see, e.g., pages 3-4 and pages 7-30), the teachings and particular CFD inhibitors disclosed therein, which are all expressly incorporated herein by reference.

The CFD inhibitors described herein can be administered, for example, either systemically or locally. Systemic administration includes, for example, oral, transdermal, subdermal, intraperitioneal, subcutaneous, transnasal, sublingual, or rectal. Local administration includes, for example, topical administration. In one embodiment, the CFD inhibitor is danicopan administered orally.

In some embodiments, the inhibitor of the alternate complement component is a complement 3 (C3) inhibitor, which are useful for reducing effects of suboptimal C5 blockade, e.g., persistent anemia. It is postulated that an attenuation in the hematological benefit conferred by anti-C5 antibodies in many PNH patients may be due to opsonization of surviving PNH erythrocytes with C3 fragments, thereby reducing the in vivo half-life of erythrocytes. Therefore, in certain embodiments, the method of the present disclosure relates to use of C3 inhibitors (either alone or together with FD inhibitors) in the therapy of PNH patients who respond inadequately to C5 inhibitors, e.g., anti-C5 antibody therapy with eculizumab.

In some embodiments, the C3 inhibitor is a compstatin or an analog thereof. Compstatin is a cyclic peptide that binds to C3 and inhibits complement activation (see U.S. Pat. No. 6,319,897). Compstatin analogs that have higher complement inhibiting activity than compstatin have been developed, e.g., WO2004/026328 (see, e.g., U.S. Pat. No. 7,989,589). As used herein, the term “compstatin analog” includes complement inhibiting analog of compstatin. See, WO2017/062879; WO/2014/152391 and WO/2012/178083, the disclosures in these publications and U.S. counterparts thereof (see, e.g., US 2019-0381129; U.S. Pat. Nos. 10,308,687; and 10,039,802) are incorporated by reference herein in their entirety. Preferred compstatin analogs include pegcetacoplan (APL-2) and related molecules (e.g., APL-9). Compstatin analogs may be acetylated or amidated, e.g., at the N-terminus and/or C-terminus, specifically acetylated at the N-terminus and amidated at the C-terminus.

In some embodiments, the C3 inhibitor is a compstatin mimetic. Representative compstatin mimetics are provided in WO2004/026328; WO2007/062249; WO/2008/140637; WO/2015/142701, the disclosures in these publications and U.S. counterparts thereof (see, e.g., U.S. Pat. Nos. 7,989,589; 7,888,323; US 2011-0046075; and U.S. Pat. No. 10,213,476) are incorporated by reference herein in their entirety. Preferred compstatin mimetics include AMY-101 (CAS: 1427001-89-5).

In some embodiments, the alternate complement component modulates Factor H. Factor H regulates complement activation on cells and surfaces by possessing both cofactor activity for the Factor I mediated C3b cleavage, and decay accelerating activity against the alternative pathway C3-convertase, C3bBb. Factor H exerts its protective action on cells and self surfaces but not on the surfaces of bacteria or viruses. This is thought to be the result of Factor H having the ability to adopt conformations with lower or higher activities as a cofactor for C3 cleavage or decay accelerating activity. In preferred embodiments, the complement component is a mini factor H (mini-FH/AMY-201).

### III. Anti-C5 Antibodies

The anti-C5 antibodies described herein bind to complement component C5 (e.g., human C5) and inhibit the cleavage of C5 into fragments C5a and C5b. Anti-C5 antibodies (or VH/VL domains derived therefrom) suitable for use in the disclosure can be generated using methods well known in the art. Alternatively, art recognized anti-C5 antibodies can be used. Antibodies or any other agents that compete with any of these art-recognized antibodies for binding to C5 also can be used.

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered by a protein engineering technique (e.g., by mutation, deletion, substitution, conjugation to a non-antibody moiety). For example, an antibody may include one or more variant amino acids (compared to a naturally occurring antibody), which changes a property (e.g., a functional property) of the antibody. For example, numerous such alterations are known in the art which affect, e.g., half-life, effector function, and/or immune responses to the antibody in a patient. The term antibody also includes artificial or engineered polypeptide constructs which comprise at least one antibody-derived antigen binding site.

Eculizumab (also known as SOLIRIS®) is an anti-C5 antibody comprising heavy and light chains having sequences shown in SEQ ID NO: 10 and 11, respectively, or antigen binding fragments and variants thereof. The variable regions of SOLIRIS® are described in PCT/US1995/005688 and U.S. Pat. No. 6,355,245, the teachings of which are hereby incorporated by reference. The full heavy and light chains of SOLIRIS® are described in PCT/US2007/006606 (see, e.g., U.S. Pat. No. 9,718,880), the teachings of which are hereby incorporated by reference. In one embodiment the anti-C5 antibody, comprises the CDR1, CDR2, and CDR3 domains of the VH region of SOLIRIS® having the sequence set forth in SEQ ID NO: 7, and the CDR1, CDR2 and CDR3 domains of the VL region of SOLIRIS® having the sequence set forth in SEQ ID NO: 8. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises VH and VL regions having the amino acid sequences set forth in SEQ ID NO: 7 and SEQ ID NO: 8, respectively.

Another exemplary anti-C5 antibody is ULTOMIRIS® (ravulizumab) comprising heavy and light chains having the sequences shown in SEQ ID NOs:14 and 11, respectively, or antigen binding fragments and variants thereof. ULTOMIRIS® (ravulizumab) (also known as BNJ441 and ALXN1210) is described in PCT/US2015/019225 and U.S. Pat. No. 9,079,949, the teachings of which are hereby incorporated by reference. The terms ravulizumab, BNJ441, and ALXN1210 may be used interchangeably throughout this document, but all refer to the same antibody. ULTOMIRIS® (ravulizumab) selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ULTOMIRIS® (ravulizumab). For example, in one embodiment, the antibody comprises the CDR1, CDR2, and CDR3 domains of the VH region of ULTOMIRIS® (ravulizumab) having the sequence set forth in SEQ ID NO:12, and the CDR1, CDR2 and CDR3 domains of the VL region of ULTOMIRIS® (ravulizumab) having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region of the 8110 antibody having the sequence set forth in SEQ ID NO: 35, and the VL region of the 8110 antibody having the sequence set forth in SEQ ID NO: 36.

Another exemplary anti-C5 antibody is the 305LO5 antibody described in US2016/0176954A1. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region of the 305LO5 antibody having the sequence set forth in SEQ ID NO: 43, and the VL region of the 305LO5 antibody having the sequence set forth in SEQ ID NO: 44.

Another exemplary anti-C5 antibody is the SKY59 antibody described in Fukuzawa T., et al., Rep. 2017 Apr. 24; 7(1):1080). In one embodiment, the antibody comprises the heavy and light chain CDRs or variable regions of the SKY59 antibody. In another embodiment, the antibody, or antigen binding fragment thereof, comprises a heavy chain comprising SEQ ID NO: 45 and a light chain comprising SEQ ID NO: 46.

Another exemplary anti-C5 antibody is the REGN3918 antibody (also known as H4H12166PP) described in US20170355757 (see, U.S. Pat. No. 10,633,434). In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q311I, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434 of a native human IgG Fc constant region, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/4345 double substitution when used in a IgG2/4 chimeric Fc may correspond to 429L and 435S as in the M429L and N435S variants found in BNJ441 (ULTOMIRIS® (ravulizumab)) and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM.

In other embodiments, the [(K_{D} of the antibody for C5 at pH 6.0 at C)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500, or 8000).

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. (see the working examples). For example, the kinetics of antibody binding to human C5 can be determined at pH 8.0, 7.4, 7.0, 6.5 and 6.0 via surface plasmon resonance (SPR) on a BIAcore 3000 instrument using an anti-Fc capture method to immobilize the antibody.

In one embodiment, the anti-C5 antibody, or antigen binding fragment thereof, blocks the generation or activity of the C5a and/or C5b active fragments of a C5 protein (e.g., a human C5 protein). Through this blocking effect, the antibodies inhibit, e.g., the pro-inflammatory effects of C5a and the generation of the C5b-9 membrane attack complex (MAC) at the surface of a cell.

Methods for determining whether a particular antibody or therapeutic agent described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2nd Edition,” 135-240, Springfield, Ill., CC Thomas (1961), pages 135-139, or a conventional variation of that assay such as the chicken erythrocyte hemolysis method as described in, e.g., Hillmen et al. (2004) N Engl J Med 350(6):552. Methods for determining whether a candidate compound inhibits the cleavage of human C5 into forms C5a and C5b are known in the art and described in Evans et al. (1995) Mol Immunol 32(16):1183-95. For example, the concentration and/or physiologic activity of C5a and C5b in a body fluid can be measured by methods well known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of an anti-C5 antibody, or antigen binding fragment thereof, on complement activation. In order to determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50 eq assay. The CH50 eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50 eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55, or 60) % decrease in the activity of terminal complement in, e.g., a hemolytic assay or CH50 eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of SOLIRIS® (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90, or 95) % of the complement inhibitory activity of SOLIRIS® in a hemolytic assay or CH50 eq assay.

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

### IV. Compositions

The compositions can be formulated as a pharmaceutical solution, e.g., for administration to a subject for the treatment or prevention of a complement-associated disorder. The pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is an established art (see, for example, Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). In some embodiments, a composition can be formulated, for example, as a buffered solution at a suitable concentration and suitable for storage at 2-8 C (e.g., 4 C). In some embodiments, a composition can be formulated for storage at a temperature below OC (e.g., −20 C or −80 C). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1½ years or 2 years) at 2-8 C (e.g., 4 C). Thus, in some embodiments, the compositions described herein are stable in storage for at least 1 year at 2-8 C (e.g., 4 C).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

### V. Methods

Provided herein are methods for treating PNH in a subject who previously exhibited an inadequate response to an anti-C5 antibody therapy, by administering to the subject a therapeutically effective amount of an inhibitor of the AP. In some embodiments, the inhibitor of the AP is one which inhibits a target upstream to C5, such as Factor D or complement 3 (C3).

In some embodiments, the treatment results in a reduction in one or more of the following in the subject: (a) persistent extravascular hemolysis (EVH); (b) anemia; and/or (c) transfusion dependence; and/or an improvement in FACIT Fatigue Scale Score. In some embodiments, the control of MAC-mediated intravascular hemolysis in the inadequately responding PNH subject is maintained or improved following treatment.

In some embodiments, the inadequate response an anti-C5 antibody therapy is related to a pharmacokinetic (PK) aspect, for example, (a) ineffective inhibition of C5 cleavage in the subject; (b) low dose and/or low subject plasma levels of the anti-C5 antibody; (c) enhanced clearance of the anti-C5 antibody in the subject; and/or (d) anti-C5 antibody intolerance in the subject resulting in lowered anti-C5 antibody dosing, preferably wherein anti-C5 antibody intolerance comprises fatigue and post-infusion pain. In some embodiments, the inadequate response an anti-C5 antibody therapy is related to a pharmacodynamic (PD) aspect, for example, (a) CR1 polymorphism; (b) extra-vascular hemolysis (EVH), e.g., via opsonization of blood cells surviving intra-vascular hemolysis (IVH); and/or (c) impaired effect of anti-C5 antibody activity by C3 fragments. In some embodiments, the inadequate response an anti-C5 antibody therapy is related to one or more PK and PD aspects.

Also provided herein are methods for treating PNH in a human patient, comprising administering to the patient a CFD inhibitor alone or in combination with an anti-C5 antibody, or antigen binding fragment thereof. In some embodiments, the CFD inhibitor and/or anti-C5 antibody, or antigen binding fragment thereof, are administered (or are for administration) according to a particular clinical dosage regimen (e.g., at a particular dose amount and according to a specific dosing schedule).

In one embodiment, a method for treating PNH in a subject is provided, the method comprising: administering to the subject a therapeutically effective amount of a complement factor D (CFD) inhibitor in combination with a therapeutically effective amount of an anti-C5 antibody, or antigen binding fragment thereof, wherein the subject exhibits one or more of the following clinical improvements 12 and/or 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater compared to the subject's baseline FACIT Fatigue Scale Score.

In another embodiment, a method for treating PNH in a subject who previously exhibited an inadequate response to an anti-C5 antibody therapy is provided, the method comprising:

administering to the subject a therapeutically effective amount of a complement factor D (CFD) inhibitor,

wherein the inadequate response by the subject was transfusion dependence and/or anemia; and

wherein the subject exhibits one or more of the following clinical improvements 12 and/or 24 weeks post-treatment with the CFD inhibitor:
(d) hemoglobin increase of 2.0 g/dL or greater compared to the subject's baseline hemoglobin level;
(e) transfusion independence or transfusion avoidance; and/or
(f) FACIT Fatigue Scale Score increase of 10 points or greater compared to the subject's baseline FACIT Fatigue Scale Score.

In another embodiment, a method for treating PNH in a subject who previously exhibited an inadequate response to an anti-C5 antibody therapy is provided, the method comprising:

administering to the subject a therapeutically effective amount of a complement factor D (CFD) inhibitor in combination with a therapeutically effective amount of an anti-C5 antibody, or antigen binding fragment thereof,

wherein the inadequate response by the subject was transfusion dependence and/or anemia; and

wherein the subject exhibits one or more of the following clinical improvements 12 and/or 24 weeks post-treatment with the CFD inhibitor:
(a) hemoglobin increase of 2.0 g/dL or greater compared to the subject's baseline hemoglobin level;
(b) transfusion independence or transfusion avoidance; and/or
(c) FACIT Fatigue Scale Score increase of 10 points or greater compared to the subject's baseline FACIT Fatigue Scale Score.

In some embodiments, the methods further comprise determining the subject's hemoglobin level, transfusion status, and/or FACIT Fatigue Scale Score at baseline and 12 and/or 24 weeks post-treatment, wherein (a) a hemoglobin increase of 2.0 g/dL or greater compared to the subject's baseline hemoglobin level; (b) transfusion independence or transfusion avoidance; and/or (c) a FACIT Fatigue Scale Score increase of 10 points or greater compared to the subject's baseline FACIT Fatigue Scale Score is indicative of treatment. In some embodiments, the methods involve treating a subject having PNH who previously exhibited an inadequate response to an anti-C5 antibody therapy (e.g., SOLIRIS®, ULTOMIRIS®, 7086 antibody, 8110 antibody, 305LO5 antibody, SKY59 antibody, or REGN3918 antibody). In some embodiments, the subject having PNH previously exhibited an inadequate response to SOLIRIS®. In some embodiments, the subject having PNH previously exhibited an inadequate response to SOLIRIS® at an approved dose or higher for ≥24 weeks (e.g., 24, 25, 26, 27, 28, 29, 30, 31, 32, 33, 34, 35, 36, 37, 38, 39, 40 or more weeks) without change in regimen ≤8 weeks. In some embodiments, the subject having PNH previously exhibited an inadequate response to ULTOMIRIS®.

In some embodiments, the inadequate response by the subject was transfusion dependence (e.g., ≥1 red blood cell (RBC) transfusion ≤12 weeks prior to screening). In some embodiments, the inadequate response by the subject was anemia (e.g., hemoglobin <10 g/dl). In some embodiments, the inadequate response by the subject was transfusion dependence and anemia.

In some embodiments, the method further comprises determining the subject's hemoglobin level, transfusion status, and/or FACIT Fatigue Scale Score at baseline and 12 and/or 24 weeks post-treatment, wherein
(a) a hemoglobin increase of 2.0 g/dL or greater compared to the subject's baseline hemoglobin level;
(b) transfusion independence or transfusion avoidance; and/or
(c) a FACIT Fatigue Scale Score increase of 10 points or greater compared to the subject's baseline FACIT Fatigue Scale Score, is indicative of treatment.

In some embodiments, the CFD inhibitor (e.g., danicopan) is administered (or is for administration) according to a particular clinical dosage regimen (e.g., at a particular dose amount and according to a specific dosing schedule). In some embodiments, the CFD inhibitor is administered orally to the subject. In some embodiments, the CFD inhibitor is administered orally three times daily (TID) to the subject. In some embodiments, the CFD inhibitor is administered orally at a dose of between about 50 mg to 300 mg to the subject. In some embodiments, the CFD inhibitor is administered orally at a dose of about 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg. In some embodiments, the CFD inhibitor is administered orally at a dose of about 100 mg. In some embodiments, the CFD inhibitor is administered orally at a dose of about 100 mg TID. In some embodiments, the CFD inhibitor is administered orally at a dose of about 150 mg. In some embodiments, the CFD inhibitor is administered orally at a dose of about 150 mg TID. In some embodiments, the CFD inhibitor is administered orally at a dose of about 200 mg. In some embodiments, the CFD inhibitor is administered orally at a dose of about 200 mg TID.

In some embodiments, the CFD inhibitor is administered for 4 weeks or more (e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks or more). In some embodiments, the CFD inhibitor is administered for 24 weeks. In some embodiments, the CFD inhibitor is administered for 9 months, 12 months, 15 months, 20 months, 24 months or longer. In some embodiments, the CFD inhibitor is administered for 1, 2, 3, 4, 5, 6 or more years.

In some embodiments, the anti-C5 antibody, or antigen binding fragment thereof (e.g., SOLIRIS® or ULTOMIRIS®), is administered (or is for administration) according to a particular clinical dosage regimen (e.g., at a particular dose amount and according to a specific dosing schedule). The anti-C5 antibodies, or antigen binding fragments thereof, can be administered to a patient by any suitable means. In some embodiments, the anti-C5 antibody, or antigen binding fragment thereof, is administered intravenously to the subject.

In some embodiments, the dose of the anti-C5 antibody, or antigen binding fragment thereof, is a fixed dose. For example, in some embodiments, the anti-C5 antibody, or antigen binding fragment thereof, is administered to the subject at a dose of 600 mg weekly. In some embodiments, the anti-C5 antibody, or antigen binding fragment thereof, is administered to the subject at a dose of 900 mg every two weeks.

In some embodiments, the anti-C5 antibody, or antigen binding fragment thereof, is administered to the subject at a dose of 600 mg weekly for four doses, followed by a dose of 900 mg at Week 5 and then at a dose of 900 mg every 2 weeks thereafter. In some embodiments, SOLIRIS® is administered to the subject (e.g., an adult subject) at a dose of 600 mg weekly for four doses, followed by a dose of 900 mg at Week 5 and then at a dose of 900 mg every 2 weeks thereafter.

In some embodiments, the anti-C5 antibody, or antigen binding fragment thereof, is administered to a subject less than 18 years of age at a dose of 900 mg weekly for four doses to a subject weighing 40 kg and over, followed by a dose of 1200 mg at Week 5 and then at a dose of 1200 mg every two weeks thereafter. In some embodiments, SOLIRIS® is administered to a subject less than 18 years of age at a dose of 900 mg weekly for four doses to a subject weighing 40 kg and over, followed by a dose of 1200 mg at Week 5 and then at a dose of 1200 mg every two weeks thereafter.

In some embodiments, the anti-C5 antibody, or antigen binding fragment thereof, is administered to a subject less than 18 years of age at a dose of 600 mg weekly for two doses to a subject weighing 30 kg to less than 40 kg, followed by a dose of 900 mg at Week 3 and then at a dose of 900 mg every two weeks thereafter. In some embodiments, SOLIRIS® is administered to a subject less than 18 years of age at a dose of 600 mg weekly for two doses to a subject weighing 30 kg to less than 40 kg, followed by a dose of 900 mg at Week 3 and then at a dose of 900 mg every two weeks thereafter.

In some embodiments, the anti-C5 antibody, or antigen binding fragment thereof, is administered to a subject less than 18 years of age at a dose of 600 mg weekly for two doses to a subject weighing 20 kg to less than 30 kg, followed by a dose of 600 mg at Week 3 and then at a dose of 600 mg every two weeks thereafter. In some embodiments, SOLIRIS® is administered to a subject less than 18 years of age at a dose of 600 mg weekly for two doses to a subject weighing 20 kg to less than 30 kg, followed by a dose of 600 mg at Week 3 and then at a dose of 600 mg every two weeks thereafter.

In some embodiments, the anti-C5 antibody, or antigen binding fragment thereof, is administered to a subject less than 18 years of age at a dose of 600 mg weekly for one dose to a subject weighing 10 kg to less than 20 kg, followed by a dose of 300 mg at Week 3 and then at a dose of 300 mg every two weeks thereafter. In some embodiments, SOLIRIS® is administered to a subject less than 18 years of age at a dose of 600 mg weekly for one dose to a subject weighing 10 kg to less than 20 kg, followed by a dose of 300 mg at Week 3 and then at a dose of 300 mg every two weeks thereafter.

In some embodiments, the anti-C5 antibody, or antigen binding fragment thereof, is administered to a subject less than 18 years of age at a dose of 300 mg weekly for one dose to a subject weighing 5 kg to less than 10 kg, followed by a dose of 300 mg at Week 2 and then at a dose of 300 mg every three weeks thereafter. In some embodiments, SOLIRIS® is administered to a subject less than 18 years of age at a dose of 300 mg weekly for one dose to a subject weighing 5 kg to less than 10 kg, followed by a dose of 300 mg at Week 2 and then at a dose of 300 mg every three weeks thereafter.

In some embodiments, the dose of the anti-C5 antibody, or antigen binding fragment thereof, is based on the weight of the patient. In some embodiments, for example, 300 mg of the anti-C5 antibody, or antigen binding fragment thereof, is administered to a patient weighing ≥5 to <10 kg. In some embodiments, 600 mg of the anti-C5 antibody, or antigen binding fragment thereof, is administered to a patient weighing ≥10 to <20 kg. In some embodiments, 900 mg or 2100 mg of the anti-C5 antibody, or antigen binding fragment thereof, is administered to a patient weighing ≥20 to <30 kg. In some embodiments, 1200 mg or 2700 mg of the anti-C5 antibody, or antigen binding fragment thereof, is administered to a patient weighing ≥30 to <40 kg. In some embodiments, 2400 mg or 3000 mg of the anti-C5 antibody, or antigen binding fragment thereof, is administered to a patient weighing ≥40 to <60 kg. In some embodiments, 2700 mg or 3300 mg of the anti-C5 antibody, or antigen binding fragment thereof, is administered to a patient weighing ≥60 to <100 kg. In some embodiments, 3000 mg or 3600 mg of the anti-C5 antibody, or antigen binding fragment thereof, is administered to a patient weighing ≥100 kg. In certain embodiments, dosage regimens are adjusted to provide the optimum desired response (e.g., an effective response).

In some embodiments, the anti-C5 antibody, or antigen binding fragment thereof, is administered:
(a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and
(b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In some embodiments, ULTOMIRIS®, is administered:
(a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and
(b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In some embodiments, the anti-C5 antibody, or antigen binding fragment thereof, is administered to a patient weighing ≥40 to <60 kg:
(a) once on Day 1 of the administration cycle at a dose of 2400 mg; and
(b) on Day 15 of the administration cycle and every eight weeks thereafter at a dose of 3000 mg.

In some embodiments, ULTOMIRIS® is administered to a patient weighing ≥40 to <60 kg:
(a) once on Day 1 of the administration cycle at a dose of 2400 mg; and
(b) on Day 15 of the administration cycle and every eight weeks thereafter at a dose of 3000 mg.

In some embodiments, the anti-C5 antibody, or antigen binding fragment thereof, is administered to a patient weighing ≥60 to <100 kg:
(a) once on Day 1 of the administration cycle at a dose of 2700 mg; and
(b) on Day 15 of the administration cycle and every eight weeks thereafter at a dose of 3300 mg.

In some embodiments, ULTOMIRIS® is administered to a patient weighing ≥60 to <100 kg:
(a) once on Day 1 of the administration cycle at a dose of 2700 mg; and
(b) on Day 15 of the administration cycle and every eight weeks thereafter at a dose of 3300 mg.

In some embodiments, the anti-C5 antibody, or antigen binding fragment thereof, is administered to a patient weighing ≥100 kg:
(a) once on Day 1 of the administration cycle at a dose of 3000 mg; and
(b) on Day 15 of the administration cycle and every eight weeks thereafter at a dose of 3600 mg.

In some embodiments, ULTOMIRIS® is administered to a patient weighing ≥100 kg:
(a) once on Day 1 of the administration cycle at a dose of 3000 mg; and
(b) on Day 15 of the administration cycle and every eight weeks thereafter at a dose of 3600 mg.

In some embodiments, the anti-C5 antibody, or antigen binding fragment thereof, is administered to a subject less than 18 years of age:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and
(b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In some embodiments, the anti-C5 antibody, or antigen binding fragment thereof, is administered to a patient weighing ≥5 to <10 kg: (a) once on Day 1 at a dose of 600 mg; and (b) on Day 15 and every four weeks thereafter at a dose of 300 mg. In some embodiments, ULTOMIRIS® is administered to a patient weighing ≥5 to <10 kg: (a) once on Day 1 at a dose of 600 mg; and (b) on Day 15 and every four weeks thereafter at a dose of 300 mg.

In some embodiments, the anti-C5 antibody, or antigen binding fragment thereof, is administered to a patient weighing ≥10 to <20 kg: (a) once on Day 1 at a dose of 600 mg; and (b) on Day 15 and every four weeks thereafter at a dose of 600 mg. In some embodiments, the anti-C5 antibody is administered to a patient weighing ≥10 to <20 kg: (a) once on Day 1 at a dose of 600 mg; and (b) on Day 15 and every four weeks thereafter at a dose of 600 mg.

In some embodiments, the anti-C5 antibody, or antigen binding fragment thereof, is administered to a patient weighing ≥20 to <30 kg: (a) once on Day 1 at a dose of 900 mg; and (b) on Day 15 and every eight weeks thereafter at a dose of 2100 mg. In some embodiments, ULTOMIRIS® is administered to a patient weighing ≥20 to <30 kg: (a) once on Day 1 at a dose of 900 mg; and (b) on Day 15 and every eight weeks thereafter at a dose of 2100 mg.

In some embodiments, the anti-C5 antibody, or antigen binding fragment thereof, is administered to a patient weighing ≥30 to <40 kg: (a) once on Day 1 at a dose of 1200 mg; and (b) on Day 15 and every eight weeks thereafter at a dose of 2700 mg. In some embodiments, ULTOMIRIS® is administered to a patient weighing ≥30 to <40 kg: (a) once on Day 1 at a dose of 1200 mg; and (b) on Day 15 and every eight weeks thereafter at a dose of 2700 mg.

In some embodiments, the anti-C5 antibody, or antigen binding fragment thereof, is administered to a patient weighing ≥40 to <60 kg: (a) once on Day 1 at a dose of 2400 mg; and (b) on Day 15 and every eight weeks thereafter at a dose of 3000 mg. In some embodiments, ULTOMIRIS® is administered to a patient weighing ≥40 to <60 kg: (a) once on Day 1 at a dose of 2400 mg; and (b) on Day 15 and every eight weeks thereafter at a dose of 3000 mg.

In some embodiments, the anti-C5 antibody, or antigen binding fragment thereof, is administered to a patient weighing ≥60 to <100 kg: (a) once on Day 1 at a dose of 2700 mg; and (b) on Day 15 and every eight weeks thereafter at a dose of 3300 mg. In some embodiments, ULTOMIRIS® is administered to a patient weighing ≥60 to <100 kg: (a) once on Day 1 at a dose of 2700 mg; and (b) on Day 15 and every eight weeks thereafter at a dose of 3300 mg.

In some embodiments, the anti-C5 antibody, or antigen binding fragment thereof, is administered to a patient weighing ≥100 kg: (a) once on Day 1 at a dose of 3000 mg; and (b) on Day 15 and every eight weeks thereafter at a dose of 3600 mg. In some embodiments, ULTOMIRIS® is administered to a patient weighing ≥100 kg: (a) once on Day 1 at a dose of 3000 mg; and (b) on Day 15 and every eight weeks thereafter at a dose of 3600 mg.

In another aspect, the treatment regimens described are sufficient to maintain particular serum trough concentrations of the anti-C5 antibody, or antigen binding fragment thereof. In one embodiment, for example, the treatment regimen maintains a serum trough concentration of the anti-C5 antibody, or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In some embodiments, the treatment regimen maintains a serum trough concentration of the anti-C5 antibody, or antigen binding fragment thereof of 100 μg/mL or greater, 150 μg/mL or greater, 200 μg/mL or greater, 250 μg/mL or greater, or 300 μg/mL or greater. In some embodiments, the treatment maintains a serum trough concentration of the anti-C5 antibody, or antigen binding fragment thereof of between 100 μg/mL and 200 μg/mL. In some embodiments, the treatment maintains a serum trough concentration of the anti-C5 antibody, or antigen binding fragment thereof of about 175 μg/mL.

In some embodiments, to obtain an effective response, the anti-C5 antibody, or antigen binding fragment thereof, is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg or 260 μg of antibody per milliliter of the patient's blood. In some embodiments, the anti-C5 antibody is administered to the patient in an amount and with a frequency to maintain between 50 μg and 250 μg of antibody per milliliter of the patient's blood. In some embodiments, the anti-C5 antibody is administered to the patient in an amount and with a frequency to maintain between 100 μg and 200 μg of antibody per milliliter of the patient's blood. In some embodiments, the anti-C5 antibody is administered to the patient in an amount and with a frequency to maintain about 175 μg of antibody per milliliter of the patient's blood.

In one embodiment, a method for PNH in a subject who had an inadequate response to prior treatment with SOLIRIS® (eculizumab) is provided, the method comprising:

administering to the subject a therapeutically effective amount of danicopan in combination with a therapeutically effective amount of SOLIRIS® (eculizumab),

wherein the inadequate response by the subject was transfusion dependence and/or anemia; and

wherein danicopan is administered to the subject orally at a dose of 100 mg, 150 mg, or 200 mg TID to the subject;

wherein SOLIRIS® (eculizumab) is administered intravenously to the subject at a dose of 600 mg weekly for four doses, followed by a dose of 900 mg at Week 5 and then at a dose of 900 mg every 2 weeks thereafter; and

wherein the subject exhibits one or more of the following clinical improvements 12 and/or 24 weeks post-treatment with the CFD inhibitor:
(a) hemoglobin increase of 2.0 g/dL or greater compared to the subject's baseline hemoglobin level;
(b) transfusion independence or transfusion avoidance; and/or
(c) FACIT Fatigue Scale Score increase of 10 points or greater compared to the subject's baseline FACIT Fatigue Scale Score.

In another embodiment, a method for treating PNH in a subject who had an inadequate response to prior treatment with SOLIRIS® (eculizumab) is provided, the method comprising: administering to the subject a therapeutically effective amount of danicopan in combination with a therapeutically effective amount of SOLIRIS® (eculizumab),

wherein the inadequate response by the subject was transfusion dependence and/or anemia; and

wherein danicopan is administered to the subject orally at a dose of 100 mg, 150 mg, or 200 mg TID to the subject;

wherein SOLIRIS® (eculizumab) is administered intravenously to a subject less than 18 years of age:
(a) at a dose of 900 mg weekly for four doses to a subject weighing 40 kg and over, followed by a dose of 1200 mg at Week 5 and then at a dose of 1200 mg every two weeks thereafter;
(b) at a dose of 600 mg weekly for two doses to a subject weighing 30 kg to less than 40 kg, followed by a dose of 900 mg at Week 3 and then at a dose of 900 mg every two weeks thereafter;
(c) at a dose of 600 mg weekly for two doses to a subject weighing 20 kg to less than 30 kg, followed by a dose of 600 mg at Week 3 and then at a dose of 600 mg every two weeks thereafter;
(d) at a dose of 600 mg weekly for one dose to a subject weighing 10 kg to less than 20 kg, followed by a dose of 300 mg at Week 3 and then at a dose of 300 mg every two weeks thereafter; or
(e) at a dose of 300 mg weekly for one dose to a subject weighing 5 kg to less than 10 kg, followed by a dose of 300 mg at Week 2 and then at a dose of 300 mg every three weeks thereafter; and

wherein the subject exhibits one or more of the following clinical improvements 12 and/or 24 weeks post-treatment with the CFD inhibitor:
iv. hemoglobin increase of 2.0 g/dL or greater compared to the subject's baseline hemoglobin level;
v. transfusion independence or transfusion avoidance; and/or
vi. FACIT Fatigue Scale Score increase of 10 points or greater compared to the subject's baseline FACIT Fatigue Scale Score.

In another embodiment, a method for treating PNH in a subject is provided, the method comprising:

administering to the subject a therapeutically effective amount of danicopan in combination with a therapeutically effective amount of SOLIRIS® (eculizumab),

wherein danicopan is administered to the subject orally at a dose of 100 mg, 150 mg, or 200 mg TID to the subject;
wherein SOLIRIS® (eculizumab) is administered intravenously to the subject at a dose of 600 mg weekly for four doses, followed by a dose of 900 mg at Week 5 and then at a dose of 900 mg every 2 weeks thereafter; and
wherein the subject exhibits one or more of the following clinical improvements 12 and/or 24 weeks post-treatment with the CFD inhibitor;
(a) hemoglobin increase of 2.0 g/dL or greater compared to the subject's baseline hemoglobin level;
(b) transfusion independence or transfusion avoidance; and/or
(c) FACIT Fatigue Scale Score increase of 10 points or greater compared to the subject's baseline FACIT Fatigue Scale Score.

In another embodiment, a method for treating PNH in a subject less than 18 years of age is provided, the method comprising administering to the subject a therapeutically effective amount of danicopan in combination with a therapeutically effective amount of SOLIRIS® (eculizumab),

wherein danicopan is administered to the subject orally at a dose of 100 mg, 150 mg, or 200 mg TID to the subject;

wherein SOLIRIS® (eculizumab) is administered intravenously:
(a) at a dose of 900 mg weekly for four doses to a subject weighing 40 kg and over, followed by a dose of 1200 mg at Week 5 and then at a dose of 1200 mg every two weeks thereafter;
(b) at a dose of 600 mg weekly for two doses to a subject weighing 30 kg to less than 40 kg, followed by a dose of 900 mg at Week 3 and then at a dose of 900 mg every two weeks thereafter;
(c) at a dose of 600 mg weekly for two doses to a subject weighing 20 kg to less than 30 kg, followed by a dose of 600 mg at Week 3 and then at a dose of 600 mg every two weeks thereafter;
(d) at a dose of 600 mg weekly for one dose to a subject weighing 10 kg to less than 20 kg, followed by a dose of 300 mg at Week 3 and then at a dose of 300 mg every two weeks thereafter; or
(e) at a dose of 300 mg weekly for one dose to a subject weighing 5 kg to less than 10 kg, followed by a dose of 300 mg at Week 2 and then at a dose of 300 mg every three weeks thereafter; and
wherein the subject exhibits one or more of the following clinical improvements 12 and/or 24 weeks post-treatment with the CFD inhibitor:
i. hemoglobin increase of 2.0 g/dL or greater compared to the subject's baseline hemoglobin level;
ii. transfusion independence or transfusion avoidance; and/or
iii. FACIT Fatigue Scale Score increase of 10 points or greater compared to the subject's baseline FACIT Fatigue Scale Score.

In some embodiments, the methods described herein further comprise determining the subject's hemoglobin level, transfusion status, and/or FACIT Fatigue Scale Score at baseline and 12 and/or 24 weeks post-treatment, wherein (a) a hemoglobin increase of 2.0 g/dL or greater compared to the subject's baseline hemoglobin level; (b) transfusion independence or transfusion avoidance; and/or (c) a FACIT Fatigue Scale Score increase of 10 points or greater compared to the subject's baseline FACIT Fatigue Scale Score, is indicative of treatment.

### VI. Outcomes

Provided herein are methods for treating PNH in a patient. Symptoms of PNH include, but are not limited to, pallor, fatigue (e.g., tiredness, difficultly performing daily activities, trouble concentrating, dizziness, weakness), pain (e.g., stomach pain, leg pain or swelling, chest pain, back pain), dark-colored urine, shortness of breath, difficulty swallowing, yellowing of the skin and/or eyes, anemia, cytopenia, erectile dysfunction, blood clots, kidney disease, damage to organs, stroke or heart attack.

Patients treated according to the methods disclosed herein experience improvement in at least one sign of PNH. The treatment may produce at least one therapeutic effect selected from the group consisting of, for example, a reduction or cessation in pallor, fatigue, jaundice, anemia, cytopenia, abdominal pain, dyspnea, dysphagia, chest pain or erectile dysfunction.

In some embodiments, the subject exhibits one or more other clinical improvements after being treated according to the methods described herein. For example, in one embodiment, the subject exhibits a hemoglobin increase of 2.0 g/dL or greater after treatment compared to the subject's baseline hemoglobin level. In some embodiments, the subject exhibits a hemoglobin increase of 2.0 g/dL or greater after 12, 13, 14, 15, 16, 17, 18, 19, 20, 21, 22, 23, 24, 25, 26, 27, 28, 29, or 30 weeks of treatment compared to the subject's baseline hemoglobin level. In some embodiments, the subject exhibits a hemoglobin increase of 2.0 g/dL or greater after 24 weeks of treatment compared to the subject's baseline hemoglobin level.

In some embodiments, the subject exhibits transfusion independence after treatment. In some embodiments, the subject exhibits transfusion independence after 12, 13, 14, 15, 16, 17, 18, 19, 20, 21, 22, 23, 24, 25, 26, 27, 28, 29, or 30 weeks of treatment. In some embodiments, the subject exhibits transfusion independence after 24 weeks of treatment. In some embodiments, the subject exhibits transfusion avoidance after treatment. In some embodiments, the subject exhibits transfusion avoidance after 12 weeks of treatment.

In some embodiments, the subject exhibits a FACIT Fatigue Scale Score increase of 10 points or greater, e.g., 10, 11, 12, after treatment compared to the subject's baseline FACIT Fatigue Scale Score. In some embodiments, the subject exhibits a FACIT Fatigue Scale Score increase of 10 points or greater after 12, 13, 14, 15, 16, 17, 18, 19, 20, 21, 22, 23, 24, 25, 26, 27, 28, 29, or 30 weeks of treatment. In some embodiments, the subject exhibits a FACIT Fatigue Scale Score increase of 10 points or greater after 12 and/or 24 weeks of treatment.

In some embodiments, the subject exhibits a hemoglobin increase of 2.0 g/dL compared to the subject's baseline hemoglobin level and transfusion independence, after treatment (e.g., after 12, 13, 14, 15, 16, 17, 18, 19, 20, 21, 22, 23, 24, 25, 26, 27, 28, 29, or 30 weeks of treatment). In some embodiments, the subject exhibits a hemoglobin increase of 2.0 g/dL compared to the subject's baseline hemoglobin level and transfusion independence, after 12 and/or 24 weeks treatment.

In some embodiments, the subject exhibits a hemoglobin increase of 2.0 g/dL compared to the subject's baseline hemoglobin level and a FACIT Fatigue Scale Score increase of 10 points or greater, after treatment (e.g., after 12, 13, 14, 15, 16, 17, 18, 19, 20, 21, 22, 23, 24, 25, 26, 27, 28, 29, or 30 weeks of treatment). In some embodiments, the subject exhibits a hemoglobin increase of 2.0 g/dL compared to the subject's baseline hemoglobin level and a FACIT Fatigue Scale Score increase of 10 points or greater, after 12 and/or 24 weeks treatment.

In some embodiments, the subject exhibits transfusion independence or transfusion avoidance and a FACIT Fatigue Scale Score increase of 10 points or greater, after treatment (e.g., after 12, 13, 14, 15, 16, 17, 18, 19, 20, 21, 22, 23, 24, 25, 26, 27, 28, 29, or 30 weeks of treatment). In some embodiments, the subject exhibits transfusion independence or transfusion avoidance and a FACIT Fatigue Scale Score increase of 10 points or greater, after 12 or 24 weeks treatment

In some embodiments, the subject exhibits a hemoglobin increase of 2.0 g/dL compared to the subject's baseline hemoglobin level, transfusion independence or transfusion avoidance, and a FACIT Fatigue Scale Score increase of 10 points or greater, after treatment (e.g., after 12, 13, 14, 15, 16, 17, 18, 19, 20, 21, 22, 23, 24, 25, 26, 27, 28, 29, or 30 weeks of treatment). In some embodiments, the subject exhibits a hemoglobin increase of 2.0 g/dL compared to the subject's baseline hemoglobin level, transfusion independence or transfusion avoidance, and a FACIT Fatigue Scale Score increase of 10 points or greater, after 12 or 24 weeks treatment.

In some embodiments, the treatment results in a shift toward normal levels of bilirubin (e.g., from about 0.2-1.2 mg/dL).

In some embodiments, the treatment results in a reduction in reticulocytes compared to baseline (e.g., a 2, 3, 4 or 5-fold reduction).

In some embodiment, the treatment results in an increase in PNH specific red blood cell clone size compared to baseline (e.g., a 2, 3, 4, or 5-fold increase).

In some embodiments, the treatment results in a decrease in PNH erythrocytes opsonized with C3 fragment compared to baseline (e.g., a 2, 3, 4, or 5-fold reduction).

In some embodiments, the treatment produces a reduction in the need for blood transfusions compared to baseline.

In some embodiments, the treatment produces transfusion avoidance.

In some embodiments, the treatment results in terminal complement inhibition.

In some embodiments, the treatment produces a shift toward normal levels of at least one or more hemolysis-related hematologic biomarkers selected from the group consisting of: free hemoglobin, haptoglobin, reticulocyte count, PNH red blood cell (RBC) clone and/or D-dimer.

In some embodiments, lactate dehydrogenase (LDH) levels can be used to evaluate responsiveness to a therapy (e.g., a reduction of hemolysis as assessed by lactate dehydrogenase (LDH) levels is indicative of an improvement in at least one sign of PNH). LDH is a marker of intravascular hemolysis (Hill, A. et al., Br. J. Haematol., 149:414-25, 2010; Hillmen, P. et al., N. Engl. J. Med., 350:552-9, 2004; Parker, C. et al., Blood, 106:3699-709, 2005). Red blood cells contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):359 Abs.0903, 2008; Kanakura, Y. et al., Int. J. Hematol., 93:36-46, 2011). LDH concentration obtained at baseline and then serially throughout a treatment period, is an important measure of hemolysis. Baseline levels of cell-free plasma hemoglobin are highly elevated in patients with PNH with LDH≥1.5-fold above the upper limit of normal (LDH≥1.5×ULN), with a significant correlation between LDH and cell-free plasma hemoglobin (Hillmen, P. et al., N. Engl. J. Med., 355:1233-43, 2006). The normal LDH value range is 105-333 IU/L (international units per liter).

LDH levels can be measured using any suitable test or assay, such as those described by Ferri F F, ed. Ferri's Clinical Advisor 2014. Philadelphia: Pa: Elsevier Mosby; 2014: Section IV—Laboratory tests and interpretation of results. LDH concentration can be measured in various samples obtained from a patient, in particular, serum samples. As used herein, the term “sample” refers to biological material from a subject. Although serum LDH concentration is of interest, samples can be derived from other sources, including, for example, single cells, multiple cells, tissues, tumors, biological fluids, biological molecules or supernatants or extracts of any of the foregoing. Examples include tissue removed for biopsy, tissue removed during resection, blood, urine, lymph tissue, lymph fluid, cerebrospinal fluid, mucous and stool samples. The sample used can vary based on the assay format, the detection method and the nature of the tumors, tissues, cells or extracts to be assayed. Methods for preparing samples are known in the art and can be readily adapted to obtain a sample that is compatible with the method utilized.

In some embodiments, patients treated according to the disclosed methods experience reductions in LDH levels to near normal levels or to within 10%, 20%, 30%, 40% or within 50% below what is considered the normal level (e.g., within 105-333 IU/L (international units per liter). In some embodiments, the patient's LDH levels are normalized throughout maintenance period of treatment. In some embodiments, the treated patient's LDH levels are normalized at least at least 95% of the time while on the maintenance period of treatment. In some embodiments, the treated patient's LDH levels are normalized at least at least 90%, 85% or 80% of the time while on the maintenance period of treatment. In some embodiments, the patient's LDH levels are ≥1.5 fold above the upper limit of normal (LDH≥1.5×ULN) prior to initiating treatment.

In some embodiments, the treatment produces a reduction in major adverse vascular events (MAVEs; e.g., thrombophlebitis/deep vein thrombosis, pulmonary embolus, myocardial infarction, transient ischemic attack, unstable angina, renal vein thrombosis/renal artery thrombosis/glomerular thrombosis, renal infarction, acute peripheral vascular occlusion, mesenteric/visceral vein/arterial thrombosis or infarction, hepatic/portal vein thrombosis, cerebral arterial occlusion/cerebrovascular accident, cerebral venous occlusion, renal arterial thrombosis, or multi-infarct dementia).

In some embodiments, the treatment produces a shift toward normal levels of a chronic disease associated biomarker selected from the group consisting estimated glomerular filtration rate (eGFR) and spot urine:albumin:creatinine and plasma brain natriuretic peptide (BNP).

In some embodiments, the treatment produces a change from baseline in quality of life, assessed via version 4 and the European Organisation for Research and Treatment of Cancer, Quality of Life Questionnaire-Core 30 Scale compared to baseline.

### VII. Kits

Also provided herein are kits for treating PNH. The kits optionally also can include instructions, e.g., comprising administration schedules, to allow a practitioner (e.g., a physician, nurse, or patient) to administer the composition(s) contained therein to administer the composition(s) to a patient having PNH. The kit also can include a syringe.

Optionally, the kits include multiple packages of the single-dose pharmaceutical composition(s) each containing an effective amount of the CFD inhibitor and/or anti-C5 antibody for administration in accordance with the methods provided above. Instruments or devices necessary for administering the pharmaceutical composition(s) also may be included in the kits. For instance, a kit may provide one or more pre-filled syringes containing an effective amount of the CFD inhibitor and/or anti-C5 antibody.

In some embodiments, the kit comprises: (a) a dose of a complement factor D (CFD) inhibitor and (b) instructions for using the CFD, in any of the methods described herein. In some embodiments, the kit comprises: (a) a dose of a complement factor D (CFD) inhibitor, (b) a dose of an anti-C5 antibody; and (c) instructions for using the CFD and anti-C5 antibody, in any of the methods described herein. In some embodiments, the CFD is danicopan. In some embodiments, the anti-C5 antibody is eculizumab (e.g., SOLIRIS®) or ravulizumab (e.g., ULTOMIRIS®).

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### EXAMPLES

### Example 1: A Phase 2 Open-Label Study of Danicopan (ACH 4471) in Patients with Paroxysmal Nocturnal Hemoglobinuria (PNH) Who have an Inadequate Response to SOLIRIS® Monotherapy

A phase 2 clinical study was conducted in PNH patients who have had an inadequate response to SOURIS® (eculizumab) monotherapy from June 2018 to September 2019. Specifically, the aim of the study was to evaluate the addition of the factor D inhibitor, danicopan, on transfusion requirements in PNH patients with a suboptimal response to SOURIS® (eculizumab).

A. Methods

1. Patients

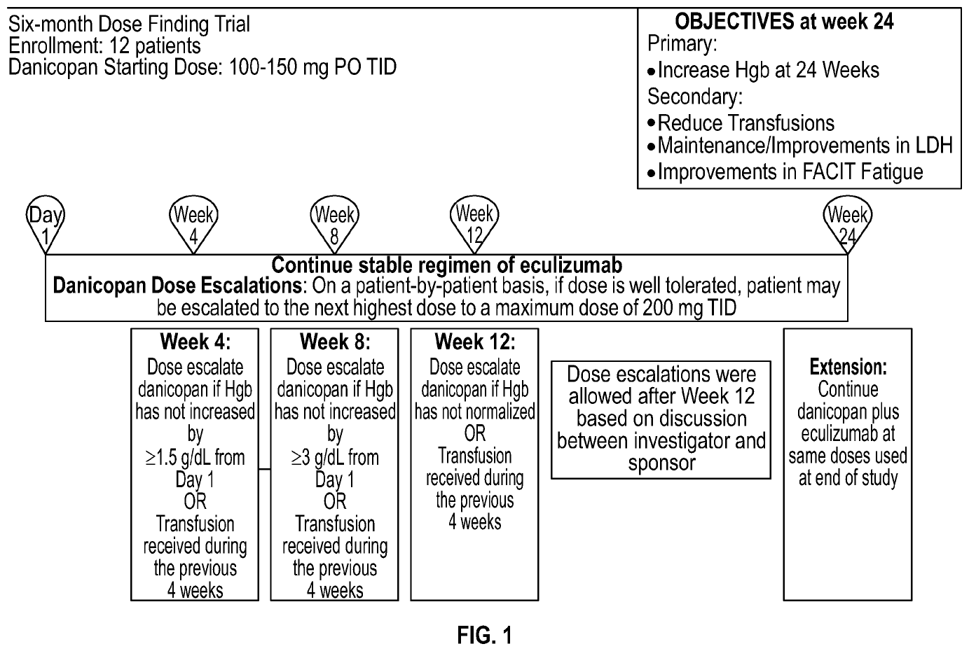

Twelve adult PNH patients up to age 65 who exhibited a sub-optimal response to SOURIS® (eculizumab) (i.e., hemoglobin [Hgb]<10 g/dL and transfusion dependent [≥1 RBC transfusion ≤12 weeks of screening]) received oral danicopan (ACH-4471; ACH-0144471) 100-150 mg three times a day (TID) in addition to continuation of their current SOURIS® (eculizumab) regimen as show in FIG. 1, with dose escalation based on response to 200 mg TID. Key inclusion and exclusion criteria are set forth in Table 2.

TABLE 2
Key Inclusion and Exclusion Criteria

| Key Inclusion Criteria | Key Exclusion Criteria |
|---|---|
| RBC-transfusion-dependent: ≥21 RBC | History of organ/stem cell/marrow transplant |
| transfusion ≤ 12 weeks prior to screening | Current evidence of bone marrow failure or |
| Anemia (Hgb < 10 g/dL) with adequate | aplastic anemia requiring treatment |
| reticulocytosis | eGFR530 mL/min/1.73 m2 and/or dialysis |
| Stable regimen of eculizumab: approved dose | Laboratory abnormalities |
| higher ≥ 24 weeks prior to entry without | ALP >1.5 × ULN, ANC <1,000/μL, ALT |
| change in regimen ≤ 8 weeks | >1.5 × ULN |
| Documentation of or willingness to receive | Direct bilirubin >1.5 × ULN (unless 2° |
| required vaccinations and antibiotic | to extravascular hemolysis) |
| prophylax: | Prior history or current evidence of biliary |
| | cholestasis |
| | Presence of Gilbert’s syndrome or personal |
| | family history of same |

Specifically, patients received oral danicopan at a starting dose of 100 mg or 150 mg three times a day and were instructed to take doses approximately the same time each day and as close as possible to 8 hours apart. All doses were taken approximately 15-30 minutes after completion of a meal or snack. Dose escalations, to a maximum of 200 mg three times a day, were based on safety and hemoglobin values and permitted at 4-week intervals through week 12 at 50 mg increments. If the patient had not already reached the 200 mg three times daily maximal dose by week 12, escalation was permitted if clinically indicated. Patients continued their pre-existing regimen of SOLIRIS® (eculizumab) throughout the study. Switching SOLIRIS® (eculizumab) to another C5 inhibitor was not permitted during the 24-week treatment period. Patients completing treatment with clinical benefit entered a long-term extension phase.

2. Endpoints

The primary objective of the study was to evaluate the efficacy of danicopan in addition to their standard of care SOLIRIS® (eculizumab) based on the increase in Hgb relative to baseline at treatment week (TW) 24. Secondary objectives included the reduction of RBC units transfused during the 24 weeks of danicopan compared to the 24 weeks prior to danicopan treatment, as well as the percentage of patients who were RBC transfusion-independent during the 24 weeks of danicopan treatment and the change from baseline in lactate dehydrogenase (LDH) at TW24. Additional endpoints included the effect of danicopan on complement biomarkers and assessment of fatigue, assessed with the FACIT-Fatigue instrument, wherein total scores on this instrument range from 0 to 52 with the higher scores indicating an improvement (see, e.g., Cella D, et al., Cancer. 2002; 94(2): 528-538). General safety, tolerability, pharmacokinetics and pharmacodynamics of danicopan were measured. After completing 24 weeks of treatment, patients entered a long-term extension.

3. Pharmacokinetic and Pharmacodynamic Methods

Plasma danicopan concentrations were determined with protein precipitation by addition of 0.15% formic acid in acetonitrile to a 75 μL plasma aliquot (K2EDTA as anticoagulant), followed by liquid chromatography/tandem mass spectrometry in positive ionization mode using a deuterated internal standard for quantitation. Ion transitions of 580.2 to 360.2 amu and 587.2 to 362.2 amu were monitored for danicopan and internal standard, respectively.

Pharmacodynamics were determined by measuring serum AP activity with AP hemolysis assay. Serum CP activity, Plasma Bb concentration, serum FD, and C3 concentrations were also monitored. Complement tests were conducted by a central lab using commercial kits, with the exception of AP hemolysis assay which was conducted internally for exploratory purposes. At each AP hemolysis run, a single normal human serum sample was included in addition to the patient serum samples so hemolysis values of individual patient serum samples could be standardized to the hemolysis value of the normal human serum sample. Finally, PNH clone size and C3 fragment deposition on erythrocytes was measured using flow cytometry with FITC conjugated anti-human C3d antibody.

4. Statistical Methods

Descriptive statistics are provided for biochemical, quality-of-life measurements, and transfusion data. Continuous variables are summarized with mean, median, minimum and maximum values. Categorical variables, e.g., transfusion-independent, are summarized with counts and percentages. Missing values were not imputed.

Transfusion frequency and amount were evaluated via annualized rates and units, respectively. The average intensive rate of transfusion frequency before treatment was calculated by summing the number of transfusion events in the 52 weeks prior to Screening plus days from Screening to Day 1 of dosing from 10 patients and dividing this sum by the patients' total exposure times. The average intensive rate in the 24 weeks post-treatment was calculated with the same method. The ratio of these two intensive rates (post vs. pre) was used to quantify the treatment effect. Standard statistical analysis for comparing the two intensity rates (post- vs pre-treatment) was conducted. The reduction in the amount transfused was evaluated via the annualized units transfused using the same procedure.

B. Results

1. Patient Characteristics

Twelve patients were enrolled and received at least one dose of danicopan. One discontinued after 2 doses, due to a serious adverse event of worsening of an underlying condition (pulmonary hypertension/edema), considered unlikely related to danicopan. This patient had pre-existing pulmonary hypertension (both valvular and as a consequence of their PNH) and their cardiac medications had been changed a few days before initiation of study drug. The serious adverse event was considered unlikely related to the study drug and the patient's data were excluded from this analysis.

Eleven patients completed treatment. The treatment regimen is presented in Table 3. The results are presented in Table 4. Median age was 42.5 years and mean C5 treatment duration prior to dosing was 36 months. All patients were on a stable regimen of SOLIRIS® (eculizumab) with 8 patients receiving the approved dose of 900 mg intravenously every 14 days. Two patients were on 1200 mg of SOLIRIS® (eculizumab) and one patient on 1500 mg every 14 days.

Patients had slightly elevated LDH levels at entry despite stable SOLIRIS® (eculizumab) therapy. Patients were anemic with a mean Hgb of 7.9 mg/dL (SD±1.5 g/dL) and all but one patient had a history of RBC transfusions with a mean of 3.4 transfusions (mean 5.8 units) in the 24 weeks prior to screening. The patient without a transfusion history did not accept transfusions due to religious objections; she started with a baseline Hgb of 5.0 g/dL (Patient A, Table 4) and a diagnosis of genetically confirmed hereditary elliptocytosis.

TABLE 3
Treatment Regimen

| | Baseline | Week 24 |
|---|---|---|
| | N = 11 | N = 11 |
| | Mean (SD) | Mean (SD) |
| Oral Danicopan TID | 100 mg (n = 9) | 100 mg (n = 2) |
| | 150 mg (n = 2) | 150 mg (n = 6) |
| | | 200 mg (n = 3) |
| Eculizumab IV Q2WK | 900 mg (n = 7) | 900 mg (n = 8) |
| | 1200 mg (n = 3) | 1200 mg (n = 2) |
| | 1500 mg (n = 1) | 1500 mg (n = 1) |

TABLE 4
Key Clinical Parameters at Baseline, Week 12 and Week 24

| | Baseline | | Week 12 | | Week 24 | |
|---|---|---|---|---|---|---|
| A: 41 y/o F | | | | | | |
| Danicopan dose (oral) | 100 | mg TID | 150 | mg TID | 150 | mg TID |
| Eculizumab dose (IV) | 900 | mg q14d | 900 | mg q14d | 900 | mf q14d |
| Hgb (g/dL) | 5.00 | | 7.70 | | 8.50 | |
| LDH (×ULN) | 1.7 | | 1.2 | | 1.1 | |
| Reticulocytes (10\hat{ }9/μL) | 159 | | 121 | | 112 | |
| Total bilirubin (mg/dL) | 2.14 | | 1.80 | | 2.40 | |
| Direct bilirubin (mg/dL) | 0.44 | | 0.33 | | 0.48 | |
| PNH RBC clone size (%) | 41 | | 83 | | —^{††} | |
| C3d⁺ PNHRBCs (%) | 6.30 | | —^{†} | | 2.20 | |
| FACIT-Fatigue* | 33 | | 48 | | 52 | |
| B: 51 y/o, F | | | | | | |
| Danicopan dose (oral) | 100 | mg TID | 100 | mg TID | 100 | mg TID |
| Eculizumab dose (IV) | 1200 | mg q14d | 900 | mg q14d | 900 | mg q14d |
| Hgb (g/dL) | 9.80 | | 11.8 | | 13.3 | |
| LDH (×ULN) | 1.0 | | 0.8 | | 0.9 | |
| Reticulocytes (10\hat{ }9/μL) | 250 | | 123 | | 162 | |
| Total bilirubin (mg/dL) | 1.24 | | 0.89 | | 0.81 | |
| Direct bilirubin (mg/dL) | 0.29 | | 0.18 | | 0.20 | |
| PNH RBC clone size (%) | 80 | | 89 | | —^{††} | |
| C3d⁺ PNHRBCs (%) | 8.20 | | —^{†} | | 1.00 | |
| FACIT-Fatigue* | 45 | | 49 | | 48 | |
| C: 67 y/o, M | | | | | | |
| Danicopan dose (oral) | 150 | mg TID | 150 | mg TID | 150 | mg TID |
| Eculizumab dose (IV) | 900 | mg q14d | 900 | mg q14d | 900 | mg q14d |
| Hgb (g/dL) | 7.60 | | 9.00 | | 9.70 | |
| LDH (×ULN) | 0.8 | | 0.9 | | 0.9 | |
| Reticulocytes (10\hat{ }9/μL) | 141 | | 97.0 | | 87.0 | |
| Total bilirubin (mg/dL) | 1.03 | | 0.580 | | 0.580 | |
| Direct bilirubin (mg/dL) | 0.24 | | 0.16 | | 0.16 | |
| PNH RBC clone size (%) | 22 | | 59 | | 51 | |
| C3d⁺ PNHRBCs (%) | 17.5 | | 18.6 | | 16.7 | |
| FACIT-Fatigue* | 17 | | 36 | | 26 | |
| D: 29 y/o, F | | | | | | |
| Danicopan dose (oral) | 150 | mg TID | 150 | mg TID | 150 | mg TID |
| Eculizumab dose (IV) | 900 | mg q14d | 900 | mg q14d | 900 | mg q14d |
| Hgb (g/dL) | 10.4 | | 11.7 | | 11.5 | |
| LDH (×ULN) | 1.1 | | 1.2 | | 0.9 | |
| Reticulocytes (10\hat{ }9/μL) | 191 | | 56.0 | | 56.0 | |
| Total bilirubin (mg/dL) | 2.26 | | 0.700 | | 0.650 | |
| Direct bilirubin (mg/dL) | 0.43 | | 0.15 | | 0.17 | |
| PNH RBC clone size (%) | 52 | | 99 | | 100 | |
| C3d⁺ PNHRBCs (%) | 15.9 | | 0.200 | | 5.20 | |
| FACIT-Fatigue* | 18 | | 41 | | 41 | |
| E: 22 y/o, F | | | | | | |
| Danicopan dose (oral) | 100 | mg TID | 150 | mg TID | 150 | mg TID |
| Eculizumab dose (IV) | 1200 | mg q14d | 1200 | mg q14d | 1200 | mg q14d |
| Hgb (g/dL) | 8.60 | | 7.60 | | 9.40 | |
| LDH (×ULN) | 1.2 | | 1.3 | | 1.2 | |
| Reticulocytes (10\hat{ }9/μL) | 120 | | 179 | | 90.0 | |
| Total bilirubin (mg/dL) | 2.35 | | 2.22 | | 1.75 | |
| Direct bilirubin (mg/dL) | 0.72 | | 0.40 | | 0.60 | |
| PNH RBC clone size (%) | 21 | | 37 | | 53 | |
| C3d⁺ PNHRBCs (%) | 21.3 | | 11.8 | | 4.90 | |
| FACIT-Fatigue* | 42 | | 25 | | 38 | |
| F: 44 y/o, F | | | | | | |
| Danicopan dose (oral) | 100 | mg TID | 150 | mg TID | 150 | mg TID |
| Eculizumab dose (IV) | 900 | mg q14d | 900 | mg q14d | 900 | mg q14d |
| Hgb (g/dL) | 7.20 | | 9.30 | | 10.6 | |
| LDH (×ULN) | 0.9 | | 0.9 | | 0.9 | |
| Reticulocytes (10\hat{ }9/μL) | 405 | | 243 | | 253 | |
| Total bilirubin (mg/dL) | 3.93 | | 1.50 | | 2.95 | |
| Direct bilirubin (mg/dL) | 0.82 | | 0.49 | | 0.78 | |
| PNH RBC clone size (%) | 95 | | 99 | | 98 | |
| C3d⁺ PNHRBCs (%) | 71.0 | | 48.0 | | 53.4 | |
| FACIT-Fatigue* | 9 | | 48 | | 52 | |
| G: 35 y/o, F | | | | | | |
| Danicopan dose (oral) | 100 | mg TID | 100 | mg TID | 100 | mg TID |
| Eculizumab dose (IV) | 1500 | mg q14d | 1500 | mg q14d | 1500 | mg q14d |
| Hgb (g/dL) | 7.10 | | 9.40 | | 9.10 | |
| LDH (×ULN) | 0.6 | | 0.5 | | 0.9 | |
| Reticulocytes (10\hat{ }9/μL) | 262 | | 200 | | 239 | |
| Total bilirubin (mg/dL) | 1.20 | | 1.90 | | 1.25 | |
| Direct bilirubin (mg/dL) | 0.39 | | 0.50 | | 0.39 | |
| PNH RBC clone size (%) | 62.8 | | 90.4 | | 96.0 | |
| C3d⁺ PNHRBCs (%) | 80.0 | | 29.4 | | 57.0 | |
| FACIT-Fatigue* | 28 | | 44 | | 38 | |
| H: 52 y/o, F | | | | | | |
| Danicopan dose (oral) | 100 | mg TID | 200 | mg TID | 200 | mg TID |
| Eculizumab dose (IV) | 900 | mg q14d | 900 | mg q14d | 900 | mg q14d |
| Hgb (g/dL) | 7.70 | | 7.30 | | 7.50 | |
| LDH (×ULN) | 1.5 | | 1.8 | | 1.4 | |
| Reticulocytes (10\hat{ }9/μL) | 238 | | 206 | | 169 | |
| Total bilirubin (mg/dL) | 2.18 | | 2.04 | | 1.57 | |
| Direct bilirubin (mg/dL) | 0.78 | | 0.53 | | 0.52 | |
| PNH RBC clone size (%) | 19 | | 71 | | —^{††} | |
| C3d⁺ PNHRBCs (%) | 38.8 | | 14.9 | | 22.8 | |
| FACIT-Fatigue* | 47 | | 49 | | 49 | |
| I: 50 y/o, F | | | | | | |
| Danicopan dose (oral) | 100 | mg TID | 100 | mg TID | 200 | mg TID |
| Eculizumab dose (IV) | 900 | mg q14d | 900 | mg q14d | 900 | mg q14d |
| Hgb (g/dL) | 7.70 | | 10.8 | | 11.5 | |
| LDH (×ULN) | 0.9 | | 0.9 | | 0.9 | |
| Reticulocytes (10\hat{ }9/μL) | 200 | | 110 | | 96 | |
| Total bilirubin (mg/dL) | 0.56 | | 0.34 | | 0.32 | |
| Direct bilirubin (mg/dL) | 0.23 | | 0.18 | | 0.18 | |
| PNH RBC clone size (%) | 73 | | 95 | | .tt | |
| C3d⁺ PNHRBCs (%) | 13.6 | | 0.400 | | 0.100 | |
| FACIT-Fatigue* | 48 | | 50 | | 49 | |
| J: 19 y/o, M | | | | | | |
| Danicopan dose (oral) | 100 | mg TID | 100 | mg TID | 200 | mg TID |
| Eculizumab dose (IV) | 1200 | mg q14d | 1200 | mg q14d | 1200 | mg q14d |
| Hgb (g/dL) | 7.80 | | 10.2 | | 11.0 | |
| LDH (×ULN) | 1.3 | | | | 1.2 | |
| Reticulocytes (10\hat{ }9/μL) | 258 | | 292 | | 152 | |
| Total bilirubin (mg/dL) | 3.24 | | —^{††} | | 1.14 | |
| Direct bilirubin (mg/dL) | 0.68 | | | | 0.23 | |
| PNH RBC clone size (%) | 60 | | 99 | | 99 | |
| C3d⁺ PNHRBCs (%) | 34.3 | | 59.5 | | 28.7 | |
| FACIT-Fatigue* | 49 | | 51 | | 51 | |
| K: 57 y/o, F | | | | | | |
| Danicopan dose (oral) | 100 | mg TID | 100 | mg TID | 150 | mg TID |
| Eculizumab dose (IV) | 900 | mg q14d | 900 | mg q14d | 900 | mg q14d |

| Hgb (g/dL) | 8.40 | | 9.40 | 11.5 | |
|---|---|---|---|---|---|
| LDH (×ULN) | 0.9 | | 1.2 | 1.1 | |
| Reticulocytes (10\hat{ }9/μL) | 188 | | 88.0 | 69.0 | |
| Total bilirubin (mg/dL) | 3.76 | | 1.85 | 1.41 | |
| Direct bilirubin (mg/dL) | 0.64 | | 0.39 | 0.31 | |
| PNH RBC clone size (%) | 55 | | 84 | 91 | |
| C3d⁺ PNHRBCs (%) | 20.2 | | 44.2 | 6.30 | |
| FACIT-Fatigue* | 33 | | 46 | 50 | |
| Descriptive Statistics: | | | | | |
| Mean (SD); N | | | | | |
| Hgb (g/dL) | | | | | |
| LDH (×ULN) | 1.06 | (0.321), 11 | | 1.04 | (0.181), 11 |
| Reticulocytes (10\hat{ }9/μL) | 219 | (78.1), 11 | | 135 | (66.3), 11 |
| Total bilirubin (mg/dL) | 2.17 | (1.118), 11 | | 1.35 | (0.798), 11 |
| Direct bilirubin (mg/dL) | 0.51 | (0.220), 11 | | 0.37 | (0.207), 11 |
| PNH RBC clone size (%) | 54 | (24.9), 11 | | 84 | (22.1), 7 |
| C3d⁺ PNHRBCs (%)^{$} | 22.2 | (6.3-80.0), 11 | | 6.68 | (0.100-57.0), 11 |
| FACIT-Fatigue* | 34 | (14.1), 11 | | 45 | (8.2), 11 |

F-female;
Hgb-hemoglobin;
IV-intravenous;
LDH-lactic acid dehydrogenase;
M-male;
PNH-paroxysmal nocturnal hemoglobinuria;
q14d-every 14 days;
RBCs-red blood cells;
TID-three times a day.
*Scores based on the Functional Assessment of Chronic Illness Therapy Fatique (FACIT)-Fatigue Scale V4. Score range 0-52. A score of less than 30 indicates severe fatique.
^{†}C3 fragment deposition wasn’t tested at Week 12 for this subject. No data entry.
^{††}Stability of the sample was interrupted
^{§}N = 7; for four patients, samples were out of stability range.
^{$}Geometric mean (range)

2. Study Disposition

Nine patients started danicopan at 100 mg every 8 hours and two started at 150 mg every 8 hours. Of the patients starting 100 mg, one patient was titrated to 200 mg every 8 hours and six were titrated to 150 mg every 8 hours with two of the 150 mg patients titrating to 200 mg every 8 hours by the end of the trial. All patients maintained their SOLIRIS® (eculizumab) regimen throughout the study, except for one patient in the United States who had their SOLIRIS® (eculizumab) dose decreased by their insurance company (from 1200 mg to 900 mg) prior to week 16 due to Hgb improvement. One patient discontinued after 2 doses of danicopan due to a serious adverse event unlikely related to danicopan as described above, leaving 11 patients who reached the primary endpoint at treatment and are included in the final analysis.

3. Pharmacokinetics

During intensive pharmacokinetic sampling days for 11 patients on Week 7, the mean (% CV) values of danicopan at steady state for Cₘₐₓ, Tₘₐₓ and AUC_{(0-8 hr)} were 432 (37) ng/mL, 2.14 (33) hr and 1806 (37) ng·hr/mL, respectively. The mean (% CV) C_{trough} value was 105 (57) ng/mL during these intensive sampling days. Sampling at through times over the rest of the study duration under less controlled conditions yielded a mean (% CV) C_{trough} value of 150 (59) ng/mL. These results are consistent with pharmacokinetic values of danicopan observed in healthy volunteer studies.

4. Clinical Efficacy

Benefits were observed in multiple laboratory markers of PNH, shown in Table 4. There was a mean increase in Hgb of 2.4 g/dL at 24 Weeks of treatment. This treatment effect on hemoglobin appeared by week 2 in most patients and was maintained for the duration of the study.

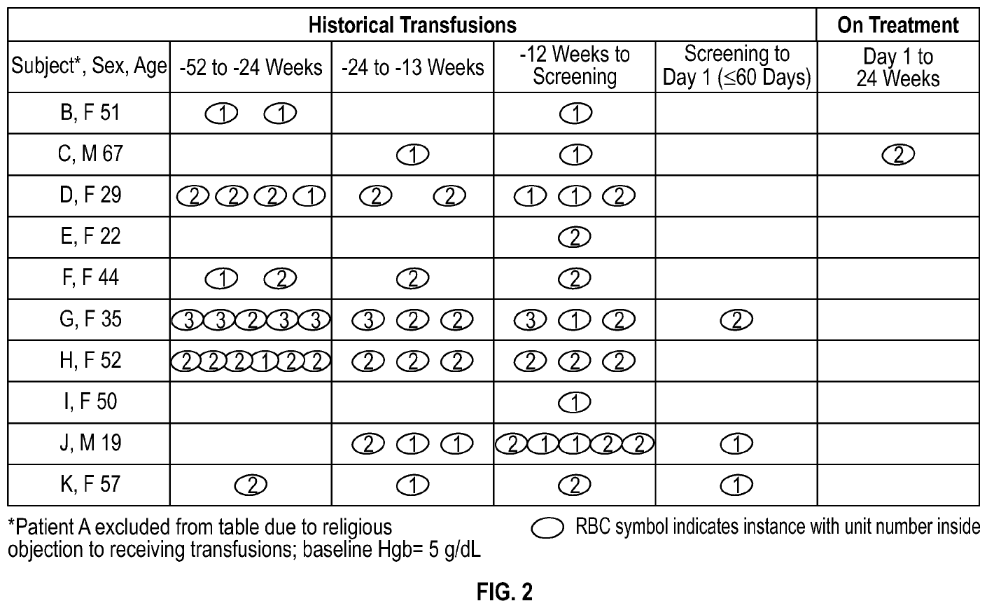

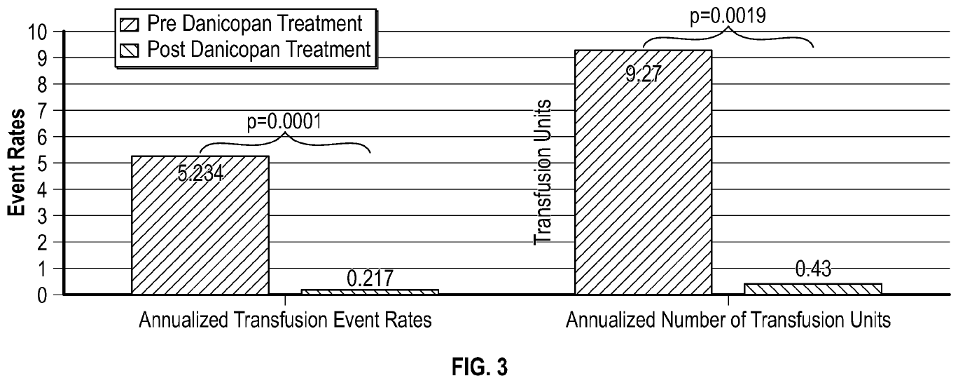

In addition to the observed rise in hemoglobin in patients receiving danicopan, a clinically meaningful reduction in RBC transfusion needs over the 24-week treatment period was demonstrated, as shown in FIG. 2. Among the ten patients with a 52-week transfusion history up to the time of screening and the time between screening and day of dosing (Day 1), 57 transfusions were administered totaling 101 units of packed red blood cells (PRBC) based on historical data. Note, one patient (Patient A) was excluded from the analysis because any transfusion was rejected due to religious objection. Since commencing treatment with danicopan, only one patient received a single transfusion through week 24, totaling 2 units, which was administered during a hospitalization for pneumonia that was considered by the investigator unlikely related to danicopan. The averages of the annualized rates of transfusion events are 5.234 for pre-danicopan and 0.217 for post-danicopan with a ratio (post- vs. pre-danicopan) of 0.042 (95% CI=0, 0.176; p=0.0001), demonstrating a highly statistically significant 95.8% reduction in transfusion frequency with danicopan (see FIG. 3). The averages of the annualized transfusion units are 9.27 for pre-danicopan and 0.43 for post-danicopan with a ratio (post- vs pre-danicopan) of 0.047 (95% CI=0, 0.224, p=0.0019), also reflecting a highly significant reduction in the amount of transfusion units due to the addition of danicopan (see FIG. 3).

In addition to improvements in hemoglobin and transfusion needs, improvements in clinically relevant parameters were observed (Table 4). Patients receiving danicopan in the study continues to experience further improvements in LDH relative to the upper limit of normal and significant improvements in absolute reticulocyte counts. Reduction in both total and direct bilirubin measures in patients with elevations at baseline were also observed in the study.

This study utilized the validated quality of life instrument FACIT-Fatigue Scale (Version 4). This self-reporting instrument measures the severity of fatigue being experienced on a scale of 0 to 52 where a score of less than 30 represents severe fatigue and higher scores indicate improvement in fatigue. A 3-point change is clinically meaningful on this scale. A 10-point or greater change is highly significant on this scale. FACIT-Fatigue scores were reported, with a mean increase of 11 points at 24 Weeks relative to the Baseline on SOLIRIS® (eculizumab) (Table 4). The greatest improvement was observed in Patient F (a female 44-year-old subject), who experienced a 43 point improvement from Baseline to Week 24 (i.e., 9 points at Baseline compared to 52 points at Week 24). This constitutes an upper limit of the disclosure.

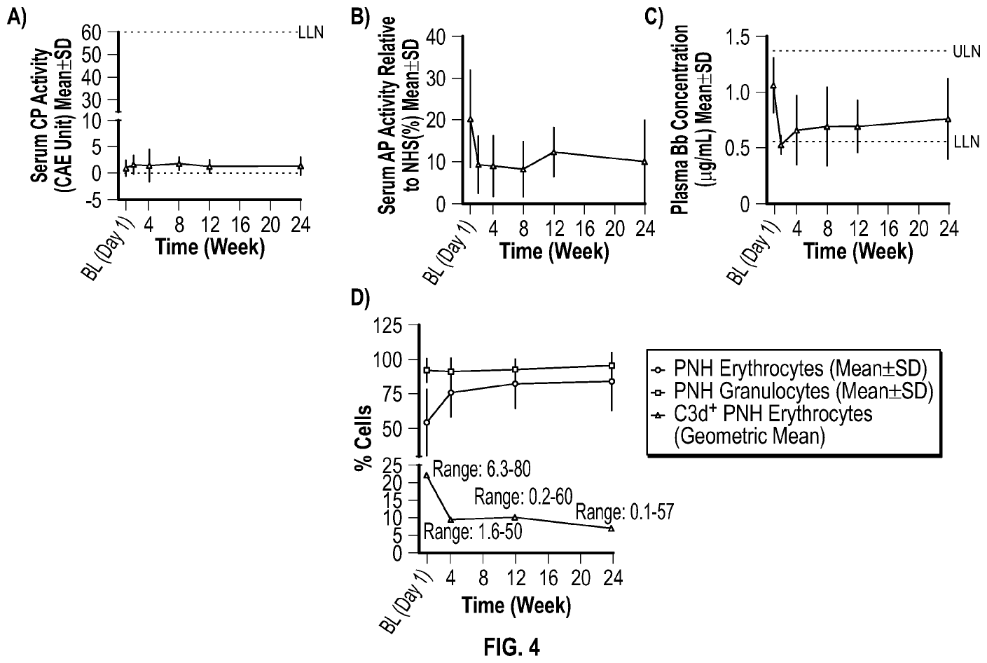

Complement biomarkers were monitored during the course of the study. Serum FD and C3 concentrations were normal at baseline and had little change during danicopan treatment (data not shown). The inhibition of CP activity was near complete at baseline, indicating the presence of little free serum C5 (FIG. 4A). This persisted throughout the study. In contrast, residual AP activity was detected with AP hemolysis assay at baseline and was reduced after dosing danicopan (FIG. 4B). In parallel, plasma Bb level was reduced after dosing with danicopan as well (FIG. 4C).

Consistent with a previous report from Hill et al. assessing C3 opsonization in PNH patients treated with SOLIRIS® (eculizumab), the percentage of PNH RBCs opsonized with C3 fragments (i.e., % C3d⁺ PNH RBCs) was high (geometric mean 22%; range 6.3-80%) at baseline as a consequence of C3 fragment accumulation on PNH RBCs having survived from intravascular hemolysis in the presence of a C5 inhibitor (see, e.g., Hill et al., Haematologica. 2010; 95: 567-573). The addition of danicopan significantly decreased the percentage of PNH RBCs opsonized with C3 fragments (geometric mean 6.7%; range 0.1-57% at Week 24) (FIG. 4D). Concomitantly, the clone size of PNH RBCs increased from a 54±24.9% at baseline to 84±22.1% (mean±SD) at Week 24-week, approaching the clone size of PNH granulocytes which was high at baseline (mean±SD: 92±9.2%) and remained to be high during the study course (mean±SD: 95±7.2% at Week 24) (FIG. 4D).

5. Safety

Danicopan was generally well tolerated. Treatment emergent adverse events (TEAEs) reported by at least 2 patients are listed in Table 5 and 96% of treatment TEAEs were mild to moderate in severity. There were no discontinuations due to TEAEs.

All events were considered mild to moderate in severity except for the following 2 patients. One patient experienced a Grade 3 direct bilirubin elevation which occurred in concert with a grade 1 ALT elevation at Day 70. This patient had similar elevations in these parameters at baseline and during screening. Both adverse events resolved by Day 77. Danicopan dose was temporarily reduced and then re-escalated after event resolved. This patient completed the study. The investigator considered these events to be caused by breakthrough hemolysis because of the associated approximate doubling of LDH and a decrease in Hgb of 0.8 mg/dL.

The second patient experienced a severe adverse event of pneumonia at Day 145 requiring hospitalization which recovered on Day 152. This event evolved from viral bronchitis. This patient also had a history of neutropenia. This patient received a transfusion of 2 units of PRBC during the hospitalization at an institution separate from the trial center. Relationship to study drug considered unlikely. Danicopan dose was not changed and the patient completed study.

TABLE 5
Safety

| MedDRA Preferred Term Reported | | |
|---|---|---|
| by Greater than 10% of Patients | n (%) | |
| Number of Patients | 11 | (100) |
| Reporting a TEAE | | |
| Headache | 3 | (27) |
| Abdominal pain | 2 | (18) |
| Contusion | 2 | (18) |
| Cough | 2 | (18) |
| Fatigue | 2 | (18) |
| Nasopharyngitis | 2 | (18) |
| Nausea | 2 | (18) |
| Oropharyngeal | 2 | (18) |
| Pain in extremity | 2 | (18) |

C. Summary

C5 inhibition with SOLIRIS® (eculizumab) or ULTOMIRIS® (ravulizumab), the current standard of care, is an effective treatment approach for patients with PNH. While this treatment approach controls intravascular hemolysis and provides a dramatic improvement in overall survival, many patients remain anemic and some may continue to be transfusion dependent due to persistent extravascular hemolysis.

In the above-described clinical trial, when danicopan was added to background SOLIRIS® (eculizumab) therapy in these severely anemic patients (primarily due to extravascular hemolysis (EVH)), it resulted in a 2.4-gram mean increase in Hgb and a clinically and statistically significant reduction in RBC transfusion needs. The mean increase of 11 points in FACIT-Fatigue score is remarkable and highly significant as the patients came into this trial on a background of SOLIRIS® (eculizumab) therapy. The International PNH Registry has shown that fatigue is one of the most commonly patient reported symptoms in untreated patients with approximately 80% patients reporting fatigue in the past six months (see, e.g., Schrezenmeier H., et al., Haematologica. 2014; 99(5): 922-929). Fatigue is often assessed with the use of the FACIT-Fatigue scale in patients experiencing anemia, such as those with cancer and PNH. Treatment with SOLIRIS® (eculizumab) monotherapy has improved levels of fatigue in patients with PNH, measured by FACIT-Fatigue, as shown in the landmark trials SHEPHERD and TRIUMPH where scores significantly increased by 12.2 points and 6.4 points versus baseline, respectively (see, e.g., Brodsky R A, et al., Blood. 2008; 111(4): 1840-1847 and Hillmen P, et al., N. Engl. J. Med. 2006; 355:1233-1243). In this current trial, the addition of danicopan to SOLIRIS® (eculizumab) in patients that continued to be anemic, not only raised hemoglobin by over 2 g/dL but also demonstrates the potential impact the addition of danicopan can have on a patient's quality of life.

The addition of danicopan to SOLIRIS® (eculizumab) nearly eliminated the need for transfusions in most of the patients in this study. There was one patient that was enrolled in the trial that did not have a transfusion history due to religious objections. She entered the trial with a baseline hemoglobin of 5 g/dL on SOLIRIS® (eculizumab). The addition of danicopan to her SOLIRIS® (eculizumab) therapy raised her hemoglobin over 3 g/dL at 24-weeks and significantly improved her fatigue. She also carries a diagnosis of hereditary elliptocytosis, another hemolytic anemia for which danicopan would have no impact.

There were also meaningful improvements in bilirubin and reticulocytes. Total bilirubin was driven into normal range at Week 24 versus baseline and reticulocyte count was reduced to near normal at Week 24. Additionally, the PNH erythrocyte clone size approached that of PNH granulocytes demonstrating that PNH RBCs were being further protected from hemolysis with the addition of danicopan. The percentage of PNH RBCs opsonized with C3 fragments decreased from baseline to Week 24, which is supportive of the upstream AP mechanism of action of danicopan. Together these demonstrate that C3-mediated extravascular hemolysis is being prevented by danicopan, while retaining the control of MAC-mediated intravascular hemolysis.

In sum, proof of concept was established with danicopan in the treatment of PNH in addition to the standard of care SOLIRIS® (eculizumab). Danicopan was generally well tolerated and demonstrated meaningful improvement in Hgb, transfusion needs, FACIT-Fatigue, and other parameters of interest were achieved. This demonstrates that further benefit can be achieved in patients receiving standard of care with SOLIRIS® (eculizumab) by blocking the AP at factor D with danicopan. This benefit is likely due to the prevention of C3-mediated extravascular hemolysis, while controlling intravascular hemolysis. Danicopan targets an unmet need in PNH.

### Example 2: A Phase 3 Study of Danicopan as Add-on Therapy to a C5 Inhibitor in Patients with Paroxysmal Nocturnal Hemoglobinuria Who have Clinically Evident Extravascular Hemolysis (EVH)

A phase 3 clinical study is conducted in adult PNH patients (18+ years) who have clinically evident extravascular hemolysis (EVH) substantially according to the protocol set forth, the totality of disclosure in which is incorporated by reference herein.

A. Objectives

The main objective of the study is to evaluate the efficacy of danicopan (also known as “ALXN2040” and ACH 4471) as compared to an oral (tablet) placebo, as an add-on therapy to a C5 inhibitor (i.e., SOLIRIS® or ULTOMIRIS®) at 12 weeks. The main endpoint is change in hemoglobin (Hgb) relative to baseline after 12 weeks of treatment with danicopan compared to placebo.

Key secondary objectives include assessing the proportion of patients with transfusion avoidance, the change from baseline in Functional Assessment of Chronic Illness Therapy (FACIT) fatigue scores, and the change from baseline in absolute reticulocyte count.

evaluating: (1) the efficacy of danicopan as compared to placebo as add-on therapy to a C5 inhibitor on transfusion avoidance at 12 weeks (i.e., proportion of patients with transfusion avoidance (TA), defined as patients who remain transfusion-free and do not require a transfusion as per protocol-specified guidelines through Week 12), (2) the effect of danicopan as compared to placebo as add-on therapy to a C5 inhibitor on FACIT Fatigue scores at 12 weeks of treatment (i.e., change from baseline in FACIT Fatigue scores at Week 12), and (3) the effect of danicopan as compared to placebo as add-on therapy to a C5 inhibitor on absolute reticulocyte count (i.e., change from baseline in absolute reticulocyte count at Week 12).

Additional objectives include evaluating: (1) the efficacy of danicopan as add-on therapy to a C5 inhibitor on transfusion requirements at 24 weeks for those patients receiving 24 weeks of Danicopan (i.e., change in the number of red blood cell (RBC) units transfused and transfusion instances during the 24 weeks of treatment with danicopan compared to the 24 weeks prior to initiation of treatment with danicopan and percentage of patients who have transfusion avoidance through 24 weeks of treatment), (2) the efficacy of danicopan as compared to placebo as add-on therapy to a C5 inhibitor on transfusion requirements at 12 weeks (i.e., change in the number of RBC units transfused and transfusion instances during the 12 weeks of treatment with danicopan compared to the 12 weeks while receiving placebo), (3) the effect of danicopan as add-on therapy to a C5 inhibitor on FACIT Fatigue scores for 24 weeks of treatment (i.e., change from baseline in FACIT Fatigue scores at Week 24 in all patients). Further objectives include assessing: (1) the efficacy of danicopan as add-on therapy to a C5 inhibitor on hemoglobin stabilization (i.e., percentage of patients with hemoglobin stabilization during the last 12 weeks of treatment in patients receiving 24 weeks of danicopan) and (2) additional laboratory markers relevant in PNH patients (i.e., change from baseline of danicopan treated patients compared to placebo in total and direct bilirubin at 12 weeks, changes in PNH RBC clone size, C3 fragment deposition on PNH RBCS, and measures of alternate pathway activity at 12 weeks of treatment with danicopan compared to placebo, changes in lactate dehydrogenase (LDH) and classical pathway activity at 12-weeks, and percentage of patients with hemoglobin normalization at 12 weeks and 24 weeks).

Exploratory objectives include assessing Patient-Reported Outcomes (PRO) and other health-related quality of life (QoL) measures during 24 weeks of treatment (i.e., change from baseline relative to placebo in Three-level EuroQoL 5 dimensions (EQ-5D-3L) scores at Week 12, change from baseline in EQ-5D-3L scores at Week 24, change from baseline relative to placebo in European Organization for Research and Treatment of Cancer (EORTC) Quality of Life Questionnaire-Core 30 Scale (QLQ-C30) at Week 12, change from baseline in EORTC-QLQ-C30 scale at Week 24, change from baseline relative to placebo in Work Productivity and Activity Impairment Questionnaire: General health (WPAI: SHP, V2.0) Week 12, change from baseline in WPAI: SHP scores at Week 24, change from baseline relative to placebo in Healthcare Resource Utilization (HRU) at Week 12, and change from baseline in HRU scores at Week 24).

Safety objectives include evaluating: (1) the safety and tolerability of 24 weeks of treatment with danicopan as add-on therapy to a C5 inhibitor (i.e., Incidence of Treatment-emergent adverse events (TEAEs), serious adverse events (SAEs), laboratory abnormalities, and events leading to discontinuation of study drug during 12-week blinded and subsequent 12-week open label treatment periods), and (2) the safety and tolerability of with danicopan as add-on therapy to a C5 inhibitor during LTE Period (i.e., Incidence of Treatment-emergent adverse events (TEAEs), serious adverse events (SAEs), laboratory abnormalities, and events leading to discontinuation of study drug).

B. Overall Design

This is a multiple-region, randomized, double-blind, placebo controlled, multiple-dose, Phase 3 study in patients with PNH who have clinically evident EVH on a C5 inhibitor (eculizumab or ravulizumab). This study includes approximately 84 patients who are receiving C5 inhibitor therapy according to the usual dose and schedule. These are enrolled and treated with danicopan or placebo (2:1 ratio).

Randomization is stratified by transfusion history (i.e., >2 or ≤2 transfusions within 6 months of Screening) and Hgb (i.e., <8.5 g/dL and ≥8.5 g/dL) at Screening, and Japanese patients (defined as patients enrolled from Japan)/non-Japanese patients. Patients are randomized to danicopan three times per day (tid) or placebo three times per day, in a 2:1 ratio, for 12 weeks (Treatment Period 1) in addition to their C5 inhibitor therapy (eculizumab or ravulizumab). At Week 12, patients randomized to receive placebo are switched to danicopan for an additional 12 weeks (Treatment Period 2) and patients randomized to danicopan continue on danicopan for an additional 12 weeks, while remaining on the ongoing C5 inhibitor therapy. At the end of the treatment periods (Week 24), patients can enter the Long-Term Extension (LTE) Period and continue to receive danicopan plus their C5 inhibitor therapy.

In this study, patients have been on a C5 inhibitor therapy for a time period sufficient to receive the full benefit of the therapy yet still remain anemic. Prolonged therapy with a C5 inhibitor alone is not projected to have additional impact on their clinical response. Historical transfusion needs and pretransfusion hemoglobin levels are captured for the 52 weeks prior to the screening visit. These historical data are used to assess the efficacy and safety of combination therapy in this study.

The screening visit occurs no earlier than 4 weeks after a transfusion to minimize the effect of the transfusion on the screening Hgb level, which is used for stratification purposes. Patients are evaluated for history of vaccination. All patients are vaccinated against meningococcal infections within 3 years prior to, or at the time of, initiating study drug. Patients who initiate study drug treatment less than 2 weeks after receiving a meningococcal vaccine must receive treatment with appropriate prophylactic antibiotics until 2 weeks after vaccination.

The starting dose of danicopan or placebo is 150 mg three times per day. Any patient with alanine aminotransferase or direct bilirubin screening value >1.5×upper limit of normal (ULN) commence dosing at 100 mg three times per day. Patients with documented Gilbert's Syndrome are started at the recommended starting dose of 150 mg three times per day. A minimum of 4 weeks of treatment is required at each dose level before any subsequent escalation to the next dose level. Doses can be escalated in 50-mg increments to a maximum of 200 mg three times per day based on safety and clinical effect at protocol-specified time points (Weeks 4, 8, and 12). All dose escalations obtained after the Week 12 visit are made on a patient-by-patient basis. The maximum dose in Treatment Period 2 is 200 mg three times a day. Patients cannot switch from their Day 1 C5 inhibitor to any other C5 inhibitor during the first 24 weeks of the study but may do so during the LTE Period.

The C5 Inhibitor+Placebo Group is dose-escalated in the same manner as the C5 Inhibitor+Danicopan Group during the study to maintain the blind. After Week 12, the C5 Inhibitor+Placebo Group switches placebo to receive danicopan during Treatment Period 2.

All patients return to the clinic for safety and other assessments during the treatment periods and during the LTE Period as shown in Tables 6-8 of Example 3. Upon completion of Treatment Period 2 (Week 24), patients can enter the LTE Period for 1 year at the same danicopan dose they were receiving at Week 24, plus their C5 inhibitor therapy. During the LTE Period, patients can be dose escalated, to a maximum of 200 mg three times a day.

If a patient discontinues from the study, dosing of danicopan or placebo is tapered over 6 days (Taper Visit 1 and 2), and two Follow-up Visits are conducted approximately 14 days and 28 days after the last dose of study drug. Patients continue to receive their C5 inhibitor therapy at the same dose and interval that they were receiving during the taper and follow-up visits.

Patients are randomized to danicopan or placebo, in a 2:1 ratio, for 12 weeks (Treatment Period 1) in addition to their C5 inhibitor therapy. At Week 12, patients randomized to receive placebo are switched to danicopan for an additional 12 weeks (Treatment Period 2), and patients randomized to danicopan continue for an additional 12 weeks. At the end of the treatment periods (24 weeks), patients can enter the LTE Period at the same dose plus their C5 inhibitor therapy. Any patient discontinuing from the study at any time point should undergo a 6-day taper and follow-up for an additional 28 days.

The C5 inhibitor (eculizumab or ravulizumab) used in this study are considered a background therapy. If patients switch from eculizumab to ravulizumab after completion of 24 weeks of treatment, the new medication used in this manner is also considered a background therapy, as patients are required to be on a stable dose and interval of their C5 inhibitor for a prolonged period prior to study entry.

The following criteria is required for inclusion in the study:
1. Diagnosis of PNH
2. Clinically Evident Extravascular hemolysis (EVH) defined by:
   Anemia (Hgb≤9.5 g/dL) with absolute reticulocyte count ≥120×10⁹/L.
   At least 1 packed RBC or whole blood transfusion within 6 months prior to the start of the study
3. Receiving a C5 inhibitor for at least 6 months prior to Day 1 in this study at an approved dose (or higher) and with no change in dose or interval for 224 weeks preceding Day 1 in this study. For those patients who recently switched from eculizumab to ravulizumab, they must have received at least the loading dose and 3 maintenance doses (minimum of 24 weeks) of ravulizumab preceding Day 1.
4. Platelet count ≥30,000/4, without the need for platelet transfusions.
5. Absolute neutrophil counts ≥750/μL.
6. Documentation of vaccination for Neisseria meningitidis: All patients must be vaccinated against meningococcal infections within 3 years prior to, or at the time of, initiating study drug. Patients who initiate study drug treatment less than 2 weeks after receiving a meningococcal vaccine must receive treatment with appropriate prophylactic antibiotics until 2 weeks after vaccination.
7. Age 18 years or older (or greater than or equal to minimum adult age in accordance with local legal requirements)
8. Female patients of childbearing potential must agree to use a highly effective or acceptable method of contraception from the date of signing the informed consent to 30 days after their last dose of study drug. Female patients of childbearing potential must also have a negative serum pregnancy test during Screening and negative urine pregnancy test on Day 1.
9. Female patients with documented evidence of non-childbearing potential need not employ a method of contraception.
10. Nonsterile male patients must agree to use a highly effective or acceptable method of contraception with their partner(s) of childbearing potential from the first day of dosing to 90 days after their last dose of study drug. Males who are surgically sterile need not employ additional contraception. Males must agree not to donate sperm while enrolled in this study and for 90 days after their last dose of study drug.
11. Capable of giving signed informed consent, which includes compliance with the requirements and restrictions listed in the informed consent form and in this protocol.
12. Must have access to emergency medical care.

A patient is excluded from the study based on the following criteria:
1. History of a major organ transplant (e.g., heart, lung, kidney, liver) or hematopoietic stem cell transplantation (HSCT).
2. Patient's with known aplastic anemia or other bone marrow failure that requires HSCT or other therapies, including anti-thymocyte globulin and/or immunosuppressants
3. Received another investigational agent other than C5 inhibitors (eculizumab or ravulizumab) within 30 days or 5 half-lives of the investigational agent prior to study entry, whichever is greater.
1. Known or suspected complement deficiency.
2. Active bacterial or viral infection, a body temperature >38° C. on two consecutive daily measures, evidence of other infection, or history of any febrile illness within 14 days prior to first study drug administration.
3. History or presence of any clinically relevant co-morbidities that would make the patient inappropriate for the study (e.g., is likely to result in deterioration of the patient's condition, affect the patient's safety during the study, or confound the results of the study).
4. Laboratory abnormalities at screening, including:

ALP>2×ULN

ALT>2×ULN
Direct bilirubin >2×ULN (unless due to extravascular hemolysis, in the opinion of the Investigator) and patients with Gilbert's Syndrome are allowed into this study; however, documentation of Gilbert's Syndrome is required. If increased bilirubin is suggestive of Gilbert's syndrome, but the patient cannot provide documentation; then, the patient is tested for this condition (See below for details).
8. Any other clinically significant laboratory abnormality as judged by the Investigator that, in the opinion of the Principal Investigator, would make the patient inappropriate for the study or put the patient at undue risk.
9. Females who are pregnant, nursing, or planning to become pregnant during the study or within 90 days of study drug administration.
10. Current evidence of biliary cholestasis.
11. Evidence of human immunodeficiency virus, hepatitis B, or active hepatitis C infection at screening.
12. Estimated glomerular filtration rate (eGFR)<30 mL/min/1.73 m² and/or are on dialysis.
13. Hypersensitivity to the investigational drug (danicopan) or any of its excipients.

### Example 3

A multiple-region, randomized, double-blind, placebo controlled, multiple-dose, Phase 3 study was initiated in patients with PNH who have clinically evident EVH on a C5 Inhibitor (eculizumab or ravulizumab). This study includes approximately 84 patients who are receiving C5 inhibitor therapy according to the usual dose and schedule. Randomization is stratified by transfusion history (e.g., >2 or ≤2 transfusions within 6 months of Screening) and Hgb (e.g., <8.5 g/dL and ≥8.5 g/dL) at screening, and Japanese patients (defined as patients enrolled from Japan)/non-Japanese patients.

Patients are randomized to danicopan tid or placebo tid, in a 2:1 ratio, for 12 weeks (Treatment Period 1) in addition to their C5 inhibitor therapy (eculizumab or ravulizumab). At Week 12, patients randomized to receive placebo are switched to danicopan for an additional 12 weeks (Treatment Period 2) and patients randomized to danicopan are to continue on danicopan for an additional 12 weeks, while remaining on the ongoing C5 inhibitor therapy. At the end of the treatment periods (Week 24), patients may enter the Long-Term Extension (LTE) Period and continue to receive danicopan+their C5 inhibitor therapy.

In this study, PNH patients have been on a C5 inhibitor therapy for a time period sufficient to receive the full benefit of the therapy yet still remain anemic. Prolonged therapy with a C5 inhibitor alone is not projected to have additional impact on their clinical response. Historical transfusion needs and pretransfusion hemoglobin levels may be captured for the 52 weeks prior to the screening visit. These historical data may be used to assess the efficacy and safety of combination therapy in this study.

The screening visit is carried out no earlier than 4 weeks after a transfusion in order to minimize the effect of the transfusion on the screening Hgb level, which will be used for stratification purposes.

Patients will be evaluated for history of vaccination. All patients must be vaccinated against meningococcal infections within 3 years prior to, or at the time of, initiating study drug. Patients who initiate study drug treatment less than 2 weeks after receiving a meningococcal vaccine must receive treatment with appropriate prophylactic antibiotics until 2 weeks after vaccination. The starting dose of danicopan or placebo is 150 mg tid. Any patient with alanine aminotransferase or direct bilirubin screening value>1.5×upper limit of normal (ULN) will commence dosing at 100 mg tid. Patients with documented Gilbert's Syndrome are started at the recommended starting dose of 150 mg tid. A minimum of 4 weeks of treatment will be required at each dose level before any subsequent escalation to the next dose level. Doses may be escalated in 50-mg increments to a maximum of 200 mg tid based on safety and clinical effect at protocol-specified time points (Weeks 4, 8, and 12). All dose escalations obtained after the Week 12 visit are made on a patient-by-patient basis at the discretion of the Principal Investigator, in consultation with the Sponsor. The maximum dose in Treatment Period 2 is 200 mg tid. Patients may not switch from their Day 1 C5 inhibitor to any other C5 inhibitor during the first 24 weeks of the study but may do so during the LTE Period.

The C5 Inhibitor+Placebo Group are dose-escalated in the same manner as the C5 Inhibitor+Danicopan Group during the study to maintain the blind. After Week 12, the C5 Inhibitor+Placebo Group are switched placebo to receive danicopan during Treatment Period 2.

All patients will return to the clinic for safety and other assessments during the treatment periods and during the LTE Period as shown in Table 6 to Table 8.

Schedule of Assessments

TABLE 6
Schedule of Assessments Treatment Period 1: All Patients

| | Screening | Treatment 1₁ | | | | | | | | | |
|---|---|---|---|---|---|---|---|---|---|---|---|
| | Day −45 | Day | Wk | Wk | Wk | Wk | Wk | Wk | Wk | Wk | Post- |
| | to −1 | 1 | 1 | 2 | 3 | 4² | 6 | 8² | 10 | 12₂ | escalation³ |
| Clinic visit days | X | X | | X | | X | | X | | X | |
| Visiting healthcare | | | X | | X | | X | | X | | |
| assessment⁴ | | | | | | | | | | | |
| Vaccinations⁶ | X | | | | | | | | | | |
| Study drug dispensing⁷ | | X | | X | | X | | X | | X | |
| Clinical assessments | | | | | | | | | | | |
| Informed consent | X | | | | | | | | | | |
| Inclusion/exclusion criteria | X | X | | | | | | | | | |
| Review safety card | | X | X | X | X | X | X | X | X | X | X |
| Medical history | X | X | | | | | | | | | |
| Demographics | X | | | | | | | | | | |
| Vaccination history | X | | | | | | | | | | |
| Height | X | | | | | | | | | | |
| Physical exam⁸ | X | X | | X | | X | | X | | X | |
| Vital signs (including | X | X | | X | | X | | X | | X | |
| temperature)⁹ | | | | | | | | | | | |
| Weight | X | X | | X | | X | | X | | X | |
| 12-lead ECG (single) | X | | | | | | | | | X | |
| PRO and QoL | X | X | | X | | X | | | | X | |
| questionnaires¹⁴ | | | | | | | | | | | |
| RBC transfusion review | X | X | | X | | X | | X | | X | |
| AE/SAE | X | X | X | X | X | X | X | X | X | X | X |
| Concomitant medications/ | X | X | X | X | X | X | X | X | X | X | X |
| protocol restrictions | | | | | | | | | | | |

Laboratory assessments¹⁰

| | | | | | | | | | | | |
|---|---|---|---|---|---|---|---|---|---|---|---|
| FSH⁵ | X | | | | | | | | | | |
| HCV, Hbs Ag, HIV Ab | X | | | | | | | | | | |
| Erythropoietin | X | | | | | | | | | | |
| Sample for genetic | X | | | | | | | | | | |
| testing if needed | | | | | | | | | | | |
| (white blood cells) | | | | | | | | | | | |
| Hematology, chemistry, | X | X | X | X | X | X | X | X | X | X | X |
| and urinalysis¹¹ | | | | | | | | | | | |
| Pregnancy test¹² | X | X | | X | | X | | X | | X | |
| PT/PTT/INR, D-dimer | X | | | | | X | | | | X | |
| Free hemoglobin, | X | X | | X | | X | | X | | X | |
| haptoglobin | | | | | | | | | | | |
| Iron studies | X | | | | | | | | | X | |
| Direct Coombs | X | X | | | | X | | | | X | |
| Bb, APH | | X | | X | | X | | X | | X | |
| FD, C3, CP activity | | X | | | | X | | | | X | |
| Flow cytometry: clone size | X | X | | | | X | | | | X | |
| Flow cytometry: C3 | | X | | | | X | | | | X | |
| fragment deposition | | | | | | | | | | | |
| PK Sample¹³ | | X | | | | X | | | | X¹³ | |

AE = adverse event; AP = alternative pathway; APH = AP hemolysis; CP = classical pathway; ECG = electrocardiogram; FD = factor D; FSH = follicle-stimulating hormone; Hbs Ag = hepatitis B surface antigen; HCV = hepatitis C virus; HIV Ab = human immunodeficiency virus antibody; HRU = Healthcare Resource Utilization; INR = international normalized ratio; PK = pharmacokinetics; PT = prothrombin time; PTT = partial thromboplastin time; QoL = quality of life; RBC = red blood cell; SAE = serious adverse event.
¹Visit window is ±1 day for Weeks 1 through 12. A patient can discontinue from the study at any time and should complete all Week 24 assessments at final visit
²Weeks 4, 8, and 12 are potential dose escalation time points.
³If a dose escalation occurs, blood should be drawn for measurement of alanine aminotransferase, aspartate aminotransferase, γ-glutamyl transferase, and alkaline phosphatase by the visiting healthcare service or at the clinic 72 to 96 hours after escalation.
⁴The site will call patient within 1 to 3 days to confirm that visiting healthcare assessment occurred and assess AEs, SAEs, and concomitant medications.
⁵FSH for postmenopausal women.
⁶Female patients of childbearing potential receiving vaccinations or boosters must have a negative urine pregnancy test on the days of vaccination, before any vaccine or booster is administered.
⁷Patients will be provided with sufficient study drug to last until their next appointment. At dose escalation visits, patients will return to the clinic to be dispensed study drug and receive new dosing instructions.
⁸Full physical exam at Screening and Day 1. Brief physical exam at all other time points.
⁹Patients will be monitored for fever at every clinic visit and will monitor themselves in between visits.
¹⁰Patients must fast for 8 hours before blood draws at Screening, Day 1 and all Visiting Healthcare Assessments.
¹¹Patients should refrain from heavy exercise 24 hours before blood collection. Walking and light exercise are acceptable.
¹²Serum pregnancy test at screening. Urine pregnancy test for women of childbearing potential only. On Day 1, the pre-dose urine pregnancy test must be negative to continue. Any positive urine pregnancy test will be confirmed by a follow-up serum pregnancy test.
¹³Site will either obtain a pre-dose or post-dose sample depending on timing of clinic visit Actual sampling time and the most recent dose time prior to sample collection should be recorded. The sample should be collected at the same time of FD, C3, CP activity sample collection
¹⁴Refer to PRO and QoL questionnaires. HRU will be administered at day 1 and week 12

TABLE 7
Schedule of Assessments Treatment Period 2: All Patients

| | Wk | Wk | Wk | Wk | Wk | Wk | Wk | Wk | Post- |
|---|---|---|---|---|---|---|---|---|---|
| | 13 | 14 | 15 | 16 | 18 | 20 | 22 | 24/E | escalation² |
| Clinic visit days¹ | | X | | X | | X | | X | |
| Visiting healthcare assessement³ | X | | X | | X | | X | | |
| Vaccinations⁴ | Administer according to local/national guidelines | | | | | | | | |
| Study drug dispensing⁵ | | X | | X | | X | | X | |

Clinical assessments

| | | | | | | | | | |
|---|---|---|---|---|---|---|---|---|---|
| Review safety card | X | X | X | X | X | X | X | X | X |
| Brief Physical exam | | X | | X | | X | | X | |
| Vital signs (including temperature)⁶ | | X | | X | | X | | X | |
| Weight | | X | | X | | X | | X | |
| 12-lead ECG (single) | | | | | | | | X | |
| PRO¹¹ and QoL questionnaires | | X | | X | | | | X | |
| RBC transfusion review | | X | | X | | X | | X | |
| AE/SAE | X | X | X | X | X | X | X | X | X |
| Concomitant medications/protocol restrictions | X | X | X | X | X | X | X | X | X |

Laboratory assessments⁷

| | | | | | | | | | |
|---|---|---|---|---|---|---|---|---|---|
| Hematology, chemistry, and urinalysis⁸ | X | X | X | X | X | X | X | X | X |
| Pregnancy test⁹ | | X | | | | X | | X | |
| PT/PTT/INR, D-dimer | | | | X | | | | X | |
| Free hemoglobin, haptoglobin | | X | | X | | X | | X | |
| Iron studies | | | | | | | | X | |
| Direct Coombs | | | | X | | | | X | |
| Bb, APH | | X | | X | | X | | X | |
| FD, C3, CP activity | | | | X | | | | X | |
| Flow cytometry: clone size | | | | X | | | | X | |
| Flow cytometry: C3 fragment deposition | | | | X | | | | X | |
| PK Sample¹⁰ | | | | X | | | | X¹⁰ | |

AE = adverse event; AP = alternative pathway; APH = AP hemolysis; CP = classical pathway; ECG = electrocardiogram; EORTC-QLQ30 = European Organization for Research and Treatment of Cancer Quality of Life Questionnaire-Core 30 Scale; ET = early termination; FD = factor D; HRU = Healthcare Resource Utilization; INR = international normalized ratio; PK = pharmacokinetics; PT = prothrombin time; PTT = partial thromboplastin time; QoL = quality of life; RBC = red blood cell; SAE = serious adverse event; WAPI: SHP = Work Productivity and Activity Impairment Questionnaire: Specific Health Problem.
¹Visit window is ±1 day for Weeks 13 through 24.
²If a dose escalation occurs, blood should be drawn for measurement of alanine aminotransferase, aspartate aminotransferase, γ-glutamyl transferase, and alkaline phosphatase by the visiting healthcare service or at the clinic 72 to 96 hours after escalation.
³The site will call patient within 1 to 3 days to confirm that visiting healthcare assessment occurred and assess AEs, SAEs, and concomitant medications.
⁴Female patients of childbearing potential receiving vaccinations or boosters must have a negative urine pregnancy test on the days of vaccination, before any vaccine or booster is administered.
⁵Patients will be provided with sufficient study drug to last until their next appointment. At dose escalation visits, patients will return to the clinic to be dispensed study drug and receive new dosing instructions.
⁶Patients will be monitored for fever at every clinic visit and will monitor themselves in between visits.
⁷Patients must fast for 8 hours before blood draws at Week 24 and all Visiting Healthcare Assessments.
⁸Patients should refrain from heavy exercise 24 hours before blood collection. Walking and light exercise are acceptable.
⁹Urine pregnancy test for women of childbearing potential only.
¹⁰Site will either obtain a pre-dose or post-dose sample depending on timing of clinic visit. Actual sampling time and the most recent dose time prior to sample collection should be recorded. The sample should be collected at the same time of FD, C3, CP activity sample collection.
¹¹Refer to PRO and QoL questionnaires Healthcare Resource Utilization (HRU), EORTC-QLQ30, and WPAL: SHP will be collected only at Week 24

TABLE 8
Schedule of Assessments: Long-Term Extension, Taper, and Follow-up Periods

| | Long-term Extension | | | | | | |
|---|---|---|---|---|---|---|---|
| | VHA Visits¹ | Clinic Visits² | | | | | |
| | Weeks 28, 36, | Weeks 32, 40, | Post- | Taper⁴ | | Follow up | |
| | 44, 52, 60, 68 | 48, 56, 64, 72 | escalation³ | T1 | T2 | F/U1¹² | F/U2¹³ |
| Clinic Visit Days | | X | | X | X | X | X |
| Visiting healthcare assessment | X | | X | | | | |
| Study Drug dispensing ₅ | | X | | X | | | |
| Review safety card | X | X | X | X | X | X | X |
| Brief physical exam | | X | | | | X | X |
| Vital signs | | X | | X | X | X | X |
| (including temperature)⁶ | | | | | | | |
| Weight | | X | | | | | X |
| PRO and QoL questionnaires⁷ | | X | | | | | |
| RBC transfusion review | | X | | X | X | X | X |
| AE/SAE | X | X | X | X | X | X | X |
| Concomitant medications/protocol | X | X | X | X | X | X | X |
| restrictions | | | | | | | |
| Hematology and chemistry, and | X | X | X | X | X | X | X |
| urinalysis⁸ | | | | | | | |
| Pregnancy test⁹ | X | X | | | | | X |
| PT/PTT/INR, D-dimer | | X | | | | | X |
| Free hemoglobin, haptoglobin | | X | | | | | X |
| Direct Coombs | | X | | | | | X |
| Bb, APH10 | | X | | | | | X |
| FD, C3, CP activity¹⁰ | | X | | | | | X |
| PK sample¹¹ | | X | | | | | |
| Flow cytometry: clone size¹⁰ | | X | | | | | X |
| Flow cytometry: C3 fragment | | X | | | | | X |
| deposition¹⁰ | | | | | | | |

AE = adverse event; AP = alternative pathway; APH = AP hemolysis; CP = classical pathway; FD = factor D; F/U = follow-up; INR = international normalized ratio; PT = prothrombin time; PTT = partial thromboplastin time; QoL = quality of life; RBC = red blood cell; SAE = serious adverse event; T = taper; VHA = visiting healthcare assessment.
¹Visit window is ±7 days. Patients must fast for 8 hours before blood collection. The site will call the patient within 1 to 3 days to confirm that samples were collected and to assess AEs, SAEs, and concomitant medications.
²Visit window is ± 7 days.
³If a dose escalation occurs, blood should be drawn for measurement of alanine aminotransferase, aspartate aminotransferase, γ-glutamyl transferase, and alkaline phosphatase by the visiting healthcare service or at the clinic 72 to 96 hours after escalation.
⁴Any patient who discontinues study drug will complete taper and follow-up periods. If a patient discontinues from the study for any reason, all early termination patients should follow Week 24/ET Visit assessments.
₅ Patients will be provided with sufficient study drug to last until their next appointment. At dose escalation visits, patients will return to the clinic to be dispensed study drug and receive new dosing instructions.
⁶Patients will be monitored for fever at every clinic visit and will monitor themselves in between visits.
⁷PRO and QoL questionnaires will be administered at Week 40, 56 and 72.
⁸Patients should refrain from heavy exercise 24 hours before blood collection. Walking and light exercise are acceptable.
⁹Any positive test will be confirmed by a follow-up serum pregnancy test.
¹⁰Tests will be performed at Week 40, 56, and 72
¹¹Site will either obtain a pre-dose or post-dose sample depending on timing of clinic visit. Actual sampling time and the most recent dose time prior to sample collection should be recorded. The sample should be collected at the same time of FD, C3, CP activity sample collection.
¹²F/U1: 2 weeks post Taper Period 2 completion, physical examinations, assessment of vital signs, all required safety laboratory testing, and collection of blood and urine samples will be performed
¹³F/U2: 4 weeks after the last dose of study drug, physical examinations, assessment of vital signs, all required safety laboratory testing, and collection of blood and urine samples will be performed

Upon completion of Treatment Period 2 (Week 24), patients may enter the LTE Period for 1 year at the same danicopan dose they were receiving at Week 24, plus their C5 inhibitor therapy. During the LTE Period, patients may be dose escalated, to a maximum of 200 mg tid, at the discretion of the Principal Investigator and in consultation with the sponsor.

If a patient discontinues from the study, dosing of danicopan or placebo should be tapered over 6 days (Taper Visit 1 and 2), and two Follow-up Visits will be conducted approximately 14 days and 28 days after the last dose of study drug. Patients will continue to receive their C5 inhibitor therapy at the same dose and interval that they were receiving during the taper and follow-up visits.

Intervention Groups and Duration: Patients are randomized to danicopan or placebo, in a 2:1 ratio, for 12 weeks (Treatment Period 1) in addition to their C5 inhibitor therapy. At Week 12, patients randomized to receive placebo will be switched to danicopan for an additional 12 weeks (Treatment Period 2), and patients randomized to danicopan will continue for an additional 12 weeks. At the end of the treatment periods (24 weeks), patients may enter the LTE Period at the same dose plus their C5 inhibitor therapy. Any patient discontinuing from the study at any time point undergo a 6-day taper and follow-up for an additional 28 days.

The C5 inhibitor (eculizumab or ravulizumab) used in this study will be considered a background therapy.

Inclusion Criteria:
1. Diagnosis of PNH
2. Clinically Evident Extravascular hemolysis (EVH) defined by:
   Anemia (Hgb≤9.5 g/dL) with absolute reticulocyte count ≤120×10⁹/L.
   At least 1 packed RBC or whole blood transfusion within 6 months prior to the start of the study
3. Receiving a C5 inhibitor for at least 6 months prior to Day 1 in this study at an approved dose (or higher) and with no change in dose or interval for 224 weeks preceding Day 1 in this study. For those patients who recently switched from eculizumab to ravulizumab, they must have received at least the loading dose and 3 maintenance doses (minimum of 24 weeks) of ravulizumab preceding Day 1.
4. Platelet count ≥30,000/4 without the need for platelet transfusions.
5. Absolute neutrophil counts ≥750/μL.
6. Documentation of vaccination for Neisseria meningitidis: All patients must be vaccinated against meningococcal infections within 3 years prior to, or at the time of, initiating study drug. Patients who initiate study drug treatment less than 2 weeks after receiving a meningococcal vaccine must receive treatment with appropriate prophylactic antibiotics until 2 weeks after vaccination.
7. Age 18 years or older (or greater than or equal to minimum adult age in accordance with local legal requirements)
8. Female patients of childbearing potential must agree to use a highly effective or acceptable method of contraception from the date of signing the informed consent to 30 days after their last dose of study drug. Female patients of childbearing potential must also have a negative serum pregnancy test during Screening and negative urine pregnancy test on Day 1.
9. Female patients with documented evidence of non-childbearing potential need not employ a method of contraception.
10. Nonsterile male patients must agree to use a highly effective or acceptable method of contraception with their partner(s) of childbearing potential from the first day of dosing to 90 days after their last dose of study drug.
   Males who are surgically sterile need not employ additional contraception.
   Males must agree not to donate sperm while enrolled in this study and for 90 days after their last dose of study drug.
11. Capable of giving signed informed consent, which includes compliance with the requirements and restrictions listed in the informed consent form and in this protocol.
12. Must have access to emergency medical care.

Exclusion Criteria:
1. History of a major organ transplant (eg, heart, lung, kidney, liver) or hematopoietic stem cell transplantation (HSCT).
2. Patient's with known aplastic anemia or other bone marrow failure that requires HSCT or other therapies including anti-thymocyte globulin and/or immunosuppressants
3. Received another investigational agent other than C5 inhibitors (eculizumab or ravulizumab) within 30 days or 5 half-lives of the investigational agent prior to study entry, whichever is greater.
4. Known or suspected complement deficiency.
5. Active bacterial or viral infection, a body temperature >38° C. on two consecutive daily measures, evidence of other infection, or history of any febrile illness within 14 days prior to first study drug administration.
6. History or presence of any clinically relevant co-morbidities that would make the patient inappropriate for the study (e.g., is likely to result in deterioration of the patient's condition, affect the patient's safety during the study, or confound the results of the study).
7. Laboratory abnormalities at screening, including:

Alkaline phosphatase >2×upper limit of normal (ULN)

Alanine aminotransferase >2×ULN
Direct bilirubin >2×ULN (unless due to extravascular hemolysis, in the opinion of the Investigator and patients with Gilbert's Syndrome will be allowed into this study; however, documentation of Gilbert's Syndrome is required. If increased bilirubin is suggestive of Gilbert's syndrome, but the patient cannot provide documentation; then, the patient will be tested for this condition.
8. Any other clinically significant laboratory abnormality as judged by the Investigator that, in the opinion of the Principal Investigator, would make the patient inappropriate for the study or put the patient at undue risk.
9. Females who are pregnant, nursing, or planning to become pregnant during the study or within 90 days of study drug administration.
10. Current evidence of biliary cholestasis.
11. Evidence of human immunodeficiency virus, hepatitis B, or active hepatitis C infection at screening.
12. Estimated glomerular filtration rate <30 mL/min/1.73 m² and/or are on dialysis.
13. Hypersensitivity to the investigational drug (danicopan) or any of its excipients

Statistical Methods:

An improvement in hemoglobin levels from baseline at Week 12 for danicopan treatment is statistically compared to the improvement for placebo treatment; that is, the difference in mean changes from baseline between danicopan and placebo at Week 12.

Efficacy Analyses: Summary statistics for the baseline and post-baseline measurements, changes from baseline will be presented by visit for all continuous efficacy variables to be analyzed. Efficacy objectives and endpoints are summarized in Table 9 below.

TABLE 9
Objectives and Endpoints

| | |
|---|---|
| Objectives | Endpoints |

Primary

| | |
|---|---|
| To evaluate the efficacy of danicopan as | Change in hemoglobin (Hgb) relative to |
| compared to placebo as add-on therapy | baseline after 12 weeks of treatment |
| to a C5 inhibitor at 12 weeks | with danicopan compared to placebo |

Secondary
Key Secondary

| | |
|---|---|
| To evaluate the efficacy of danicopan as | Proportion of patients with transfusion |
| compared to placebo as add-on therapy | avoidance (TA), defined as patients who |
| to a C5 inhibitor on transfusion | remain transfusion-free and do not require |
| avoidance at 12 weeks | a transfusion as per protocol-specified |
| | guidelines through Week 12 |
| To evaluate the effect of danicopan as | Change from baseline in FACIT |
| compared to placebo as add-on therapy to | Fatigue scores at Week 12 |
| a C5 inhibitor on Functional Assessment | |
| of Chronic Illness Therapy (FACIT) | |
| Fatigue scores for 12 weeks of treatment | |
| To evaluate the effect of danicopan as | Change from baseline in absolute |
| compared to placebo as add-on therapy | reticulocyte count at Week 12 |
| to a C5 inhibitor on absolute reticulocyte | |
| count | |

Other Secondary Endpoints

| | |
|---|---|
| To evaluate the efficacy of danicopan | Change in the number of red blood cell |
| as add-on therapy to a C5 inhibitor on | (RBC) units transfused and transfusion |
| transfusion requirements at 24 weeks | instances during the 24 weeks of |
| for those patients receiving 24 weeks of | treatment with danicopan compared to the |
| danicopan | 24 weeks prior to initiation of treatment |
| | with danicopan |
| | Percentage of patients who have |
| | transfusion avoidance through 24 |
| | weeks of treatment |
| To evaluate the efficacy of danicopan as | Change in the number of RBC units |
| compared to placebo as add-on therapy | transfused and transfusion instances |
| to a C5 inhibitor on transfusion | during the 12 weeks of treatment |
| requirements at 12 weeks | with danicopan compared to the 12 |
| | weeks while receiving placebo |
| To evaluate the effect of danicopan as | Change from baseline in FACIT Fatigue |
| add-on therapy to a C5 inhibitor on | scores at Week 24 in all patients |
| Functional Assessment of Chronic | |
| Illness Therapy (FACIT) Fatigue scores | |
| for 24 weeks of treatment | |
| To assess the efficacy of danicopan | Percentage of patients with hemoglobin |
| as add-on therapy to a C5 inhibitor | stabilization during the last 12 weeks of |
| on hemoglobin stabilization | treatment in patients receiving 24 weeks |
| | of danicopan |
| To assess additional laboratory markers | Change from baseline of danicopan |
| relevant in PNH patients | treated patients compared to placebo in |
| | total and direct bilirubin at 12 weeks |
| | Changes in PNH RBC clone size, C3 |
| | fragment deposition on PNH RBCs, and |
| | measures of alternate pathway activity |
| | at 12 weeks of treatment with danicopan |
| | compared to placebo |
| | Changes in lactate dehydrogenase (LDH) |
| | and classical pathway activity at 12-weeks |
| | Percentage of patients with hemoglobin |
| | normalization at 12 weeks and 24 weeks |

Exploratory

| | |
|---|---|
| To assess Patient-Reported Outcomes | Change from baseline relative to placebo |
| (PRO) and other health-related quality | in Three-level EuroQoL 5 dimensions |
| of life (QoL) measures during 24 weeks | (EQ-5D-3L) scores at Week 12 |
| of treatment | Change from baseline in EQ-5D- |
| | 3L scores at Week 24 |
| | Change from baseline relative to placebo |
| | in European Organization for Research |
| | and Treatment of Cancer (EORTC) |
| | Quality of Life Questionnaire-Core 30 |
| | Scale (QLQ-C30) at Week 12 |
| | Change from baseline in EORTC- |
| | QLQ-C30 scale at Week 24 |
| | Change from baseline relative to placebo |
| | in Work Productivity and Activity |
| | Impairment Questionnaire: General |
| | health (WPAI: SHP, V2.0) Week 12 |
| | Change from baseline in WPAI: SHP |
| | scores at Week 24 |
| | Change from baseline relative to placebo |
| | in Healthcare Resource Utilization |
| | (HRU) at Week 12 |
| | Change from baseline in HRU scores |
| | at Week 24 |

Safety

| | |
|---|---|
| To evaluate the safety and tolerability of | Incidence of Treatment-emergent adverse |
| 24 weeks of treatment with danicopan as | events (TEAEs), serious adverse events |
| add-on therapy to a C5 inhibitor | (SAEs), laboratory abnormalities, and |
| | events leading to discontinuation of study |
| | drug during 12-week blinded and |
| | subsequent 12-week open-label treatment |
| | periods. |
| To evaluate the safety and tolerability of | Incidence of Treatment-emergent |
| with danicopan as add-on therapy to a C5 | adverse events (TEAEs), serious adverse |
| inhibitor during LTE Period | events (SAEs), laboratory abnormalities, |
| | and events leading to discontinuation of |
| | study drug |

The primary efficacy endpoint is the change in hemoglobin at Week 12 relative to baseline (defined as the lowest Hgb value, between and including screening and Day 1) between danicopan and placebo. The longitudinal changes from baseline in hemoglobin is analyzed using a mixed model for repeated measures (MMRM) which includes the fixed, categorical effects of treatment, study visit, and study visit by treatment group interaction as well as the continuous, fixed covariate of baseline hemoglobin value and the stratification randomization indicator of transfusion history in the model. The Kenward-Roger approximation will be used to estimate denominator degrees of freedom. The primary efficacy analysis will be the difference between danicopan and placebo arms at Week 12 and the test will be conducted.

Longitudinal graphic presentations will also be provided to examine the hemoglobin profile throughout 12 weeks of treatment with danicopan or placebo, plus C5 inhibitor.

The primary efficacy analysis will be based on the ITT population. A supportive analysis will be carried out for the primary efficacy endpoint, changes in hemoglobin measurement, based on the Per Protocol population to examine the impact due to major protocol deviations.

Secondary efficacy analyses will be conducted on the ITT population. Key secondary efficacy endpoints will be analyzed using a hierarchical fixed sequence test procedure to determine the statistical significance.

Safety Analyses: All safety analyses are conducted on the Safety population, both for 12-week blinded and subsequent 12-week open-label treatment periods. The safety analysis will be based primarily on the frequency of adverse events, clinical laboratory assessments, vital signs, and 12-lead ECG. Other safety data will be summarized as appropriate.

Interim Analysis: An interim analysis may be conducted at the discretion of the study sponsor (based on feasibility) when approximately 50% of patients have been randomly assigned to study treatment and have had the opportunity to complete the 12-week Treatment Period 1 (information fraction=0.5). The purpose of the interim analysis is to evaluate the study for stopping early for efficacy. If conducted, the primary endpoint of change in Hgb levels at Week 12, as well as the key secondary endpoints will be evaluated using the alpha-spending methods specified below to control family-wise error rate.

### Introduction

Danicopan (ALXN2040, previously ACH-0144471), a small molecule, orally administered, factor D (FD) inhibitor, is being developed for the treatment of complement-mediated diseases, such as paroxysmal nocturnal hemoglobinuria (PNH) and C3 glomerulopathy (C3G). Factor D is a serine protease that catalyzes the cleavage of factor B (FB), a rate-limiting step in the alternative pathway (AP) of complement. By inhibiting FD, danicopan potently and specifically inhibits AP activity. This pivotal study will assess the efficacy and safety of danicopan in patients who have clinically evident extravascular hemolysis (EVH) on a C5 inhibitor (eculizumab or ravulizumab).

Benefits

PNH is a serious, life-threatening disease, and there are unmet needs in this population that are not addressed by an approved C5 inhibitor that could potentially be addressed by an effective oral FD inhibitor. Three groups of patients whose PNH is not adequately controlled have been identified:
Patients who have a suboptimal response to eculizumab or ravulizumab (approximately 30%), presumably largely due to extravascular hemolysis that is mediated by C3 opsonization. Eculizumab or ravulizumab treatment spares the hemolytic destruction of PNH erythrocytes by the membrane attack complex (terminal stage of the complement pathway); however, it does not prevent deposition of C3 fragments on PNH erythrocyte membranes which can direct their extravascular hemolysis. Danicopan has a potential mechanistic advantage since it acts upstream of C3 cleavage and has been shown to block C3 fragment deposition.
Patients who only respond partially to eculizumab or ravulizumab due to a genetic polymorphism in CR1 (e.g., HindIII H/L and L/L genotypes), which has been postulated to result in an increased proportion of C3-opsonized RBCs, may have an improved treatment response with danicopan.
Rare patients (˜1%) with no response to eculizumab or ravulizumab due to mutations in C5 (e.g., Arg885His) could also benefit from danicopan because it acts at a different target in the complement cascade and should be unaffected by a mutation in C5.

Study Design

Overall Design

This is a multiple-region, randomized, double-blind, placebo-controlled, multiple-dose, Phase 3 study in patients with PNH who have clinically evident EVH on a C5 inhibitor (eculizumab or ravulizumab).

Screen Failures

Screen failures are defined as patients who consent to participate in the clinical study but are not subsequently entered in the study.

Study Drug

“Study Drug” in this protocol refers to danicopan or matching placebo. See Table 10.

TABLE 10
Study Interventions Administered

| | | |
|---|---|---|
| Comnound Name | Daniconan (ALXN2040) | Placebo |
| Type | Drue | Placebo |
| Dose Formulation | Tablet | Tablet |
| Unit Dose Strengths | 50 mg: 100 mg | 50 mg: 100 mg |
| Dosage Levels | 100 mg: 150 mg: 200 mg | 100 mg: 150 mg: 200 mg |
| Route of Administration | Oral | Oral |
| Sourcing | Provided bv sponser | Provided bv sponser |

Background and Concomitant Therapies

Use of specific concomitant medications other than a C5 inhibitor will be considered on a case-by-case basis with decisions made jointly between the Principal Investigator and sponsor, based on available knowledge of danicopan as well as the characteristics of the potential concomitant medication.

Background C5 Inhibitor Therapy: Eculizumab and Ravulizumab

All patients are treated with study drug in combination with a C5 inhibitor therapy (i.e., eculizumab or ravulizumab) for the purpose of data collection and analysis. C5 inhibitor used in this manner will be considered a background therapy. If patients switch to a different approved C5 inhibitor after completion of the study at Week 24, the new medication used in this manner will be also considered a background therapy, as patients are required to be on a stable dose and interval of their C5 inhibitor for a prolonged period prior to study entry.

Approved C5 inhibitor dose should not be increased, nor interval shortened, during this study (with the exception of ravulizumab weight-based dosing based on changes in weight). The dose of the C5 inhibitor may be decreased, if indicated, with a dose re-escalation to the prior dose if the dose reduction was not tolerated.

C5 inhibitor therapy will be provided according to local regulations and approvals.

In some embodiments, SOLIRIS® (eculizumab) is administered intravenously to an adult PNH patient at a dose of 600 mg weekly for four doses, followed by a dose of 900 mg at Week 5 and then at a dose of 900 mg every 2 weeks thereafter. In pediatric (<18 years of age) PNH patients, SOURIS® (eculizumab) is administered intravenously: (a) at a dose of 900 mg weekly for four doses to a subject weighing 40 kg and over, followed by a dose of 1200 mg at Week 5 and then at a dose of 1200 mg every two weeks thereafter; (b) at a dose of 600 mg weekly for two doses to a subject weighing 30 kg to less than 40 kg, followed by a dose of 900 mg at Week 3 and then at a dose of 900 mg every two weeks thereafter; (c) at a dose of 600 mg weekly for two doses to a subject weighing 20 kg to less than 30 kg, followed by a dose of 600 mg at Week 3 and then at a dose of 600 mg every two weeks thereafter; (d) at a dose of 600 mg weekly for one dose to a subject weighing 10 kg to less than 20 kg, followed by a dose of 300 mg at Week 3 and then at a dose of 300 mg every two weeks thereafter; or (e) at a dose of 300 mg weekly for one dose to a subject weighing 5 kg to less than 10 kg, followed by a dose of 300 mg at Week 2 and then at a dose of 300 mg every three weeks thereafter.

To reduce the risk of meningococcal infection, all patients are vaccinated against meningococcal infections within 3 years prior to, or at the time of, initiating study drug.

Study Drug Dose Modification

Doses of study drug may be escalated in 50-mg increments to a maximum of 200 mg tid, based on safety and clinical effect, at specified time points during the initial treatment period and during the LTE Period using the criteria below. All dose escalations using laboratory results obtained will be made at the discretion of the Principal Investigator, in consultation with the sponsor. This applies to both Treatment Periods 1 and 2. The C5 Inhibitor+Placebo Treatment Group will be escalated in the same manner as the C5 Inhibitor+Danicopan Treatment Group during the study to maintain the blind.
First dose escalation point: If the starting dose is well tolerated and the available safety data are satisfactory, a patient may be escalated to the next highest dose (to a maximum dose of 200 mg tid) if the patient's Hgb level at Week 4 or Week 16 has not increased by ?2 g/dL from their baseline value (Day 1), or the patient received a transfusion during the previous 4 weeks.
Second dose escalation point: A patient may be escalated to the next highest dose, to a maximum of 200 mg tid, if the patient's Hgb level at Week 8 or Week 20 has not increased by ?3 g/dL or has not normalized to at least the midpoint of the normal range for gender from their baseline value (Day 1), or the patient received a transfusion during the previous 4 weeks.
Third dose escalation point: A patient may be escalated to the next highest dose, to a maximum dose of 200 mg tid, if the patient's Hgb level at Week 12 or Week 24 has not normalized to at least the midpoint of the normal range for gender, or if the patient received a transfusion during the previous 4 weeks.

Blood should be drawn for measurement of ALT, AST, GGT, and ALP 72 to 96 hours after dose escalation, either in clinic or by visiting healthcare service.

Any patient who has not already been dose escalated up to 200 mg study drug, may be escalated up to a maximum of 200 mg danicopan tid, if they have been on their previous dose for at least 4 weeks and the Investigator believes that additional efficacy can be achieved. Dose escalations after Week 24 visit will be discussed with the sponsor before being implemented. If a patient has been dose escalated, the dose may be dose reduced to a lower dose for safety or tolerability reasons following consultation between the Investigator and Medical Monitor. The dose can also be re-escalated following the same process

At any time-point after the dose escalation, if patient safety or tolerability warrants dose reduction to the previous dose this may occur in consultation with the Sponsor. This dose reduction will only occur, if both Principal Investigator and the Sponsor agree that the patient will benefit from the lower dose.

Transfusion Guidelines Before and During the Study It is recommended to administer pRBC transfusion when a subject has a
1. Hemoglobin value of less than 6 g/dL regardless of presence of clinical signs or symptoms, or
2. Hemoglobin value of less than 9 g/dL with signs or symptoms of sufficient severity to warrant a transfusion.

In the event of life-threatening anemia, transfusion of ABO- and RhD-matched blood is appropriate. If further matching for Kell and JK Antigens can be conducted without delaying making the blood available for emergent transfusion, this additional testing is recommended.

Study Assessments and Procedures

The required study assessments procedures are described in this section. The timeline for all procedures may be found below.

Efficacy Assessments

Blood will be collected according to the Schedule of Assessments to assess the efficacy endpoints of change in hemoglobin, reticulocyte counts, bilirubin, and lactate dehydrogenase. PNH RBC clone size, C3 fragment deposition on PNH RBCS, AP and CP activity, and Bb, C3, and FD levels will also be assessed. Blood collection procedures are described below.

Transfusion data, including the number of RBC units transfused and the associated pretransfusion hemoglobin value (with reticulocyte count, if available) from the time of screening until follow-up will be collected (from study site records and any other location where the patient receives any transfusions) and recorded in each patient's CRF.

Patient-Reported Outcomes

All patients enrolled in the study will self-administer questionnaires for the FACIT-Fatigue (Version 4), European Organization for Research and Treatment of Cancer (EORTC) Quality of Life Questionnaire-Core 30 Scale (QLQ-C30), and EQ-5D-3L scales and Work Productivity and Activity Impairment Questionnaire: Specific Health Problem V2.0 (WPAI:SHP, V2.0) (see below). Local language versions of each of the tools will be provided separately.

Health Resource Utilization data will be collected as per schedule shown below. For HRU, the Investigator or designee will record for each participant the number of clinic visits, emergency services utilized, hospitalization, missed work and also record the number of times the patient had darkened urine.

All PROs and QoL assessments (written or electronic) will be administered before the treatment dose, during the scheduled visit.

Efficacy Analyses

Summary statistics for the baseline and post-baseline measurements, changes from baseline will be presented by pre-defined visit for all continuous efficacy variables to be analyzed.

Primary Efficacy Analysis

The primary efficacy endpoint is change in hemoglobin at Week 12 relative to baseline (defined as the lowest Hgb value, between and including screening and Day 1) between danicopan and placebo. The longitudinal changes from baseline in hemoglobin will be analyzed using a mixed model for repeated measures (MMRM) which includes the fixed, categorical effects of treatment, study visit, and study visit by treatment group interaction as well as the continuous, fixed covariate of baseline hemoglobin value and the stratification randomization indicator of transfusion history in the model. The Kenward-Roger approximation will be used to estimate denominator degrees of freedom. The primary efficacy analysis will be the contrast between Danicopan and placebo arms at Week 12 and the test will be conducted at 2-sided 0.05 significance level.

The primary objective is to evaluate the efficacy of danicopan as compared to placebo on change in hemoglobin after 12-week of treatment. To address the impact of transfusion on hemoglobin values, for patients who are transfused on or after Week 8, the hemoglobin value collected at Week 12 will not be included in the primary efficacy analysis. This rule will also apply to longitudinal observations collected at earlier visits, i.e. hemoglobin values collected within 4 weeks after transfusion will not be included in the primary efficacy analysis.

With the relatively small sample size and short duration of blinded treatment (12 weeks), all efforts will be made to minimize missing Week 12 measurements. Longitudinal graphic presentations will also be provided to examine the hemoglobin profile throughout 12 weeks of treatment with danicopan or placebo, plus an approved C5 inhibitor.

The primary efficacy analysis will be based on the ITT population. A supportive analysis will be carried out for the primary efficacy endpoint, changes in hemoglobin measurement, based on the Per Protocol population to examine the impact due to major protocol deviations. Additional sensitivity analyses will be performed to assess treatment effect under alternative missing data mechanism assumptions. The details of such analyses will be specified in the statistical analysis plan.

Secondary Efficacy Analysis

The secondary efficacy endpoints are listed below. The secondary efficacy analysis will be conducted on ITT population.

Key secondary efficacy endpoints, in order of importance, are described below. Hierarchical fixed sequence test procedure is utilized to determine the statistical significance at two-sided level of 0.05 for each endpoint sequentially.
1. Difference in proportion of patients with RBC transfusion avoidance between danicopan
   and placebo groups during the 12 weeks of treatment
2. Difference in changes from baseline in FACIT-Fatigue scores between danicopan and placebo groups at Week 12.
3. Difference in changes from baseline in absolute reticulocyte counts between danicopan and placebo groups at Week 12.

For the parameter of proportions such as patients achieving transfusion avoidance and hemoglobin normalization at Week 12, the Fisher exact test will be used to compare between danicopan and placebo arms.

For the changes in RBC transfusion units/instances from 12 weeks prior to initiation of treatment to 12-week Treatment Period 1, an analysis of covariance (ANCOVA) model including treatment arms and transfusion units/instances from 12 weeks prior to treatment initiation will be used to compare between danicopan and placebo arms.

For change from baseline in numeric endpoints such as FACIT-Fatigue scores, EQ-5D-3L scores, EORTC QLQ-C30 scores, absolute reticulocyte count, total and direct bilirubin, or other PNH-related biomarkers at Week 12 the MMRM model as specified in the primary efficacy analysis will be employed to compare the mean difference between danicopan and placebo.

The hierarchical fixed sequence test procedure calls for the current hypothesis to be rejected; that is, if the p-value for test statistic is <0.05, then proceed to test the significance of the next hypothesis from the key secondary endpoints listed above by clinical importance. The sequential testing process will be stopped when the hypothesis cannot be rejected.

Results based on various statistical procedures used to analyze data for the remaining secondary efficacy and exploratory endpoints will be descriptive as follows:
Number and proportion of patients with hemoglobin stabilization during the last 12 weeks of treatment, for patients receiving 24 weeks of danicopan treatment. Hemoglobin stabilization is defined as avoidance of no more than a 0.5 g/dL decrease in Hgb levels at Week 24 from Week 12.
Change in RBC transfusion units/instances from 24 weeks prior to initiation of treatment to 24-week treatment period for patients receiving 24 weeks of danicopan treatment.
Proportion of patients with transfusion avoidance through 24 weeks treatment period and proportion of patients with hemoglobin normalization at Week 24
Change in FACIT-Fatigue, absolute reticulocyte count, total and direct bilirubin, LDH and other PNH-related biomarkers relative to baseline (Day 1) for patients receiving 24 weeks of danicopan treatment.
PNH RBC clone size, C3 fragment deposition on PNH RBCS, measures of alternate pathway and classical pathway activity, Bb, C3, and FD levels.
PRO and QoL endpoints.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWMGEILPGSGSTEYTENFKDRVTMTRDTSTS
TVYMELSSLRSEDTAVYYCARYFFGSSPNWYFDVWGQGTLVTVSS
SEQ ID NO: 8
DIQMTQSPSSLSASVGDRVTITCGASENIYGALNWYQQKPGKAPKLLIYGATNLADGVPSRFSGSGSGTDFTLTISS
LQPEDFATYYCQNVLNTPLTFGQGTKVEIK
SEQ ID NO: 9
ASTKGPSVFPLAPCSRSTSESTAALGCLVKDYFPEPVTVSWNSGALTSGVHTFPAVLQSSGLYSLSSVVTVPSSNFG
TQTYTCNVDHKPSNTKVDKTVERKCCVECPPCPAPPVAGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLTVDKSRWQEGNVFSCSVMH
EALHNHYTQKSLSLSLGK
SEQ ID NO: 10
QVQLVQSGAEVKKPGASVKVSCKASGYIFSNYWIQWVRQAPGQGLEWMGEILPGSGSTEYTENFKDRVTMTRDTSTS
TVYMELSSLRSEDTAVYYCARYFFGSSPNWYFDVWGQGTLVTVSSASTKGPSVFPLAPCSRSTSESTAALGCLVKDY
FPEPVTVSWNSGALTSGVHTFPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEALHNHYTQKSLSLSLGK
SEQ ID NO: 11
DIQMTQSPSSLSASVGDRVTITCGASENIYGALNWYQQKPGKAPKLLIYGATNLADGVPSRFSGSGSGTDFTLTISS
LQPEDFATYYCQNVLNTPLTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 12
QVQLVQSGAEVKKPGASVKVSCKASGHIFSNYWIQWVRQAPGQGLEWMGEILPGSGHTEYTENFKDRVTMTRDTSTS
TVYMELSSLRSEDTAVYYCARYFFGSSPNWYFDVWGQGTLVTVSS
SEQ ID NO: 13
ASTKGPSVFPLAPCSRSTSESTAALGCLVKDYFPEPVTVSWNSGALTSGVHTFPAVLQSSGLYSLSSVVTVPSSNFG
TQTYTCNVDHKPSNTKVDKTVERKCCVECPPCPAPPVAGPSVFLFPPKPKDTLMISRTPEVTCVVVDVSQEDPEVQF
NWYVDGVEVHNAKTKPREEQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLTVDKSRWQEGNVFSCSVLH
EALHSHYTQKSLSLSLGK
SEQ ID NO: 14
QVQLVQSGAEVKKPGASVKVSCKASGHIFSNYWIQWVRQAPGQGLEWMGEILPGSGHTEYTENFKDRVTMTRDTSTS
TVYMELSSLRSEDTAVYYCARYFFGSSPNWYFDVWGQGTLVTVSSASTKGPSVFPLAPCSRSTSESTAALGCLVKDY
FPEPVTVSWNSGALTSGVHTFPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDIAVEWESN
GQPENNYKTTPPVLDSDGSFFLYSRLTVDKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGVHTFPAVLQSSGLYSLSSVVTVTSSNFG
TQTYTCNVDHKPSNTKVDKTVERKCCVECPPCPAPPVAGPSVFLFPPKPKDTLYITREPEVTCVVVDVSHEDPEVQF
NWYVDGMEVHNAKTKPREEQFNSTFRVVSVLTVVHQDWLNGKEYKCKVSNKGLPAPIEKTISKTKGQPREPQVYTLP
PSREEMTKNQVSLTCLVKGFYPSDIAVEWESNGQPENNYKTTPPMLDSDGSFFLYSKLTVDKSRWQQGNVFSCSVMH
EALHNHYTQKSLSLSPGK
SEQ ID NO: 16
QVQLVQSGAEVKKPGASVKVSCKASGYIFSNYWIQWVRQAPGQGLEWMGEILPGSGSTEYTENFKDRVTMTRDTSTS
TVYMELSSLRSEDTAVYYCARYFFGSSPNWYFDVWGQGTLVTVSSASTKGPSVFPLAPCSRSTSESTAALGCLVKDY
FPEPVTVSWNSGALTSGVHTFPAVLQSSGLYSLSSWTVTSSNFGTQTYTCNVDHKPSNTKVDKTVERKCCVECPPCP
APPVAGPSVFLFPPKPKDTLYITREPEVTCVVVDVSHEDPEVQFNWYVDGMEVHNAKTKPREEQFNSTFRVVSVLTV
VHQDWLNGKEYKCKVSNKGLPAPIEKTISKTKGQPREPQVYTLPPSREEMTKNQVSLTCLVKGFYPSDIAVEWESNG
QPENNYKTTPPMLDSDGSFFLYSKLTVDKSRWQQGNVFSCSVMHEALHNHYTQKSLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGEILPGSGHTEYTENFKDRVTMTRDTSTS
TVYMELSSLRSEDTAVYYCARYFFGSSPNWYFDVWGQGTLVTVSSASTKGPSVFPLAPCSRSTSESTAALGCLVKDY
FPEPVTVSWNSGALTSGVHTFPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGGIGPFFGTANYAQKFQGRVTITADESTS
TAYMELSSLRSEDTAVYYCARDTPYFDYWGQGTLVTVSS
SEQ ID NO: 28
DIELTQPPSVSVAPGQTARISCSGDSIPNYYVYWYQQKPGQAPVLVIYDDSNRPSGIPERFSGSNSGNTATLTISGT
QAEDEADYYCQSFDSSLNAEVFGGGTK LTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGIIDPDDSYTEYSPSFQGQVTISADKSIST
AYLQWSSLKASDTAMYYCARYEYGGFDIWGQGTLVTVSS
SEQ ID NO: 36
SYELTQPPSVSVAPGQTARISCSGDNIGNSYVHWYQQKPGQAPVLVIYKDNDRPSGIPERFSGSNSGNTATLTISGT
QAEDEADYYCGTYDIESYVFGGGTKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWVGAIYTGSGATYKASWAKGRFTISKDTSK
NQVVLTMTNMDPVDTATYYCASDGGYDYPTHAMHYWGQGTLVTVSS
SEQ ID NO: 44
DVVMTQSPSSLSASVGDRVTITCQASQNIGSSLAWYQQKPGQAPRLLIYGASKTHSGVPSRFSGSGSGTDFTLTISS
LQPEDVATYYCQSTKVGSSYGNHFGGGTKVEIK
SEQ ID NO: 45
QVQLVESGGGLVQPGRSLRLSCAASGFTVHSSYYMAWVRQAPGKGLEWVGAIFTGSGAEYKAEWAKGRVTISKDTSK
NQVVLTMTNMDPVDTATYYCASDAGYDYPTHAMHYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTHT
CPPCPAPELRRGPKVFLFPPKPKDTLMISRTPEVTCVVVDVSHEDPEVKFNWYVDGVEVHNAKTKPREEQYNSTYRV
VSVLTVLHQDWLNGKEYKCKVSNKGLPSSIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVLHEALHAHYTRKELSLSP
SEQ ID NO: 46
DIQMTQSPSSLSASVGDRVTITCRASQGISSSLAWYQQKPGKAPKLLIYGASETESGVPSRFSGSGSGTDFTLTISS
LQPEDFATYYCQNTKVGSSYGNTFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGEC
SEQ ID NO: 47
QVQLQESGPGLVKPSETLSLTCTVSGDSVSSSYWTWIRQPPGKGLEWIGYIYYSGSSNYNPSLKSRATISVDTSKNQ
FSLKLSSVTAADTAVYYCAREGNVDTTMIFDYWGQGTLVTVSS
SEQ ID NO: 48
AIQMTQSPSSLSASVGDRVTITCRASQGIRNDLGWYQQKPGKAPKLLIYAASSLQSGVPSRFAGRGSGTDFTLTISS
LQPEDFATYYCLQDFNYPWTFGQGTKVEIK
SEQ ID NO: 49
QVQLQESGPGLVKPSETLSLTCTVSGDSVSSSYWTWIRQPPGKGLEWIGYIYYSGSSNYNPSLKSRATISVDTSKNQ
FSLKLSSVTAADTAVYYCAREGNVDTTMIFDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESKYGPPCPPCPA
PEFLGGPSVFLFPPKPKDTLMISRTPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYAASSLQSGVPSRFAGRGSGTDFTLTISS
LQPEDFATYYCLQDFNYPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 51
DIQVTQSPSSLSASVGDRVTITCITSTDIDDDMNWYQQKPGKVPKLLISGGNTLRPGVPSRFSGSGSGTDFTLTISS
LQPEDVATYYCLQSDSLPYTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 52
EVQLVQSGPELKKPGASVKVSCKASGYTFTNYGMNWVRQAPGQGLEWMGWINTYTGETTYADDFKGRFVFSLDTSVS
TAYLQISSLKAEDTAVYYCEREGGVNNWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVEPKSCDKTHT

What is claimed is:

1. A method for treating paroxysmal nocturnal hemoglobinuria (PNH) in a subject who previously exhibited an inadequate response to C5 inhibitors, e.g., anti-C5 antibody therapy, comprising administering to the subject a therapeutically effective amount of an inhibitor of an alternate component of the alternative pathway (AP) of complement.

2. The method of claim 1, wherein the inhibitor of the alternate component of the AP comprises inhibition of a target upstream to complement 5 (C5), such as Factor D or complement 3 (C3).

3. The method of claim 1, wherein following treatment with the inhibitor of the AP, a reduction in one or more of the following is observed in the subject: (a) persistent extravascular hemolysis (EVH); (b) anemia; and/or (c) transfusion dependence; and/or an improvement in FACIT Fatigue Scale Score is observed in the subject.

4. The method of claim 3, wherein following treatment with the inhibitor of the AP, control of MAC-mediated intravascular hemolysis in the inadequately responding PNH subject is maintained or improved.

5. The method of claim 1 or claim 2, wherein the inadequate response an anti-C5 antibody therapy is related to (I) a pharmacokinetic (PK) aspect, e.g., (a) ineffective inhibition of C5 cleavage in the subject; (b) low dose and/or low subject plasma levels of the anti-C5 antibody; (c) enhanced clearance of the anti-C5 antibody in the subject; or (d) anti-C5 antibody intolerance in the subject resulting in lowered anti-C5 antibody dosing, preferably wherein anti-C5 antibody intolerance comprises fatigue and post-infusion pain; or (II) a pharmacodynamic (PD) aspect e.g., (a) CR1 polymorphism; (b) extra-vascular hemolysis (EVH), e.g., via opsonization of blood cells surviving intra-vascular hemolysis (IVH); and (c) impaired effect of anti-C5 antibody activity by C3 fragments.

6. A method for treating paroxysmal nocturnal hemoglobinuria (PNH) in a subject who previously exhibited an inadequate response to an anti-C5 antibody therapy, the method comprising:
administering to the subject a therapeutically effective amount of a complement factor D (CFD) inhibitor,
wherein the inadequate response by the subject was transfusion dependence and/or anemia; and
wherein the subject exhibits one or more of the following clinical improvements 24 weeks post-treatment with the CFD inhibitor:
(a) hemoglobin increase of 2.0 g/dL or greater compared to the subject's baseline hemoglobin level;
(b) transfusion independence; and/or
(c) Functional Assessment of Chronic Illness Therapy (FACIT) Fatigue Scale Score increase of 10 points or greater compared to the subject's baseline FACIT Fatigue Scale Score.

7. A method for treating paroxysmal nocturnal hemoglobinuria (PNH) in a subject who previously exhibited an inadequate response to an anti-C5 antibody therapy, the method comprising:
administering to the subject a therapeutically effective amount of a complement factor D (CFD) inhibitor in combination with a therapeutically effective amount of an anti-C5 antibody, or antigen binding fragment thereof,
wherein the inadequate response by the subject was transfusion dependence and/or anemia; and
wherein the subject exhibits one or more of the following clinical improvements 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater compared to the subject's baseline FACIT Fatigue Scale Score.

8. A method for treating paroxysmal nocturnal hemoglobinuria (PNH) in a subject, the method comprising:
administering to the subject a therapeutically effective amount of a complement factor D (CFD) inhibitor in combination with a therapeutically effective amount of an anti-C5 antibody, or antigen binding fragment thereof,
wherein the subject exhibits one or more of the following clinical improvements 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater compared to the subject's baseline FACIT Fatigue Scale Score.

9. The method of claim 6 or 7, wherein the subject previously exhibited an inadequate response to eculizumab.

10. The method of claim 9, wherein the subject was previously treated with eculizumab at an approved dose or higher for ≥24 weeks without change in regimen ≤8 weeks.

11. The method of claim 7 or 8, wherein the anti-C5 antibody, or antigen binding fragment thereof, administered to the subject is a human antibody, a humanized antibody, a bispecific antibody, a chimeric antibody, a Fab, a Fab′2, a ScFv, a SMIP, an AFFIBODY®, a nanobody, or a domain antibody which inhibits C5.

12. The method of claim 7 or 8, wherein the anti-C5 antibody, or antigen binding fragment thereof, administered to the subject comprises CDR1, CDR2, and CDR3 heavy chain sequences as set forth in SEQ ID NOs:1, 2, and 3, respectively, and CDR1, CDR2, and CDR3 light chain sequences as set forth in SEQ ID NOs:4, 5, and 6, respectively.

13. The method of claim 7-8 or 12, wherein the anti-C5 antibody, or antigen binding fragment thereof, administered to the subject comprises a heavy chain variable region comprising SEQ ID NO:7 and a light chain variable region comprising SEQ ID NO:8.

14. The method of claim 7-8 or 12-13, wherein the anti-C5 antibody, or antigen binding fragment thereof, administered to the subject comprises a heavy chain comprising SEQ ID NO:10 and a light chain comprising SEQ ID NO:11.

15. The method of claim 7-8 or 12-14, wherein the anti-C5 antibody administered to the subject is SOLIRIS®.

16. The method of claim 7 or 8, wherein the anti-C5 antibody, or antigen binding fragment thereof, administered to the subject comprises CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively.

17. The method of claim 7-8 or 16, wherein the anti-C5 antibody, or antigen binding fragment thereof, administered to the subject further comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

18. The method of claim 7-8 or 16-17, wherein the anti-C5 antibody, or antigen-binding fragment thereof, administered to the subject comprises a heavy chain variable region comprising SEQ ID NO:12 and a light chain variable region comprising SEQ ID NO:8.

19. The method of claim 7-8 or 16-18, wherein the anti-C5 antibody, or antigen-binding fragment thereof, administered to the subject further comprises a heavy chain constant region depicted in SEQ ID NO:13.

20. The method of claim 7-8 or 16-19, wherein the anti-C5 antibody, or antigen-binding fragment thereof, administered to the subject comprises a heavy chain polypeptide comprising the amino acid sequence depicted in SEQ ID NO:14 and a light chain polypeptide comprising the amino acid sequence depicted in SEQ ID NO:11.

21. The method of claim 7-8 or 16-20, wherein the anti-C5 antibody administered to the subject isravulizumab.

22. The method of any of the preceding claims, wherein the CFD inhibitor is a small molecule inhibitor, a nucleotide, a peptide, a protein, a peptide mimetic, an aptamer, or any other molecule that binds to Factor D.

23. The method of any of the proceeding claims, wherein the CFD inhibitor is a nucleotide selected from the group consisting of a DNA, an RNA, an shRNA, an miRNA, an siRNA, an antisense DNA.

24. The method of any one of the proceeding claims, wherein the CFD inhibitor is an antibody, or antigen-binding fragment thereof, that binds to Factor D.

25. The method of any of claims 6-22, wherein the CFD inhibitor comprises: 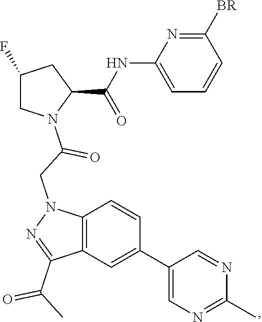
or a pharmaceutically acceptable salt thereof.

26. The method of any of claims 6-22 and 25, wherein the CFD inhibitor is danicopan.

27. The method of any of claims 6-22 and 25-26, wherein the CFD inhibitor is administered orally to the subject.

28. The method of any of claims 6-22 and 25-27, wherein the CFD inhibitor is administered orally three times daily (TID) to the subject.

29. The method of any of claims 6-22 and 25-28, wherein the CFD inhibitor is administered orally at a dose of 100 mg, 150 mg, or 200 mg TID to the subject.

30. The method of any of the preceding claims, wherein the CFD is administered for 24 weeks.

31. The method of any of the preceding claims, wherein the CDR is administered for 9 months, 12 months, 15 months, 20 months, 24 months or longer.

32. The method of any of claims 7-31 wherein the anti-C5 antibody, or antigen binding fragment, administered to the subject is administered intravenously.

33. The method of any of claims 7-15 and 22-32, wherein the anti-C5 antibody, or antigen binding fragment thereof, is administered to the subject at a dose of 600 mg weekly for four doses, followed by a dose of 900 mg at Week 5 and then at a dose of 900 mg every 2 weeks thereafter.

34. The method of any of claims 7-15 and 22-32, wherein the subject is less than 18 years of age.

35. The method of claim 34, wherein the anti-C5 antibody, or antigen binding fragment thereof, is administered to the subject less than 18 years of age:
(a) at a dose of 900 mg weekly for four doses to a subject weighing 40 kg and over, followed by a dose of 1200 mg at Week 5 and then at a dose of 1200 mg every two weeks thereafter;
(b) at a dose of 600 mg weekly for two doses to a subject weighing 30 kg to less than 40 kg, followed by a dose of 900 mg at Week 3 and then at a dose of 900 mg every two weeks thereafter;
(c) at a dose of 600 mg weekly for two doses to a subject weighing 20 kg to less than 30 kg, followed by a dose of 600 mg at Week 3 and then at a dose of 600 mg every two weeks thereafter;
(d) at a dose of 600 mg weekly for one dose to a subject weighing 10 kg to less than 20 kg, followed by a dose of 300 mg at Week 3 and then at a dose of 300 mg every two weeks thereafter; or
(e) at a dose of 300 mg weekly for one dose to a subject weighing 5 kg to less than 10 kg, followed by a dose of 300 mg at Week 2 and then at a dose of 300 mg every three weeks thereafter.

36. The method of any of claims 7-11 and 16-32 wherein the anti-C5 antibody, or antigen binding fragment thereof, is administered:
(a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and
(b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

37. The method of any of claims 7-11 and 16-32, wherein the subject is less than 18 years of age.

38. The method of claim 37, wherein the anti-C5 antibody, or antigen binding fragment thereof, is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and
(b) on Day 15 and every four weeks thereafter at a dose of 300 mg to a patient weighing
≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≤60 to <100 kg, or 3600 mg to a patient weighing ≥100 kg.

39. The method of any of claims 7-37, wherein the anti-C5 antibody, or antigen binding fragment thereof, is administered to the subject for 12 or 24 weeks.

40. The method of any of claims 7-37, wherein the anti-C5 antibody, or antigen binding fragment thereof, is administered to the subject for 9 months, 12 months, 15 months, 20 months, 24 months or longer.

41. The method of any of the preceding claims, wherein the treatment results in a shift toward normal levels of bilirubin.

42. The method of any of the preceding claims, wherein the treatment results in a reduction in reticulocytes compared to baseline.

43. The method of any of the preceding claims, wherein the treatment results in an increase in PNH specific red blood cell clone size compared to baseline.

44. The method of any of the preceding claims, wherein the treatment results in a decrease in PNH erythrocytes opsonized with C3 fragment compared to baseline.

45. The method of any one of the preceding claims, wherein the treatment results in a reduction of hemolysis as assessed by lactate dehydrogenase (LDH) levels compared to baseline.

46. The method of any of the preceding claims, wherein the treatment produces a reduction in the need for blood transfusions compared to baseline.

47. The method of any of the preceding claims, wherein the treatment results in terminal complement inhibition.

48. The method of any of the preceding claims, wherein the treatment produces at least one therapeutic effect selected from the group consisting of: a reduction or cessation in abdominal pain, dyspnea, dysphagia, chest pain and erectile dysfunction compared to baseline.

49. The method of any of the preceding claims, wherein the treatment produces a shift toward normal levels of at least one or more hemolysis-related hematologic biomarkers selected from the group consisting of: free hemoglobin, haptoglobin, reticulocyte count, PNH red blood cell (RBC) clone and/or D-dimer.

50. The method of any of the preceding claims, wherein the treatment produces a reduction in major adverse vascular events (MAVEs).

51. The method of any one of the preceding claims, wherein the treatment produces a shift toward normal levels of estimated glomerular filtration rate (eGFR) or spot urine:albumin:creatinine and plasma brain natriuretic peptide (BNP).

52. The method of any one of the preceding claims, wherein the treatment produces a change from baseline in quality of life, assessed via version 4 and the European Organisation for Research and Treatment of Cancer, Quality of Life Questionnaire-Core 30 Scale compared to baseline.

53. The method of any of the preceding claims, further comprising determining the subject's hemoglobin level, transfusion status, and/or FACIT Fatigue Scale Score at baseline and 12 or 24 weeks post-treatment, wherein
(a) a hemoglobin increase of 2.0 g/dL or greater compared to the subject's baseline hemoglobin level;
(b) transfusion independence; and/or
(c) a FACIT Fatigue Scale Score increase of 10 points or greater compared to the subject's baseline FACIT Fatigue Scale Score is indicative of treatment.

54. A method for treating paroxysmal nocturnal hemoglobinuria (PNH) in a subject who had an inadequate response to prior treatment with eculizumab, the method comprising:
administering to the subject a therapeutically effective amount of danicopan in combination with a therapeutically effective amount of eculizumab,
wherein the inadequate response by the subject was transfusion dependence and/or anemia; and
wherein danicopan is administered to the subject orally at a dose of 100 mg, 150 mg, or 200 mg TID to the subject;
wherein eculizumab is administered intravenously to the subject at a dose of 600 mg weekly for four doses, followed by a dose of 900 mg at Week 5 and then at a dose of 900 mg every 2 weeks thereafter; and
wherein the subject exhibits one or more of the following clinical improvements 12 or 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater compared to the subject's baseline FACIT Fatigue Scale Score.

55. A method for treating paroxysmal nocturnal hemoglobinuria (PNH) in a subject who had an inadequate response to prior treatment with eculizumab, the method comprising: administering to the subject a therapeutically effective amount of danicopan in combination with a therapeutically effective amount of eculizumab, wherein the inadequate response by the subject was transfusion dependence and/or anemia; and
wherein danicopan is administered to the subject orally at a dose of 100 mg, 150 mg, or 200 mg TID to the subject;
wherein eculizumab is administered intravenously to a subject less than 18 years of age:
(a) at a dose of 900 mg weekly for four doses to a subject weighing 40 kg and over, followed by a dose of 1200 mg at Week 5 and then at a dose of 1200 mg every two weeks thereafter;
(b) at a dose of 600 mg weekly for two doses to a subject weighing 30 kg to less than 40 kg, followed by a dose of 900 mg at Week 3 and then at a dose of 900 mg every two weeks thereafter;
(c) at a dose of 600 mg weekly for two doses to a subject weighing 20 kg to less than 30 kg, followed by a dose of 600 mg at Week 3 and then at a dose of 600 mg every two weeks thereafter;
(d) at a dose of 600 mg weekly for one dose to a subject weighing 10 kg to less than 20 kg, followed by a dose of 300 mg at Week 3 and then at a dose of 300 mg every two weeks thereafter; or
(e) at a dose of 300 mg weekly for one dose to a subject weighing 5 kg to less than 10 kg, followed by a dose of 300 mg at Week 2 and then at a dose of 300 mg every three weeks thereafter; and
wherein the subject exhibits one or more of the following clinical improvements 12 or 24 weeks post-treatment with the CFD inhibitor:
i. hemoglobin increase of 2.0 g/dL or greater compared to the subject's baseline hemoglobin level;
ii. transfusion independence; and/or
iii. FACIT Fatigue Scale Score increase of 10 points or greater compared to the subject's baseline FACIT Fatigue Scale Score.

56. A method for treating paroxysmal nocturnal hemoglobinuria (PNH) in a subject, the method comprising:
administering to the subject a therapeutically effective amount of danicopan in combination with a therapeutically effective amount of eculizumab,
wherein danicopan is administered to the subject orally at a dose of 100 mg, 150 mg, or 200 mg TID to the subject;
wherein eculizumab is administered intravenously to the subject at a dose of 600 mg weekly for four doses, followed by a dose of 900 mg at Week 5 and then at a dose of 900 mg every 2 weeks thereafter; and
wherein the subject exhibits one or more of the following clinical improvements 12 and/or 24 weeks post-treatment with the CFD inhibitor:
(a) hemoglobin increase of 2.0 g/dL or greater compared to the subject's baseline hemoglobin level;
(b) transfusion independence; and/or
(c) FACIT Fatigue Scale Score increase of 10 points or greater compared to the subject's baseline FACIT Fatigue Scale Score.

57. A method for treating paroxysmal nocturnal hemoglobinuria (PNH) in a subject less than 18 years of age, the method comprising administering to the subject a therapeutically effective amount of danicopan in combination with a therapeutically effective amount of eculizumab,
wherein danicopan is administered to the subject orally at a dose of 100 mg, 150 mg, or 200 mg TID to the subject;
wherein eculizumab is administered intravenously:
(a) at a dose of 900 mg weekly for four doses to a subject weighing 40 kg and over, followed by a dose of 1200 mg at Week 5 and then at a dose of 1200 mg every two weeks thereafter;
(b) at a dose of 600 mg weekly for two doses to a subject weighing 30 kg to less than 40 kg, followed by a dose of 900 mg at Week 3 and then at a dose of 900 mg every two weeks thereafter;
(c) at a dose of 600 mg weekly for two doses to a subject weighing 20 kg to less than 30 kg, followed by a dose of 600 mg at Week 3 and then at a dose of 600 mg every two weeks thereafter;
(d) at a dose of 600 mg weekly for one dose to a subject weighing 10 kg to less than 20 kg, followed by a dose of 300 mg at Week 3 and then at a dose of 300 mg every two weeks thereafter; or
(e) at a dose of 300 mg weekly for one dose to a subject weighing 5 kg to less than 10 kg, followed by a dose of 300 mg at Week 2 and then at a dose of 300 mg every three weeks thereafter; and
wherein the subject exhibits one or more of the following clinical improvements 12 and/or 24 weeks post-treatment with the CFD inhibitor:
i. hemoglobin increase of 2.0 g/dL or greater compared to the subject's baseline hemoglobin level;
ii. transfusion independence; and/or
iii. FACIT Fatigue Scale Score increase of 10 points or greater compared to the subject's baseline FACIT Fatigue Scale Score.

58. The method according any one of the preceding claims, wherein, the inhibitor of an alternate component of the alternative pathway (AP) of complement is selected from the group consisting of
a) MASP-3 inhibitor (e.g., α-MASP-3 monoclonal antibody (Mab) such as OMS906);
b) Factor D (FD) inhibitor (e.g., anti-FD Mab such as lampalizumab; or a small molecule FD inhibitor such as danicopan (ACH-4471) or BCX9930);
c) Factor B inhibitor (e.g., LNP023);
d) a compstatin molecule or a derivative thereof (e.g., APL2, APL9, AMY-101);
e) a mini Factor H (e.g., mini FH AMY-201); and
f) a factor H fusion protein (e.g., TT30).

59. The method according to any one of the preceding claims, wherein, the C5 inhibitor is selected from the group consisting of
a) an eculizumab biosimilar (e.g., ABP 959; Elizaria; SB12);
b) Nomacopan (Coversin; rVA576);
c) Ravulizumab;
d) Tesidolumab (LFG316);
e) Pozelimab; and
f) Crovalimab (SKY059).

60. The method of according to any one of the preceding claims, wherein, the alternative pathway (AP) of complement comprises a pharmaceutical composition comprising danicopan.

61. The method of according to any one of the preceding claims, comprising administering a pharmaceutical composition comprising about 100 to about 200 mg danicopan to a human subject every 8 hours.

62. The method according to any one of the preceding claims, wherein the subject exhibits extravascular hemolysis (EVH) prior to treatment.

63. A kit for treating paroxysmal nocturnal hemoglobinuria (PNH) in a subject, the kit comprising:
(a) a dose of a complement factor D (CFD) inhibitor; and
(b) instructions for using the CFD, in the method of any one of the preceding claims.

64. A kit for treating paroxysmal nocturnal hemoglobinuria (PNH) in a subject, the kit comprising:
(a) a dose of a complement factor D (CFD) inhibitor;
(b) a dose of an anti-C5 antibody; and
(c) instructions for using the CFD and anti-C5 antibody, in the method of any one of the preceding claims.

65. The kit of claim 63 or 64 wherein the CFD is danicopan.

66. The kit of claim 64 or 65, wherein the anti-C5 antibody is eculizumab.

67. The kit of claim 64 or 65, wherein the anti-C5 antibody is ravulizumab.

68. A method of treating clinically evident extravascular hemolysis (EVH) in a patient suffering from paroxysmal nocturnal hemoglobinuria (PNH), said PNH patient having previously been treated with an C5 inhibitor, e.g., anti-C5 antibody therapy, comprising administering to the subject a therapeutically effective amount of an inhibitor of an alternate component of the alternative pathway (AP) of complement.

69. The method of claim 68, wherein the inhibitor of the alternate component of the AP comprises inhibition of a target upstream to complement 5 (C5), such as Factor D or complement 3 (C3).

70. The method of claim 69, wherein the inhibitor of the Factor D inhibitor comprises danicopan.

71. The method of any one of claims 68-70, wherein the clinically evident EVH comprises (a) anemia (e.g., Hgb≤9.5 g/dL) with absolute reticulocyte count ≥120×10⁹/L; and/or (b) at least 1 packed RBC or whole blood transfusion within 6 months prior to therapy with the inhibitor of the alternate component of the AP of complement.

72. The method of any one of claims 68-71, wherein the treatment results in transfusion avoidance (TA) in the PNH patient with clinically-evident EVH.

73. The method of claim 72, wherein the treated PNH patients with clinically-evident EVH are free of pRBC transfusion requirement, e.g., a requirement that the patient undergo pRBC transfusion when the patient has a (1) hemoglobin value of less than 6 g/dL regardless of presence of clinical signs or symptoms of PNH; or (2) hemoglobin value <9 g/dL with signs or symptoms of sufficient severity to warrant a transfusion.

74. The method of any one of claims 68-73, wherein the therapeutically effective amount of the inhibitor of an alternate component of the AP complement comprises danicopan dosed at 600 mg per day.

75. The method of any one of claims 68-74, wherein the patient is treated with an anti-C5 antibody together with the therapeutically effective amount of the inhibitor of an alternate component of the AP complement.

76. The method of claim 75, wherein the anti-C5 antibody therapy comprises therapy with eculizumab (e.g., a pharmaceutical composition comprising eculizumab) or ravulizumab (e.g., a pharmaceutical composition comprising ravulizumab) per standard dosage and/or dosing schedule for each antibody in PNH therapy.
